(19) United States
(12) Patent Application Publication
(10) Pub. No.: US 2024/0024476 A1
(43) Pub. Date: Jan. 25, 2024

(54) CAR Cells and Polyspecific Binding Molecules for Treating Solid Tumor

(71) Applicants: Innovative Cellular Therapeutics Holdings, Ltd., George Town (KY); Innovative Cellular Therapeutics, Inc., Rockville, MD (US)

(72) Inventors: Chengfei Pu, Shanghai (CN); Zhiyuan Cao, Shanghai (CN); Xiaogang Shen, Shanghai (CN); Wensheng Wang, Shanghai (CN); Beibei Jia, Shanghai (CN); Dongqi Chen, Shanghai (CN); Xiaoqiang Xu, Shanghai (CN); Xudong Tang, Shanghai (CN); Wei Ding, Shanghai (CN); Xianyang Jiang, Shanghai (CN); Yuzhe Peng, Shanghai (CN); Guiting Han, Shanghai (CN); Le Tian, Rockville, MD (US); Zhao Wu, Shanghai (CN); Lei Xiao, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 18/260,376

(22) PCT Filed: Jan. 6, 2022
(86) PCT No.: PCT/US2022/070076

§ 371 Date: Jul. 5, 2023

Related U.S. Application Data
(60) Provisional application No. 63/283,074, filed on Nov. 24, 2021.
(60) Provisional application No. 63/253,785, filed on Oct. 8, 2021.
(60) Provisional application No. 63/214,538, filed on Jun. 24, 2021.
(60) Provisional application No. 63/195,369, filed on Jun. 1, 2021.
(60) Provisional application No. 63/186,411, filed on May 10, 2021.
(60) Provisional application No. 63/174,732, filed on Apr. 14, 2021.
(60) Provisional application No. 63/134,750, filed on Jan. 7, 2021.

(51) Int. Cl. A61K 39/00; C07K 14/725; C12N 5/0783; C07K 16/30; C07K 16/28; C07K 16/18; C07K 16/40; A61P 35/00

(52) U.S. Cl. CPC A61K 39/4631; C07K 14/7051; C12N 5/0636; C07K 16/3023; C07K 16/303; C07K 16/2875; C07K 16/2803; C07K 16/28; C07K 16/2851; C07K 16/18; C07K 16/40; A61P 35/00; A61K 39/4611; A61K 39/4644; A61K 39/464474; A61K 39/464417; A61K 39/464402; A61K 39/464458; A61K 39/464411; C07K 2319/03; C07K 2319/33; C07K 2317/622; A61K 2239/13; C07K 2317/31

(57) ABSTRACT

The compositions and methods described herein are directed to treating solid tumor using CAR T therapy. For example, the compositions include CAR T cells comprising an extracellular domain that binds FCR1, MSLN, GPC-3, ALPP, CD70, CLDN6, ROR1, CD205, ACPP, ADAM12, or CLDN18.2.

### CROSS REFERENCE TO RELATED APPLICATIONS

This application is claims the benefit of U.S. Provisional Application No. 63/283,074, filed Nov. 24, 2021; U.S. Provisional Application No. 63/253,785, filed Oct. 8, 2021; U.S. Provisional Application No. 63/214,538, filed Jun. 24, 2021; U.S. Provisional Application No. 63/195,369, filed Jun. 1, 2021; U.S. Provisional Application No. 63/186,411, filed May 10, 2021; U.S. Provisional Application No. 63/174,732, filed Apr. 14, 2021; and U.S. Provisional Application No. 63/134,750, filed Jan. 7, 2021; all of which are incorporated herein by reference in their entirety.

### SEQUENCE LISTING INFORMATION

A computer readable textfile, entitled “I071-0087PCT_ST25.txt,” created on or about Dec. 16, 2021 with a file size of about 1.10 MB, contains the sequence listing for this application and is hereby incorporated by reference in its entirety.

### TECHNICAL FIELD

The present disclosure relates to modified cells and uses, in particular to compositions and methods for treating cancer using Chimeric Antigen Receptor (CAR) cells.

### BACKGROUND

Most existing cancer treatment programs include surgery, radiotherapy, and chemotherapy, targeted therapy and immunotherapy. The drawbacks of the existing programs include poor treatment of advanced patients, side effects, patients with poor quality of life. For example, treatment of renal cancer includes resection, targeted therapy (anti-VEGF and mTOR inhibitor, etc.) and immunotherapy (IL-2, PD1 antibody, etc.). Treatment of pancreatic cancer includes surgical resection, radiotherapy, and chemotherapy. Treatment of urothelial carcinoma includes surgical resection, chemoradiation, targeted therapy, and immunotherapy. Treatment of breast cancer includes surgical resection, chemoradiation, targeted therapy, and immunotherapy. Treatment of ovarian cancer includes surgical resection, radiotherapy, chemotherapy, and targeted therapy. Treatment of prostate cancer includes surgical resection, chemoradiation, targeted therapy, and Immunotherapy. Treatment of esophageal cancer includes surgical resection, radiotherapy, and chemotherapy. Treatment of colorectal cancer includes surgical resection, radiotherapy, chemotherapy, and targeted therapy. Treatment of endometrial cancer includes surgical resection, radiotherapy, chemotherapy, and targeted therapy. Accordingly, there is a need to develop improved methods for treatment of cancer.

### SUMMARY

Embodiments relate to the discovery that some antigens have relatively low expression on tumor cells, as compared to their expression on normal tissues. Further, while expressed in normal tissues, these antigens are specifically expressed in a certain tissue (e.g., a group of cells or an organ), and the killing of normal cells of the tissue may not cause a life-threatening event (e.g., complications) to the subject. Examples of the nonessential tissues include organs such as prostate, breast, or melanocyte. Accordingly, the embodiments of the present disclosure relate to a chimeric antigen receptor (CAR) including an extracellular domain that binds at least one of these antigens and treating the cancer using cells including the CAR.

Embodiments relate to compositions and methods for treating cancer using CAR cells. Embodiments relate to an isolated nucleic acid sequence encoding a CAR, wherein the CAR comprises an extracellular domain, a transmembrane domain, and an intracellular domain, wherein the extracellular domain of the CAR binds an antigen of a solid tumor. The antigen can comprise FCRL1, MSLN, GPC-3, CD205, CLDN6, ALPP, ROR1, CD70, ACPP, ADAM12, or CLDN182.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows a schematic diagram illustrating an example of a CAR structure.

FIG. 2 shows an exemplary structure of a polyspecific binding molecule that interacts with target cells.

FIG. 3 shows an exemplary structure of a CAR.

FIGS. 4A, 4B, 4C, and 4D show cytokine release of ALPP CAR T cells cultured with various substrate cells.

FIGS. 5A, 5B, and 5C show cell proliferation of ALPP CAR T cells cultured with substrate cells.

FIG. 6A shows the experimental design for in vivo experiments with mice infused with ALPP CAR T cells.

FIG. 6B shows the results of ALPP expression in murine cells using immunohistochemical staining of.

FIG. 7 shows the changes in tumor volume of mice in each group over time after ALPP CAR T cell infusions.

FIGS. 8A, 8B, 8C, and 8D show cytokine release by MSLN CAR T cells cultured with substrate cells.

FIGS. 9A, 9B, and 9C show proliferation of MSLN CAR T cells cultured with substrate cells.

FIG. 10A shows the experimental design for in vivo experiments with mice infused with MSLN CAR T cells.

FIG. 10B shows the results of MSLN expression in murine cells using immunohistochemical staining.

FIG. 11 shows the changes in tumor volume of mice in each group over time after MSLN CAR T cell infusions.

FIGS. 12A, 12B, and 12C show cytokine release by CD70 CAR T cells cultured with various substrate cells.

FIGS. 13A, 13B, 13C, 13D, and 13E show proliferation of CD70 CAR T cells cultured with various substrate cells.

FIG. 14A shows the experimental design for in vivo experiments with mice infused with CD70 CAR T cells.

FIG. 14B shows results of CD70 expression in murine cells using immunohistochemical staining.

FIG. 15 shows the changes in tumor volume of mice in each group over time after CD70 CAR T cell infusions.

FIG. 16 show ACPP CAR T cells exhibited good anti-tumor activity in vitro.

FIG. 17 show ACPP CAR T cells exhibited good anti-tumor activity in vitro.

FIGS. 18A, 18B, and 18C show that ACPP CAR T cells exhibited good anti-tumor activity in mice.

FIG. 19 shows that ACPP CAR T cells exhibited good anti-tumor activity in mice.

FIGS. 20A, 20B, and 20C show the expression of FCRL1 in tissues of breast cancer (A), colorectal cancer (B), and ovarian cancer (C).

FIGS. 21A and 21B show RNA (A) and protein (B) levels of CLDN6 expression in tumor tissues.

FIGS. 22A and 22B show CLDN6 CAR T cells exhibited good anti-tumor activity in vitro.

FIG. 22A shows the construct of the vector encoding CLDN6 CAR.

FIG. 22B shows the expression of CLDN6 on the surface of HEPG2, and Huh-7, and CLDN6 was determined using CLDN6 antibodies.

FIG. 23 shows the expression of CD137, T cell activation marker, was detected 24 hours after co-culturing CAR T cells or NT cells with HepG2 or Huh7 using flow cytometry.

FIGS. 24A, 24B, and 24C show determination of IL-2, IFN-γ, and Granzyme B in the supernatant of the cultures 24 hours after co-culturing CAR T cells or NT cells with PC3-wt or PC3-CLDN6 using Cytometric Bead Array (CBA).

FIGS. 25A and 25B show that CLDN6 CAR T cells exhibited good anti-tumor effect in mice. FIG. 25A shows the experimental design of in vivo experiments with mice, and FIG. 25B shows changes in tumor volume of mice infused with Mock Group of T cells and mice infused with CLDN6 CAR T cells.

FIGS. 26A and 26B show GPC-3 expression in tumor tissues.

FIG. 27A shows the construct of the vector encoding GPC-3 CAR.

FIG. 27B shows the expression of GPC-3 on the surface of HEPG2, and Huh-7, and GPC-3, determined using GPC-3 antibodies.

FIG. 27C shows western blot results of GPC-3 expression in various cells.

FIG. 28 shows the expression of CD137, T cell activation marker, was detected 24 hours after co-culturing CAR T cells or NT cells with or without HepG2, and Huh7 using flow cytometry.

FIGS. 29A, 29B, 29C, and 29D show determination of IL-2, TNF-α, IFN-γ, and Granzyme B in the supernatant of the cultures 24 hours after co-culturing CAR T cells or NT cells with substrate cells.

FIGS. 30A, 30B, and 30C show results of CellTrace™ assay showing proliferation of CAR T cells co-cultured with substrate cells.

FIG. 31A shows the experimental design of in vivo experiments with mice.

FIG. 31B shows changes in tumor volume of mice infused with Mock Group of T cells and mice infused with GPC-3 CAR T cells.

FIGS. 32A, 32B, and 32C show that ADAM12 CAR T cells have good antitumor effect in vitro.

FIGS. 33A and 33B show that ADAM12 CAR T cells have good antitumor effect in vitro.

FIGS. 34A, 34B, and 34C show that ADAM12 CAR T cells proliferate after culturing with substrate cells.

FIGS. 35A, 35B, and 35C show that ADAM12 CAR T cells have good antitumor effect in vivo.

FIGS. 36A and 36B show that ADAM12 CAR T cells have good antitumor effect in vivo.

FIG. 37 shows the ratio of CD4+ and CD8+ cells in the peripheral blood after the mice were infused with CAR T cells.

FIGS. 38A, 38B, and 38C show CD205 CAR T exhibited good antitumor effect in vitro.

FIGS. 39A and 39B show CD205 CAR T exhibited good antitumor effect in vitro.

FIGS. 40A, 40B, and 40C show proliferation of each group of cells using CellTrace™ Cell Proliferation Kit, after CAR T cells and NT cells were co-cultured with the above positive tumor cells.

FIGS. 41A, 41B, and 41C show CD205 CAR T exhibited good antitumor effect in vivo.

FIGS. 42A and 42B show CD205 CAR T exhibited good anti-tumor effect in mice.

FIGS. 43A, and 43B show comparisons of anti-tumor activity of different infused cells.

FIG. 44 shows CAR T cells targeting CLDN18.2 showed significant anti-tumor ability in mouse DN18.2 positive tumor model.

FIGS. 45A, and 45B show in vitro proliferation of ROR1 CAR T cells. Sequences and identifiers are provided in the table below.

FIGS. 46A, 46B, 46C, and 46D show the proliferation of T cells and the reduction of tumor markers in Patient 001 after CAR T cell infusion.

FIGS. 47A, 47B, and 47C show the proliferation of T cells and CT scanning results for Patient 002 after CAR T cell infusion.

FIGS. 48A and 48B show the proliferation of T cells in Patient 003 after CAR T cell infusion.

FIG. 49 shows CT scanning results in Patient 003 after CAR T cell infusion.

FIGS. 50A, 50B, and 50C show the proliferation of T cells and the reduction of a tumor marker in Patient 004 after CAR T cells infusion.

FIGS. 51A, 51B, and 51C show the proliferation of T cells and the reduction of a tumor marker in Patient 005 after CAR T cell infusion.

FIGS. 52A and 52B show CT scanning results of Patient 005 after CAR T cells infusion.

FIGS. 53A, 53B, 53C, and 53D show the proliferation of T cells, the reduction of a solid tumor marker, and CT scanning results of Patient 006 after CAR T cells infusion.

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies; monoclonal antibodies; Fv, Fab, Fab′, and F(ab) 2 fragments; as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)₂, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be a related or unrelated to the recipient subject, but the donor subject has immune system markers which are similar to the recipient subject.

The term “xenogeneic” is used to describe a graft derived from a subject of a different species. As an example, the donor subject is from a different species than a recipient subject and the donor subject and the recipient subject can be genetically and immunologically incompatible.

The term “cancer” refers to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin (MSLN) | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma—Glioblastoma |
| EGFR | Glioma—NSCL cancer |
| tMUC 1 | Cholangiocarcinoma, Pancreatic cancer, Breast |
| | Cancer |
| PSCA | pancreas, stomach, or prostate cancer |

Throughout this specification, unless the context requires otherwise, the words “comprise,” “includes” and “including” will be understood to imply the inclusion of a stated step or element (ingredient or component) or group of steps or elements (ingredients or components) but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements, for example, the function of killing or inhibiting the growth of cancer cells. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules. The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein, and refer to any animal, such as a mammal, for example a human, or any living organism, amenable to the methods described herein. In certain non-limiting embodiments, the patient, subject, or individual is a human or mammal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. In embodiments, the disease, condition, or disorder is cancer.

The term “polynucleotide” or “nucleic acid” refers to mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Further, a Zinc finger binding domain may be fused a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcRγ were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T cell receptors” PNAS, 1993 Jan. 15; 90(2):720-4, showed that two CAR constructs in which an scFv was fused to “either the FcR gamma chain or the CD3 complex chain” triggered T cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al. (“A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl. 1, May 2014, page S57) tested a CAR molecule in which an scFv was fused to “the transmembrane and cytoplasmic domain of” a killer immunoglobulin-like receptor (KIR). Wang et al. reported that, “a KIR-based CAR targeting mesothelin (SS 1-KIR) triggers antigen-specific cytotoxic activity and cytokine production that is comparable to CD3˜-based CARs.” A second publication from the same group, Wang et al. (“Generation of Potent T-cell Immunotherapy for Cancer Using DAP12-Based, Multichain, Chimeric Immunoreceptors” Cancer Immunol Res. 2015 July; 3(7):815-26) showed that a CAR molecule in which “a single-chain variable fragment for antigen recognition was fused to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell in vitro and in vivo (in a subject). Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Embodiments of the present disclosure relate to treating cancer using chimeric antigen receptor (CAR) cells. Embodiments relate to an isolated nucleic acid encoding a CAR, wherein the CAR comprises an extracellular domain, a transmembrane domain, and an intracellular domain, wherein the extracellular domain of the CAR binds an antigen of a solid tumor. For example, transcriptional data shows that expression of antigens such as SLC6A3, KISS1R, QRFPR in normal tissues is very low, but expression of such antigens in cells related to renal cancer is high. Information of some of the antigens is provided below in

The T cell response in a subject refers to cell-mediated immunity associated with a helper, killer, regulatory, and other types T cells. For example, T cell response may include activities such as assistance to other white blood cells in immunologic processes and identifying and destroying virus-infected cells and tumor cells. T cell response in the subject can be measured via various indicators such as a number of virus-infected cells and/or tumor cells that T cells has killed, an amount of cytokines that the T cells has released in co-culturing with virus-infected cells and/or tumor cells, a level of proliferation of T cells in the subject, a phenotype change of T cells (e.g., changes to memory T cells), and a level of longevity or lifetime of T cells in the subject.

In embodiments, in vitro killing assay may be performed by measuring the killing efficacy of CAR T cells by co-culturing CAR T cells with antigen-positive cells. CAR T cells can be considered to have killing effect on the corresponding antigen-positive cells by showing a decrease in the number of corresponding antigen-positive cells co-cultured with CAR T cells and an increase in the release of cytokines such as IFNγ, TNFα, etc. as compared to control cells that do not express the corresponding antigen. Further, in vivo antitumor activity of the CAR T cells can be tested. For example, xenograft models can be established using the antigens described herein in immunodeficient mice. Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CAR T cells in vivo, immunodeficient mice bearing tumor xenografts were evaluated for CAR T cell anti-tumor activity (e.g., a decrease in mouse tumors and mouse blood IFNγ, TNFα, et al.).

The term “chimeric antigen receptor” or alternatively a “CAR” refers to a recombinant polypeptide construct comprising at least an extracellular antigen binding domain, a transmembrane domain and an intracellular signaling domain (e.g., cytoplasmic domain). In embodiments, the domains in the CAR polypeptide construct are on the same polypeptide chain (e.g., comprising a chimeric fusion protein). In embodiments, the domains of the CAR polypeptide are not on the same molecule, e.g. not contiguous with each other or are on different polypeptide chains.

In embodiments, the intracellular signaling domain may include a functional signaling domain derived from a stimulatory molecule and/or a co-stimulatory molecule as described herein. In embodiments, the intracellular signaling domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules can include cell surface molecules for inducing an efficient response from the lymphocytes (in response to an antigen).

Between the extracellular domain and the transmembrane domain of the CAR, there can be incorporated a spacer domain. As used herein, the term “spacer domain” generally means any oligo- or polypeptide that functions to link the transmembrane domain to the extracellular domain and/or the cytoplasmic domain in the polypeptide chain. A spacer domain may include up to 300 amino acids, 10 to 100 amino acids, or 25 to 50 amino acids.

The extracellular domain of a CAR may include an antigen binding domain (e.g., a scFv, a single domain antibody, or TCR, such as a TCR alpha binding domain or a TCR beta binding domain) that targets a specific tumor marker (e.g., a tumor antigen). Tumor antigens are proteins that are produced by tumor cells that elicit an immune response, particularly T cell mediated immune responses. Tumor antigens are well known in the art and include, for example, a glioma-associated antigen, carcinoembryonic antigen (CEA), 8-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the antigen that the CAR binds is CD19, the CAR thereof is referred to as CD19CAR.

In embodiments, the extracellular ligand-binding domain comprises a scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID NO: 338), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides comprising about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor. In embodiments, the binding element of the CAR can include any antigen binding moiety that when bound to its cognate antigen, affects a tumor cell such that the tumor cell fails to grow, or is forced to die or diminish.

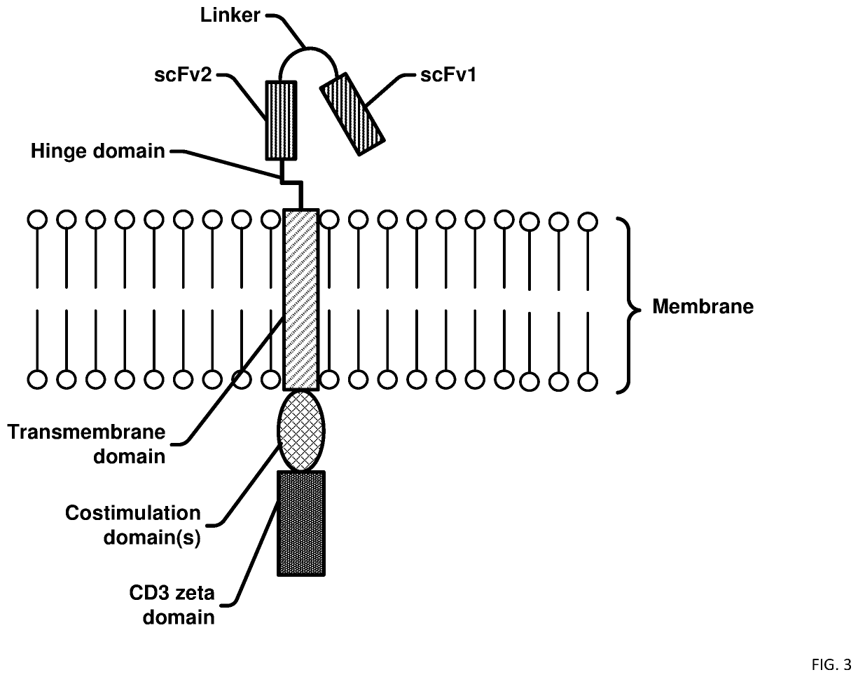

The present disclosure also relates to a bispecific chimeric antigen receptor (See FIG. 3), a polynucleotide encoding the bispecific chimeric antigen receptor, and/or a modified cell comprising the polynucleotide, wherein the bispecific chimeric antigen receptor comprises a first antigen binding domain, a second antigen binding domain, a cytoplasmic domain, and a transmembrane domain, and wherein the first antigen binding domain recognizes a first antigen, and the second antigen binding domain recognize a second antigen. In embodiments, the first antigen is an antigen associated with a white blood cell, and the second antigen is a solid tumor antigen. In embodiments, the first and second antigens are identical or different. In embodiments, the first and second antigens are both solid tumor antigens.

In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises a CD3 zeta signaling domain. Embodiments relate to a vector comprising the isolated nucleic acid sequence described herein. Embodiments relate to an isolated cell comprising the isolated nucleic acid sequence described herein.

Embodiments relate to a composition comprising a population of cells including T cells comprising the CAR described herein. Embodiments relate to a CAR encoded by the isolated nucleic acid sequence described herein. In embodiments, an isolated nucleic acid sequence encodes a chimeric antigen receptor (CAR), wherein the CAR comprises an extracellular domain, a transmembrane domain, and an intracellular domain, wherein the extracellular domain binds an antigen of a tumor (e.g., solid tumor). In embodiments, the extracellular domain binds FCRL1, MSLN, GPC-3, CD205, ALPP, CLCN6, and CD70. In embodiments, the CAR comprises one of the amino acid sequences of Tables 2-4. In embodiments, a method of eliciting and/or enhancing T-cell response in a subject having a solid tumor or treating a solid tumor in the subject, the method comprising administering an effective amount of T cells comprising the CAR.

Mesothelin (MSLN) is a glycosylphosphatidylinositol-anchored cell-surface protein that can function as a cell adhesion protein. This protein is overexpressed in epithelial mesotheliomas, ovarian cancers and in specific squamous cell carcinomas.

ALPP is a metalloenzyme that catalyzes the hydrolysis of phosphoric acid monoesters. The protein is primarily expressed in placental and endometrial tissue; however, strong ectopic expression has been detected in ovarian adenocarcinoma, serous cystadenocarcinoma, and other ovarian cancer cells.

GPC-3 (Glypican-3, or GPC3), a member of the glypican-related integral membrane proteoglycan family (GRIPS), contains a core protein anchored to the cytoplasmic membrane via a glycosyl phosphatidylinositol linkage. GPC-3 plays a role in the control of cell division and growth regulation. The protein encoded by the GPC-3 gene can bind to and inhibit the dipeptidyl peptidase activity of CD26, and it can induce apoptosis in certain cell types. In embodiments, various CARs were designed to target GPC-3. In embodiments, a CAR comprises at least one of SEQ ID NOs: 16-21 and 148-177, and the CAR binds SEQ ID NO: 15.

CD70 is a cytokine belonging to the tumor necrosis factor (TNF) ligand family. This cytokine is a ligand for TNFRSF27/CD27. It is a surface antigen on activated T and B lymphocytes, but not on resting, T and B lymphocytes. It induces proliferation of co-stimulated T cells, enhances the generation of cytolytic T cells, and contributes to T cell activation. This cytokine is also reported to play a role in regulating B cell activation, cytotoxic function of natural killer cells, and immunoglobulin synthesis. Examples of CD70 CAR include 1) a traditional carrier (41-BB and CD3 zeta) followed by a CD70 scFv, 2) a traditional carrier connected to the extracellular segment of CD27, and 3) CD27 FL C-terminal integrated with a CD3 zeta (e.g., CD27 and CD3 zeta). In embodiments, various CARs were designed to target CD70. In embodiments, a CAR comprises at least one of SEQ ID NOs: 2, 3, 85-108, and 271-297, and the CAR binds SEQ ID NO: 1.

CD205 is a type I endocytic receptor protein to direct captured antigens from the extracellular space to a specialized antigen-processing compartment. CD205 has been found to be expressed in breast cancer such as HER2 negative breast cancer, triple negative breast cancer, pancreatic cancer, and bladder cancer. In embodiments, various CARs were designed to target CD205. In embodiments, a CAR comprises at least one of SEQ ID NOs: 5-10 and 109-114, and the CAR binds SEQ ID NO: 4.

ADAM12 (A Disintegrin and Metalloprotease 12) is upregulated in human breast cancers and is a predictor of chemoresistance in estrogen receptor-negative tumors. ADAM12 is induced during epithelial-to-mesenchymal transition, a feature associated with claudin-low breast tumors, which are enriched in cancer stem cell (CSC) markers. In embodiments, various CARs were designed to target ADAM12. In embodiments, a CAR comprises at least one of SEQ ID NOs: 38-40 and 52-57, and the CAR binds SEQ ID NO: 37.

ALPP (Alkaline phosphatase, placental type), mainly expressed in placenta and testis, is overexpressed on certain tumor cell types, such as on a proportion of colorectal cancers. In embodiments, various CARs were designed to target ALPP. In embodiments, a CAR comprises at least one of SEQ ID NOs: 42-44 and 68-84, and the CAR binds SEQ ID NO: 41.

Prostatic Acid Phosphatase (PAP), an enzyme expressed by the Acid Phosphatase, Prostate (ACPP) gene, is predominantly produced in the prostate, and is an important biomarker that is used to assess and monitor prostate cancer. In embodiments, various CARs were designed to target PAP. In embodiments, a CAR comprises at least one of SEQ ID NOs: 49-51.

CLDN18.2 (claudin-18 isoform 2) is a protein that in humans is encoded by the CLDN18 gene and belongs to the family of claudin proteins. The claudin family of proteins are transmembrane proteins of the tight junction that control pracellular diffusion within the epithelium. In embodiments, various CARs were designed to target CLDN18.2. In embodiments, a CAR comprises at least one of SEQ ID NOs: 46-48 and 298-318, and the CAR binds SEQ ID NO: 45.

CLDN6 (Claudin 6) is a Protein Coding gene. Diseases associated with CLDN6 include Hepatitis C Virus and Monosomy 22. Gene Ontology (GO) annotations related to this gene include identical protein binding and structural molecule activity. In embodiments, various CARs were designed to target CLDN6. In embodiments, a CAR comprises at least one of SEQ ID NOs: 12-14 and 115-117, and the CAR binds SEQ ID NO: 11.

MSLN (Mesothelin) is a Protein Coding gene. Diseases associated with MSLN include Asbestosis and Malignant Pleural Mesothelioma. In embodiments, various CARs were designed to target MSLN. In embodiments, a CAR comprises at least one of SEQ ID NOs: 23-28 and 198-230, and the CAR binds SEQ ID NO: 22.

MUC16 (Mucin 16, Cell Surface Associated Protein) is a Protein Coding gene. Diseases associated with MUC16 include Clear Cell Adenocarcinoma. In embodiments, various CARs were designed to target MUC16. In embodiments, a CAR comprises at least one of SEQ ID NOs: 30-32 and 231-242, and the CAR binds SEQ ID NO: 29.

ROR1 (Receptor Tyrosine Kinase Like Orphan Receptor 1) is a Protein Coding gene. Diseases associated with ROR1 include deafness. In embodiments, various CARs were designed to target ROR1. In embodiments, a CAR comprises at least one of SEQ ID NOs: 34-36 and 256-270, and the CAR binds SEQ ID NO: 33.

AFP (Alpha-fetoprotein) is used as a tumor marker to help detect and diagnose cancers of the liver, testicles, and ovaries. In embodiments, various CARs were designed to target AFP. In embodiments, a CAR comprises at least one of SEQ ID NOs: 59-67, and the CAR binds SEQ ID NO: 58.

DLL3 (Delta-like ligand 3) is an inhibitory Notch ligand that is highly expressed in Small Cell Lung Cancer (SCLC) and other neuroendocrine tumors. In embodiments, various CARs were designed to target DLL3. In embodiments, a CAR comprises at least one of SEQ ID NOs: 119-136, and the CAR binds SEQ ID NO: 118.

FAP (Fibroblast activation protein), also known as prolyl endopeptidase, is an enzyme that in humans is encoded by the FAP gene. FAP may acts as a tumor promoter via epithelial-mesenchymal transition (EMT) in human oral squamous cell carcinoma. In embodiments, various CARs were designed to target FAP. In embodiments, a CAR comprises at least one of SEQ ID NOs: 138-143 and 319-336, and the CAR binds SEQ ID NO: 137.

HAVCR1 (Hepatitis A Virus Cellular Receptor 1) is a Protein Coding gene. Diseases associated with HAVCR1 include Hepatitis A. In embodiments, various CARs were designed to target HAVCR1. In embodiments, a CAR comprises at least one of SEQ ID NOs: 179-187, and the CAR binds SEQ ID NO: 178.

LRRC15 (Leucine-rich repeat-containing protein 15) is also known as LIB and hLIB. LRRC15 is highly expressed in a variety of solid tumors. In embodiments, various CARs were designed to target LRRC15. In embodiments, a CAR comprises at least one of SEQ ID NOs: 189-197, and the CAR binds SEQ ID NO: 188.

MUC17 (Mucin 17, Cell Surface Associated) is a Protein Coding gene. Diseases associated with MUC17 include Biliary Papillomatosis. In embodiments, various CARs were designed to target MUC17. In embodiments, a CAR comprises at least one of SEQ ID NOs: 244-255, and the CAR binds SEQ ID NO: 243.

GPC-2 (Glypican-2, GPC2) is a Protein Coding gene. Diseases associated with GPC-2 include Central Corneal Ulcer and Omodysplasia. In embodiments, various CARs were designed to target GPC2. In embodiments, a CAR comprises at least one of SEQ ID NOs: 145-147, and the CAR binds SEQ ID NO: 144.

The cells, including CAR cells and modified cells, described herein can be derived from a stem cell. The stem cells may be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T cell selected from the group consisting of inflammatory T lymphocytes, cytotoxic T lymphocytes, regulatory T lymphocytes, and helper T lymphocytes. In embodiments, the cells can be derived from the group consisting of CD4+T-lymphocytes and CD8+T-lymphocytes. Prior to proliferation and genetic modification of the cells described herein, a source of cells can be obtained from a subject through a variety of non-limiting methods. T cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, the cells can be derived from a healthy donor, from a patient diagnosed with cancer or from a patient diagnosed with an infection. In embodiments, the cells are part of a mixed population of cells which present different phenotypic characteristics.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells may be distinguished on the basis of their origin and/or on the extent of their capacity for differentiation into other types of cell. Stem cells can include embryonic stem (ES) cells (i.e., pluripotent stem cells), somatic stem cells, induced pluripotent stem cells, and any other types stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). Somatic stem cells have been regarded as having a capacity for differentiation lower than that of the pluripotent ES cells. The capacity of fetal stem cells for differentiation is greater than that of adult stem cells. Somatic stem cells apparently differentiate into only a limited number of different types of cells and have been described as multipotent. Tissue-specific stem cells normally give rise to only one type of cell. In contrast, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (iPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin cells, and blood cells.

In embodiments, the CAR cells, the modified cell, or the cell is a T cell, a NK cell, a macrophage or a dendritic cell. For example, the CAR cells, the modified cell, or the cell is a T cell.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCR chains. In embodiments, a T cell clone that expresses a TCR with high affinity for the target antigen is isolated. In embodiments, tumor-infiltrating lymphocytes (TILs) or peripheral blood mononuclear cells (PBMCs) is cultured in the presence of antigen-presenting cells (APCs) pulsed with a peptide representing an epitope known to elicit a dominant T cell response when presented in the context of a defined HLA allele. High-affinity clones can then be selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) can then be generated and tested for functionality (antigen specificity and functional avidity) and used to produce a clinical lot of the vector. An aliquot of the final product is then used to transduce the target T cell population (generally purified from patient PBMCs), which is expanded before infusion into the subject.

In embodiments, the binding element of the CAR can include any antigen binding moiety that when bound to its cognate antigen, affects a tumor cell for example, it kills the tumor cell, inhibits the growth of the tumor cell, or promotes death of the tumor cell.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The embodiments of the present disclosure further relate to vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for delivering nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence.

Additional information related to expression of synthetic nucleic acids encoding CARs and gene transfer into mammalian cells is provided in U.S. Pat. No. 8,906,682, incorporated by reference in its entirety.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 10⁴ to 10⁹ cells/kg body weight, preferably 10⁹ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In embodiments, it may be desired to administer activated T cells to a subject and then subsequently redraw the blood (or have apheresis performed), collect the activated and expanded T cells, and reinfuse the patient with these activated and expanded T cells. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, certain populations of T cells may be selected.

The administration of the pharmaceutical compositions described herein may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i. v.) injection, or intraperitoneally. In embodiments, the T cell compositions of the present disclosure are administered to a patient by intradermal or subcutaneous injection. In embodiments, the T cell compositions of the present disclosure are preferably administered by i.v. injection. The compositions of T cells may be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In embodiments, the cell compositions of the present disclosure are administered following B-cell ablative therapy such as agents that react with CD20, e.g., Rituxan. For example, subjects may undergo standard treatment with high dose chemotherapy followed by peripheral blood stem cell transplantation. In embodiments, following the transplant, subjects receive an infusion of the expanded immune cells of the present disclosure. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

In embodiments, the population of cells described herein is used in autologous CAR T cell therapy. In embodiments, the CAR T cell therapy is allogenic CAR T cell therapy, TCR T cell therapy, and NK cell therapy.

Embodiments relate to an in vitro method for preparing modified cells. The method includes obtaining a sample of cells from the subject. For example, the sample can include T cells or T cell progenitors. The method can further include transfecting the cells with a DNA encoding at least a CAR, culturing the population of CAR cells ex vivo in a medium that selectively enhances proliferation of CAR-expressing T cells.

The sample can be a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

Embodiments of the present disclosure relate to treating cancer using Chimeric Antigen Receptor (CAR) cells using a molecule associated with a gene fusion. Embodiments relate to an isolated nucleic acid sequence encoding a chimeric antigen receptor (CAR), wherein the CAR comprises an extracellular domain, a transmembrane domain, and an intracellular domain, wherein the extracellular domain binds a gene fusion neoantigen.

As used herein, the term “gene fusion” refers to the fusion of at least a portion of a gene to at least a portion of an additional gene. The gene fusion need not include entire genes or exons of genes. In some instances, gene fusion is associated with alterations in cancer. A gene fusion product refers to a chimeric genomic DNA, a chimeric messenger RNA, a truncated protein or a chimeric protein resulting from a gene fusion. The gene fusion product can be detected by various methods described in U.S. Pat. No. 9,938,582, which is incorporated herein by reference. A “gene fusion neoantigen” refers to a truncated protein or a chimeric protein that results from a gene fusion. In embodiments, an epitope of a gene fusion neoantigen can include a part of the gene fusion neoantigen or an immunogenic part of another antigen caused by the gene fusion. In embodiments, the gene fusion neoantigen interacts with, or is a part of the cell membrane. In embodiments, the gene fusion neoantigen is associated with tumor or cancer.

In embodiments, detection of mRNA and protein expression levels of the target molecules in human cells may be performed using experimental methods such as qPCR and FACS. Further, target molecules specifically expressed in the corresponding tumor cells with very low expression or undetectable expression in normal tissue cells may be identified.

In embodiments, In Vitro Killer Assay as well as killing experiment of CAR T cells co-cultured with antigen-positive cells can be performed. CAR T cells can exhibit a killing effect on the corresponding antigen-positive cells, a decrease in the number of corresponding antigen-positive cells co-cultured with CAR T cells, and an increase in the release of IFNγ, TNFα, etc. as compared to control cells that do not express the corresponding antigen.

In embodiments, In Vivo Killer Assay can be performed. For example, mice can be transplanted with corresponding tumor cells expressing an antigen, and transfused with CAR T cells that targets the antigen on the tumor cells. A decrease in mouse tumors and mouse blood IFNγ, TNFα, and other signals can be defected after the transfusion of the CAR T cells.

Embodiments relate to a method of eliciting and/or enhancing T cell response in a subject having a solid tumor or treating a solid tumor in the subject, the method comprising administering an effective amount of T cells comprising the CAR described herein. In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain.

Embodiments relate to a vector comprising the isolated nucleic acids described herein.

Embodiments relate to an isolated cell comprising the isolated nucleic acid sequence described herein. Embodiments relate to a composition comprising a population of cells comprising the CAR described herein. The population of cells can be a population of T cells. Embodiments relate to a CAR encoded by the isolated nucleic acid sequence described herein. Embodiments relate to a method of eliciting and/or enhancing T cell response in a subject or treating a tumor of the subject, the method comprising: administering an effective amount of T cell comprising the CAR described herein.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody has twelve CDRs altogether for binding antigens. In embodiments, the CAR molecules comprise one or more CDRs of FCRL1, MSLN, GPC-3, CD205, ALPP, and CD70.

The present disclosure describes modified cells that include one or more different antigen binding domains. The modified cells can include at least two different antigen binding domains: a first antigen binding domain for expanding and/or maintaining the genetically modified cells, and a second antigen binding domain for killing a target cell, such as a tumor cell. For example, the first antigen binding domain binds a surface marker, such as a cell surface molecule of a white blood cell (WBC) (e.g., CD19), and the second antigen binding domain binds a target antigen on tumor cells. In embodiments, the cell surface molecule is a surface antigen of a WBC. In embodiments, the target antigen on tumor cells comprise one or more of FCRL1, MSLN, GPC-3, CD205, ALPP, and CD70. The at least two antigen binding domains may be located on the same or different modified cells. For example, the modified cells can include a modified cell including a CAR binding CD19, a modified cell including a CAR binding to ACPP, a modified cell including a CAR binding CD19 and ACPP, and/or a modified cell including two CARs that separately binds CD19 and ACPP. In embodiments, the modified cells may be used to treat a subject having cancer.

FCRL1 (Fc receptor-like protein 1), also known as CD307a, contains two immunoreceptor tyrosine activation motifs and can act as an activated co-receptor in B cells. FCRL1 is mainly expressed in normal lymphoid tissues, especially B cells, while it is not expressed in other normal tissues. FCRL1 is highly expressed in many cancer tissues, such as colorectal cancer, breast cancer, and ovarian cancer. FCRL1 is selectively expressed on the surface of CD19+ mature B cells. Through RT-PCR, it was found that FCRL1 began to be expressed in pre-B cells, the expression level was higher in naïve and memory B cells, and the expression level was low in the pre-germinal center (pre-GC), GC B cells, and plasma cell populations. FCRL1 is highly expressed in both B cells and cancer tissues. In embodiments, FCRL1CAR is designed to recognize the antigens of B cells and cancer cells at the same time and perhaps replace CoupledCAR® to achieve the same effect. Thus, in embodiments, FCRL1 can be used as the target for CoupledCAR® system described in PCT Publication NOs: WO2020146743 and WO2020106843, which are incorporated herein by reference in their entirety. FCRL1CAR T can inhibit the growth of tumor cells expressing FCRL1 and stimulate the proliferation of CAR T cells due to its binding B cells.

In embodiments, the modified cells described herein includes a CAR molecule comprising at least two different antigen binding domains. The CAR molecule can be a bispecific CAR molecule. For example, the two antigen binding domains can be on the same CAR molecule, on different CAR molecules, or on a CAR molecule and T cell receptor (TCR). A single CAR can include at least two different antigen binding domains, or the two different antigen binding domains are each on a separate CAR molecule. The at least two different antigen binding domains can be on the same CAR molecule or different CAR molecules, but in the same modified cell. Moreover, the at least two different antigen binding domains can be on a CAR molecule and a T cell receptor in the same modified cell. In embodiments, the bispecific CAR molecule can include a binding domain binding an antigen of WBC (e.g., CD19) and a binding domain binding a solid tumor antigen. In embodiments, the bispecific CAR molecule can include two binding domains binding two different solid tumor antigens.

In embodiments, the at least two different antigen binding domains are on different CAR molecules which are expressed by different modified cells. Further, the one or more different antigen binding domains are on a CAR molecule and a T cell receptor, which are expressed by different modified cells.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### Exemplary Embodiments

The following are exemplary embodiments.

1. An isolated nucleic acid encoding a chimeric antigen receptor (CAR), wherein the CAR comprises an extracellular domain, a transmembrane domain, and an intracellular domain, wherein the extracellular domain binds an antigen of a tumor (e.g., solid tumor).

2. The isolated nucleic acid of embodiment 1, wherein the extracellular domain binds FCRL1.

3. The isolated nucleic acid of embodiment 1 or 2, wherein the extracellular domain binds human FCRL1.

4. The isolated nucleic acid of any one of embodiments 1-3, wherein the CAR binds amino acid sequence SEQ ID NO: 337.

5. A method of eliciting and/or enhancing T-cell response in a subject having the solid tumor or treating the solid tumor of the subject, the method comprising administering an effective amount of T cells comprising the isolated nucleic acid of any one of embodiments 2-4.

6. The isolated nucleic acid or the method of any one of embodiments 1-5, wherein the tumor is associated with colorectal cancer, breast cancer, ovarian cancer.

7. The isolated nucleic acid of embodiment 1, wherein the extracellular domain binds MSLN.

8. The isolated nucleic acid of embodiment 1 or 7, wherein the extracellular domain binds human MSLN.

9. The isolated nucleic acid of any one of embodiments 1, 7, or 8 wherein the CAR comprises one of the amino acid sequences SEQ ID NOs: 23-28 and 198-230.

10. A method of eliciting and/or enhancing T-cell response in a subject having a solid tumor or treating a solid tumor in the subject, the method comprising administering an effective amount of T cells comprising the isolated nucleic acid of any one of embodiments 1 or 7-9.

11. The isolated nucleic acid or the method of any one of embodiments 1 or 7-10, wherein the tumor is associated with pancreatic cancer or lung cancer.

12. The isolated nucleic acid of embodiment 1, wherein the extracellular domain binds GPC-3.

13. The isolated nucleic acid of embodiment 1 or 12, wherein the extracellular domain binds human GPC-3.

14. The isolated nucleic acid of any one of embodiments 1, 12, or 13, wherein the CAR comprises one of the amino acid sequences SEQ ID NOs: 16-21 and 148-177.

15. A method of eliciting and/or enhancing T-cell response in a subject having the solid tumor or treating the solid tumor of the subject, the method comprising administering an effective amount of T cells comprising the isolated nucleic acid of any one of embodiments 1 or 12-14.

16. The isolated nucleic acid or the method of any one of embodiments 1 or 12-15, wherein the tumor is associated with liver cancer.

17. The isolated nucleic acid of embodiment 1, wherein the extracellular domain binds ALPP.

18. The isolated nucleic acid of embodiment 1 or 17, wherein the extracellular domain binds human ALPP.

19. The isolated nucleic acid of any one of embodiments 1, 17, or 18, wherein the CAR comprises one of the amino acid sequences SEQ ID NOs: 42-44 and 68-84.

20. A method of eliciting and/or enhancing T cell response in a subject having a solid tumor or treating a solid tumor in the subject, the method comprising administering an effective amount of T cells comprising the isolated nucleic acid of any one of embodiments 17-19.

21. The isolated nucleic acid or the method of any one of suitable embodiments 1 and 17-20, wherein the tumor is associated with endometrial cancer and/or ovarian cancer.

22. The isolated nucleic acid of embodiment 1, wherein the extracellular domain binds CD70, ROR1, ACPP, ADAM12, or CLDN18.2.

23. The isolated nucleic acid of embodiment 1 or 22, wherein the extracellular domain binds human CD70, human ROR1, human ACPP, human ADAM12, or CLDN18.2.

24. The isolated nucleic acid of any one of embodiments 1, 22, or 23, wherein
the extracellular domain binds CD70 and the CAR comprises one of the amino acid sequences SEQ ID NOs: 2, 3, 85-108, and 271-297;
the extracellular domain bind ROR1 and the CAR comprises one of the amino acid sequences SEQ ID NOs: 35, 36, 257, 258, 260, 261, 263, 264, 266, 267, 269, and 270.
the extracellular domain binds ACPP, and the CAR comprises one of the amino acid sequences SEQ ID NOs: 50 and 51;
the extracellular domain binds ADAM12, and the CAR comprises one of the amino acid sequences SEQ ID NO: 53, 54, 57, and 57; or the extracellular domain binds CLDN18.2, and the CAR comprises one of the amino acid sequences 47 and 48.

25. A method of eliciting and/or enhancing T cell response in a subject having the solid tumor or treating the solid tumor of the subject, the method comprising administering an effective amount of T cells comprising the isolated nucleic acid of any one of embodiments 1 or 22-24.

26. The isolated nucleic acid or the method of any one of embodiments 1 and 22-25, wherein the tumor is associated with kidney cancer.

27. The isolated nucleic acid of embodiment 1, wherein the extracellular domain binds CLDN6.

28. The isolated nucleic acid of embodiment 1 or 27, wherein the extracellular domain binds human CLDN6.

29. The isolated nucleic acid of any one of embodiments 1, 27, or 28, wherein the CAR comprises one of the amino acid sequences SEQ ID NOs: 12-14, and 115-117.

30. A method of eliciting and/or enhancing T cell response in a subject having the solid tumor or treating the solid tumor of the subject, the method comprising administering an effective amount of T cells comprising the isolated nucleic acid of any one of embodiments 1 or 27-29.

31. The isolated nucleic acid or the method of any one of embodiments 1 and 27-30, wherein the tumor is associated with liver cancer.

32. The isolated nucleic acid of embodiment 1, wherein the extracellular domain binds CD205.

33. The isolated nucleic acid of embodiment 1 or 32, wherein the extracellular domain binds human CD205.

34. The isolated nucleic acid of any one of embodiments 1, 32, or 33, wherein the CAR comprises one of the amino acid sequences SEQ ID NOs: 8-10, 110, 111, 113, and 114.

35. A method of eliciting and/or enhancing T cell response in a subject having the solid tumor or treating the solid tumor of the subject, the method comprising administering an effective amount of T cells comprising the isolated nucleic acid of any one of embodiments 1 or 32-34.

36. The isolated nucleic acid or the method of any one of embodiments 1 and 32-35, wherein the tumor is associated with breast cancer, pancreatic cancer, and bladder cancer.

37. A method of implementing CoupledCAR® system described in PCT Publication NOs: WO2020146743 and WO2020106843, wherein the CAR comprises at least one scFv listed in Tables 2-4.

38. An isolated nucleic acid encoding a chimeric antigen receptor (CAR), wherein the CAR comprises an extracellular domain, a transmembrane domain, and an intracellular domain, wherein the extracellular domain binds a gene fusion neoantigen, and optionally wherein the gene fusion comprises a fusion of at least a portion of a first gene to at least a portion of a second gene and wherein optionally the gene fusion neoantigen is associated with a tumor.

39. The isolated nucleic acid or the method of any one of embodiments 1-38, wherein the intracellular domain comprises a co-stimulatory signaling region comprising an intracellular signaling domain of CD27, CD28, 4-1BB, OX40, CD30, CD40, PD-1, ICOS, lymphocyte function-associated antigen-1 (LFA-1), CD2, CD7, LIGHT, NKG2C, B7-H3, or any combination thereof.

40. The isolated nucleic acid or the method of any one of embodiments 1-39, wherein the intracellular domain comprises a CD3 zeta signaling domain.

41. A vector comprising the isolated nucleic acid of any one of embodiments 1-40.

42. A CAR encoded by the isolated nucleic acid or vector of any one embodiments 1-41.

43. A modified cell comprising the isolated nucleic acid, the vector, or the CAR of any one of embodiments 1-42, optionally the modified cell comprises a modified T cell.

44. A population of modified cells comprising the modified cell of embodiment 43, optionally wherein the population of modified cells comprises modified T cells.

45. A composition comprising the isolated nucleic acid, the CAR, the vector, the modified, or the population of modified cells of any one of embodiments 1-44, optionally the composition comprises a population of modified T cells.

46. A method of eliciting and/or enhancing T cell response in a subject having a solid tumor or treating a solid tumor of a subject, the method comprising administering an effective amount of T cells comprising the isolated nucleic acid, vector, the CAR of any one of embodiments 1-42, an effective amount of the modified T cells or the population of modified T cells of embodiment 43 or 44, or an effective amount of the composition of embodiment 45.

47. The modified cell, the population of modified cells, the composition comprising the modified cells or the population of modified cells, or the method of any one of embodiments 43-45, wherein the modified cell comprises or is a T cell derived from a healthy donor or a subject having cancer.

48. The modified cell, the population of modified cells, the composition comprising the modified cells or the population of modified cells, or the method of any one of embodiments 43-47, wherein the modified cell further comprises a dominant negative form of a receptor associated with an immune checkpoint inhibitor, and optionally the modified cell is a modified T cell.

49. The modified cell, the population of modified cells, the composition comprising the modified cells or the population of modified cells, or the method of embodiments 48, wherein the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRD, natural killer cell receptor 2B4 (2B4), and CD 160.

50. The isolated nucleic acid, modified cell, optionally modified T cell, or the method of embodiment 48 or 49, wherein immune checkpoint inhibitor is modified PD-1.

51. The isolated nucleic acid, modified cell, optionally modified T cell, or the method of embodiment 49 or 50, wherein the modified PD-1 lacks a functional PD-1 intracellular domain for PD-1 signal transduction, interferes with a pathway between PD-1 of a human T cell of the human cells and PD-L1 of a certain cell, comprises or is a PD-1 extracellular domain or a PD-1 transmembrane domain, or a combination thereof, or a modified PD-1 intracellular domain comprising a substitution or deletion as compared to a wild-type PD-1 intracellular domain, or comprises or is a soluble receptor comprising a PD-1 extracellular domain that binds PD-L1 of a certain cell.

52. The isolated nucleic acid, modified cell, optionally modified T cell, or the method of any one of embodiments 49-51, wherein an inhibitory effect of PD-L1 on cytokine production of the human T cells of the population is less than an inhibitory effect of PD-L1 on cytokine production of human T cells that do not comprise at least a part of the nucleic acid sequence that encodes the modified PD-1.

53. The isolated nucleic acid, modified cell, optionally modified T cell, or the method of one of embodiments 1-52, wherein the modified cell is a modified T cell that is engineered to express and secrete a therapeutic agent, and optionally wherein the therapeutic agent is a cytokine or a small protein.

54. The modified T cell or the method of embodiment 53, wherein the therapeutic agent is or comprises IFN-γ.

55. The modified T cell or the method of embodiment 53 or 54, wherein the therapeutic agent is or comprises at least one of IL-6, IFN-γ, IL-17, and CCL19.

56 The modified T cell or the method of any one of embodiments 53-55, wherein the therapeutic agent is or comprises IL-15 or IL-12, or a combination thereof.

57. The modified T cell or the method of any one of embodiments 53-56, wherein the therapeutic agent is or comprises a recombinant or native cytokine.

58. The modified T cell or the method of embodiment 53, wherein the therapeutic agent comprises a FC fusion protein associated with a small protein.

59. The modified T cell or the method of embodiment 53 or 58, wherein the cytokine is or comprises IL-12, IL-15, IL-6 or IFN-γ.

60. The modified T cell or the method of any one of embodiments 53-59, wherein the therapeutic agent is regulated by Hif1a, NFAT, FOXP3, and/or NFkB.

61. The modified T cell or the method of any one of embodiments 53-60, wherein the therapeutic agent is or comprises two or more recombinant or native cytokines are connected via 2A or IRES component.

62. The isolated nucleic acid, modified cell, or the method of any one of embodiments 1-61, wherein the modified cell, optionally modified T cell, comprises a first targeting vector and a second targeting vector, the first targeting vector comprising a nucleic acid encoding a CAR binding a blood antigen and a therapeutic agent, and the second targeting vector comprises a nucleic acid encoding a CAR binding a solid tumor antigen and a dominant negative form of an immune checkpoint molecule.

63. The isolated nucleic acid, modified cell, or the method of one of embodiments 1-62, wherein the modified cell, optionally modified T cell, comprises a first targeting vector and a second targeting vector, the first targeting vector comprising a nucleic acid encoding a CAR binding CD19 and the therapeutic agent, and the second targeting vector comprises a nucleic acid encoding a CAR biding UPK2, ACPP, SIGLEC15 or KISS1R and a dominant negative form of PD-1.

64. The isolated nucleic acid, modified cell, or the method of one of embodiments 1-63, wherein the modified cell, optionally modified T cell, comprises a first targeting vector and a second targeting vector, the first targeting vector comprising a nucleic acid encoding a CAR binding a blood antigen, and the second targeting vector comprises a nucleic acid encoding a CAR biding solid tumor antigen.

65. The isolated nucleic acid, modified cell, or the method of one of embodiments 1-63, wherein the modified cell, optionally modified T cell, comprises a first targeting vector and a second targeting vector, the first targeting vector comprising a nucleic acid encoding a CAR binding a B cell antigen, and the second targeting vector comprises a nucleic acid encoding a CAR biding solid tumor antigen.

66. The isolated nucleic acid, modified cell, or the method of one of embodiments 1-65, wherein the modified cell, optionally modified T cell, comprises a nucleic acid encoding hTERT, SV40LT, or a combination thereof.

67. The isolated nucleic acid, modified T cell, or the method of embodiment 66, wherein the modified T cell is more proliferable than T cells without nucleic acid sequence.

68. The isolated nucleic acid, modified T cell, or the method of embodiment 67, wherein the modified T cell remains functions of normal T cells/CAR T cells such as cell therapy functions.

69. The isolated nucleic acid, modified T cell or the method of any one of embodiments 66-68, wherein the T cell comprises a CAR and is cultured in the presence of an agent that is recognized by the extracellular domain of the CAR.

70. The isolated nucleic acid, modified cell, optionally modified T cell, or the method of any one of embodiments 1-69, wherein modified cell, optionally modified T cell, comprises genomic integration of the nucleic acid sequence encoding hTERT, a nucleic acid encoding SV40LT, or a combination thereof, and optionally wherein the cell comprises constitutive expression of hTERT, SV40LT, or a combination thereof.

71. The isolated nucleic acid, modified T cell, optionally modified T cell, or the method of any one of embodiments 1-70, wherein expression of hTERT, SV40LT, or a combination thereof, is regulated by an inducible expression system such as a rtTA-TRE system.

72. The isolated nucleic acid, modified T cell or the method of any one of embodiments 1-71, wherein modified T cell comprises a nucleic acid sequence encoding a suicide gene such as a an HSV-TK system.

73. The isolated nucleic acid, modified cell, optionally modified T cell, or the method of any one of embodiments 1-72, wherein the modified cell, optionally modified T cell, has a reduced graft-versus-host disease (GVHD) response in a bioincompatible human recipient as compared to the GVHD response of the primary human T cell.

74. The isolated nucleic acid, modified cell, optionally modified T cell, or the method of any one of embodiments 1-72, wherein the modified cell, optionally modified T cell, has a reduced expression of endogenous TRAC gene.

75. The isolated nucleic acid, modified cell, optionally modified T cell, or the method of any one of embodiments 1-72, wherein the CAR comprises a scFv or CAR sequence listed in Table 2.

76. A population of cells comprising the modified cells of any one of suitable embodiments 1-75.

77. A kit comprising the nucleic acid, the vector, the CAR, the modified cell, the population of modified cells, the composition, and/or the pharmaceutical composition of any one of the embodiments 1-76.

78. Use of the nucleic acid, the vector, the CAR, the modified cell, the population of modified cells, the composition, the pharmaceutical composition, the kit, or the method of any one of embodiments 1-77 for use in a method of treating a subject's body by therapy.

79. The use of embodiment 78, wherein the subject is a mammal, and optionally wherein the mammal is a human.

80. The use of embodiment 78 or 79, wherein the subject is suffering from or diagnosed with cancer.

81. The use of any one of embodiments 78-80, wherein the use comprises eliciting and/or enhancing a T cell response in the subject.

82. Use of the nucleic acid, the vector, the CAR molecule, the modified cells, the population of modified cells, the compositions, the pharmaceutical compositions, the kit, or the methods of any one of embodiments 1-81 for use in a method of eliciting and/or enhancing a T cell response in a subject.

83. The use of embodiment 82, wherein the subject is a mammal, and optionally wherein the mammal is a human.

84. The use of embodiment 82 or 83, wherein the subject is suffering from or diagnosed with cancer.

85. A polyspecific binding molecule (PBM), wherein the PBM comprises at least a first binding domain binding a T cell and at least a second binding domain comprises a scFv described in Table 2-4.

86. A method of treating cancer, the method comprising administering an effective amount of PBM (e.g., BiTE®) of embodiment 85 to a subject having a form of cancer, more information of PBM can be found at ICT's PCT Application NO: PCT/US21/28429, which is incorporated herein by its entity.

87. An isolated nucleic acid encoding a polyspecific binding molecule comprising a scFv or a CAR comprising an extracellular domain comprising the scFv, a transmembrane domain, and an intracellular domain, the extracellular domain binds an antigen.

88. A CAR comprising an extracellular domain comprising a scFv, a transmembrane domain, and an intracellular domain, the extracellular domain binds an antigen.

89. A cell comprising the isolated nucleic acid of embodiment 1 or a CAR of embodiment 87.

90. A pharmaceutical composition comprising a population of the cells of embodiment 88.

91. A method of cause T cell response in a subject in need thereof and/or treating a tumor of the subject, the method comprising administering an effective amount of the pharmaceutical composition of embodiment 90 to the subject.

92. The isolated nucleic acid, CAR, cell, pharmaceutical composition, and the method of any of embodiments 87-91, wherein:
the antigen is GPC-3 (Glypican-3), a CAR or scFv comprises at least one of SEQ ID NOs: 16-21 and 148-177, and/or the CAR binds SEQ ID NO: 15;
the antigen is CD70, a CAR or scFv comprises at least one of SEQ ID NOs: 2, 3, 85-108, and 271-297, and/or the CAR binds SEQ ID NO: 1;
the antigen is CD205, a CAR or scFv comprises at least one of SEQ ID NOs: 5-10 and 109-114, and/or the CAR binds SEQ ID NO: 4;
the antigen is ADAM12, a CAR or scFv comprises at least one of SEQ ID NOs: 52-57, and/or the CAR binds SEQ ID NO: 37;
the antigen is ALPP, a CAR or scFv comprises at least one of SEQ ID NOs: 42-44 and 68-84, and/or the CAR binds SEQ ID NO: 41;
the antigen is Prostatic Acid Phosphatase (PAP), a CAR or scFv comprises at least one of SEQ ID NOs: 49-51;
the antigen is CLDN18.2, a CAR or scFv comprises at least one of SEQ ID NOs: 46-48 and 298-318, and/or the CAR binds SEQ ID NO: 45;
the antigen is CLDN6, a CAR or scFv comprises at least one of SEQ ID NOs: 12-14 and 115-117, and/or the CAR binds SEQ ID NO: 11;
the antigen is MSLN, a CAR or scFv comprises at least one of SEQ ID NOs: 23-28 and 198-230, and/or the CAR binds SEQ ID NO: 22;
the antigen is MUC16, a CAR or scFv comprises at least one of SEQ ID NOs: 30-32 and 231-242, and/or the CAR binds SEQ ID NO: 29;
the antigen is ROR1, a CAR or scFv comprises at least one of SEQ ID NOs: 34-36 and 256-270, and/or the CAR binds SEQ ID NO: 33;
the antigen is ACPP, a CAR or scFv comprises at least one of SEQ ID NO: 49-51;
the antigen is AFP, a CAR or scFv comprises at least one of SEQ ID NOs: 59-67, and/or the CAR binds SEQ ID NO: 58;
the antigen is DLL3, a CAR or scFv comprises at least one of SEQ ID NOs: 119-136, and/or the CAR binds SEQ ID NO: 118;
the antigen is FAP, a CAR or scFv comprises at least one of SEQ ID NOs: 138-143 and 319-336, and/or the CAR binds SEQ ID NO: 137;
the antigen is HAVCR1, a CAR or scFv comprises at least one of SEQ ID NOs: 179-184, and/or the CAR binds SEQ ID NO: 178; or the antigen is LRRC15, a CAR or scFv comprises at least one of SEQ ID NOs: 189-197, and/or the CAR binds SEQ ID NO: 188.

93. The isolated nucleic acid, cell, pharmaceutical composition, and the method of any of embodiments 87-92, wherein the PBM further comprise another binding domain binding CD3, more information about the PBM is provided in PCT Patent Application PCT/US2021/028429, which is incorporated by its entirety.

### EXAMPLES

### Example 1. Construction of CAR T Cells and Assay Thereof

Lentiviral vectors that encode individual CAR molecules were generated and transfected with T cells, and the expression of CARs was confirmed by flow cytometry assay. Techniques related to cell cultures, construction of cytotoxic T lymphocyte assay may be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in its entirety.

Primary T cells were transduced with lentivirus including various CARs to establish different CAR T cell lines targeting different antigens listed in Tables 2-4. These cells were obtained from healthy human donors. The lentivirus included nucleic acid sequences encoding CAR molecules, and further included the IRES-mCherry (green) construct, which encodes green fluorescence for confirmation of CAR expression. Techniques related to cell cultures, construction of lentiviral vectors, and flow cytometry may be found in “Treatment of Advanced Leukemia in Mice with mRNA-Engineered T Cells, HUMAN GENE THERAPY 22:1575-1586 (December 2011)”, which is incorporated herein by reference.

Each type of CAR T cells and the corresponding type of antigen-expressed substrate cells were co-cultured, and CAR T cells' responses induced by the substrate cells were measured. A ratio of E:T 1:1/3:1/10:1/30:1 (i.e., CAR T cells: target tumor cells) of CAR T cells and target tumor cells were co-cultured for 24 hours. The supernatant was collected, and the release of IFN-γ was measured. Among the tested CAR molecules and their corresponding substrate cells, IFN-γ release were observed in multiple combinations of CAR molecules and substrate cells expressing antigens. The sequences of these CARs and the corresponding antigens are listed in Tables 2 and 3. However, IFN-γ release is not obvious when CAR T cells expressing CAR molecules and wild-type substrate cells were co-cultured. Techniques related to cell cultures, construction of cytotoxic T-lymphocyte assay may be found in “Control of large, established tumor xenografts with genetically retargeted human T cells containing CD28 and CD137 domains,” 3360-3365 PNAS Mar. 3, 2009, vol. 106 no. 9, which is incorporated herein by reference.

CAR T cell killing assays were conducted to measure the effectiveness of CAR T cells expressing the CAR molecules listed in Tables 2 and 3. As compared with non-transduced T cells, these CAR T cells significantly reduced the numbers of antigen-expression cells.

Subcutaneous tumor models were constructed on mice by subcutaneously injecting tumor cells to mice. These mice inoculated with tumor cells were randomly divided into Mock group infused with no T cells, NT group infused with non-transduced T cells, and CAR T group infused with T cells expressing the CAR molecules listed in Table 2. These three groups of mice were given an equal volume of distilled phosphate buffered saline (DPBS), NT cells, and CAR T cells through the tail vein, respectively. The body weight of the mice was recorded regularly, the size of the tumor was measured, and blood was collected from the marginal venous plexus every week for flow cytometry to detect the proliferation of T cells in the peripheral blood. Techniques related to cell cultures, construction of cytotoxic T lymphocyte assay may be found in “Control of large, established tumor xenografts with genetically retargeted human T cells containing CD28 and CD137 domains,” PNAS, Mar. 3, 2009, vol. 106 no. 9, 3360-3365, which is incorporated herein by reference in its entirety.

Table 2 lists various sequence identifiers and their sequences for establishing various anti-antigen CAR T cells.

| Identifiers | SEQ ID NO |
|---|---|
| CD70 antigen | 1 |
| CD70 binding domain | 2 |
| CD70 CAR | 3 |
| CD205 antigen | 4 |
| CD205 binding domain 2 | 8 |
| CD205 CAR 3 | 9 |
| CD205 CAR 4 | 10 |
| CD205 CAR 9 | 339 |
| CLDN6 antigen | 11 |
| CLDN6 binding domain | 12 |
| CLDN6 binding domain 2 | 13 |
| CLDN6 CAR 1 | 14 |
| GPC3 antigen | 15 |
| GPC3 binding domain 1 | 16 |
| GPC3 CAR 1 | 17 |
| GPC3 CAR 2 | 18 |
| GPC3 binding domain 2 | 19 |
| GPC3 CAR 3 | 20 |
| GPC3 CAR 4 | 21 |
| MSLN antigen | 22 |
| MSLN binding domain 1 | 23 |
| MSLN CAR 1 | 24 |
| MSLN CAR 2 | 25 |
| MSLN binding domain 2 | 26 |
| MSLN CAR 3 | 27 |
| MSLN CAR 4 | 28 |
| MUC16 antigen | 29 |
| MUC16 binding domain | 30 |
| MUC16 CAR 1 | 31 |
| MUC16 CAR 2 | 32 |
| ROR1 antigen | 33 |
| ROR1 binding domain | 34 |
| ROR1 CAR 1 | 35 |
| ROR1 CAR 2 | 36 |
| ADAM12 antigen | 37 |
| ALPP antigen | 41 |
| ALPP binding domain | 42 |
| ALPP CAR 1 | 43 |
| ALPP CAR 1 | 44 |
| CLDN18.2 antigen | 45 |
| CLDN18.2 binding domain | 46 |
| CLDN18.2 CAR 1 | 47 |
| CLDN18.2 CAR 2 | 48 |
| ACPP binding domain | 49 |
| ACPP CAR 1 | 50 |
| ACPP CAR 2 | 51 |

Table 3 lists various sequence identifiers and their sequences for establishing various anti-antigen CAR T cells.

| Identifiers | SE ID NO: |
|---|---|
| ADAM12 binding domain 2 | 52 |
| ADAM12 CAR 3 | 53 |
| ADAM12 CAR 4 | 54 |
| ADAM12 binding domain 3 | 55 |
| ADAM12 CAR 5 | 56 |
| ADAM12 CAR 6 | 57 |
| AFP antigen | 58 |
| AFP binding domain | 59 |
| AFP CAR 1 | 60 |
| AFP CAR 2 | 61 |
| AFP binding domain 2 | 62 |
| AFP CAR 3 | 63 |
| AFP CAR 4 | 64 |
| AFP binding domain 3 | 65 |
| AFP CAR 5 | 66 |
| AFP CAR 6 | 67 |
| ALPP binding domain 2 | 68 |
| ALPP CAR 3 | 69 |
| ALPP CAR 4 | 70 |
| ALPP binding domain 3 | 71 |
| ALPP CAR 5 | 72 |
| ALPP CAR 6 | 73 |
| ALPP binding domain 4 | 74 |
| ALPP CAR 7 | 75 |
| ALPP CAR 8 | 76 |
| ALPP binding domain 5 | 77 |
| ALPP CAR 9 | 78 |
| ALPP CAR 10 | 79 |
| ALPP binding domain 6 | 80 |
| ALPP CAR 11 | 81 |
| ALPP CAR 12 | 82 |
| ALPP CAR 13 | 83 |
| ALPP CAR 14 | 84 |
| CD70 binding domain 2 | 85 |
| CD70 CAR 3 | 86 |
| CD70 CAR 4 | 87 |
| CD70 binding domain 3 | 88 |
| CD70 CAR 5 | 89 |
| CD70 CAR 6 | 90 |
| CD70 binding domain 4 | 91 |
| CD70 CAR 7 | 92 |
| CD70 CAR 8 | 93 |
| CD70 binding domain 5 | 94 |
| CD70 CAR 9 | 95 |
| CD70 CAR 10 | 96 |
| CD70 binding domain 6 | 97 |
| CD70 CAR 11 | 98 |
| CD70 CAR 12 | 99 |
| CD70 binding domain 7 | 100 |
| CD70 CAR 13 | 101 |
| CD70 CAR 14 | 102 |
| CD70 binding domain 8 | 103 |
| CD70 CAR 15 | 104 |
| CD70 CAR 16 | 105 |
| CD70 binding domain 9 | 106 |
| CD70 CAR 17 | 107 |
| CD70 CAR 18 | 108 |
| CD205 antigen 3 | 109 |
| CD205 CAR 5 | 110 |
| CD205 CAR 6 | 111 |
| CD205 antigen 3 | 112 |
| CD205 CAR 5 | 113 |
| CD205 CAR 6 | 114 |
| CLDN6 CAR 2 | 115 |
| CLDN6 CAR 3 | 116 |
| CLDN6 CAR 4 | 117 |
| DLL3 antigen | 118 |
| DLL3 binding domain | 119 |
| DLL3 CAR 1 | 120 |
| DLL3 CAR 2 | 121 |
| DLL3 binding domain 2 | 122 |
| DLL3 CAR 3 | 123 |
| DLL3 CAR 4 | 124 |
| DLL3 binding domain 3 | 125 |
| DLL3 CAR 5 | 126 |
| DLL3 CAR 6 | 127 |
| DLL3 binding domain 4 | 128 |
| DLL3 CAR 7 | 129 |
| DLL3 CAR 8 | 130 |
| DLL3 binding domain 5 | 131 |
| DLL3 CAR 9 | 132 |
| DLL3 CAR 10 | 133 |
| DLL3 binding domain 6 | 134 |
| DLL3 CAR 11 | 135 |
| DLL3 CAR 12 | 136 |
| FAP Antigen | 137 |
| FAP binding domain 1 | 138 |
| FAP CAR 1 | 139 |
| FAP CAR 2 | 140 |
| FAP binding domain 2 | 141 |
| FAP CAR 3 | 142 |
| FAP CAR 4 | 143 |
| GPC2 antigen | 144 |
| GPC2 binding domain | 145 |
| GPC2 CAR 1 | 146 |
| GPC2 CAR 2 | 147 |
| GPC3 binding domain 2 | 148 |
| GPC3 CAR 3 | 149 |
| GPC3 CAR 4 | 150 |
| GPC3 binding domain 3 | 151 |
| GPC3 CAR 5 | 152 |
| GPC3 CAR 6 | 153 |
| GPC3 binding domain 4 | 154 |
| GPC3 CAR 7 | 155 |
| GPC3 CAR 8 | 156 |
| GPC3 binding domain 5 | 157 |
| GPC3 CAR 9 | 158 |
| GPC3 CAR 10 | 159 |
| GPC3 binding domain 6 | 160 |
| GPC3 CAR 11 | 161 |
| GPC3 CAR 12 | 162 |
| GPC3 binding domain 7 | 163 |
| GPC3 CAR 13 | 164 |
| GPC3 CAR 14 | 165 |
| GPC3 binding domain 8 | 166 |
| GPC3 CAR 15 | 167 |
| GPC3 CAR 16 | 168 |
| GPC3 binding domain 9 | 169 |
| GPC3 CAR 17 | 170 |
| GPC3 CAR 18 | 171 |
| GPC3 binding domain 10 | 172 |
| GPC3 CAR 19 | 173 |
| GPC3 CAR 20 | 174 |
| GPC3 binding domain 11 | 175 |
| GPC3 CAR 21 | 176 |
| GPC3 CAR 22 | 177 |
| HAVCR1 antigen | 178 |
| HAVCR1 binding domain | 179 |
| HAVCR1 CAR 1 | 180 |
| HAVCR1 CAR 2 | 181 |
| HAVCR1 binding domain 2 | 182 |
| HAVCR1 CAR 3 | 183 |
| HAVCR1 CAR 4 | 184 |
| LRRC15 antigen | 188 |
| LRRC15 binding domain 1 | 189 |
| LRRC15 CAR 1 | 190 |
| LRRC15 CAR 2 | 191 |
| LRRC15 binding domain 2 | 192 |
| LRRC15 CAR 3 | 193 |
| LRRC15 CAR 4 | 194 |
| LRRC15 binding domain 3 | 195 |
| LRRC15 CAR 5 | 196 |
| LRRC15 CAR 6 | 197 |
| MSLN binding domain 3 | 198 |
| MSLN CAR 5 | 199 |
| MSLN CAR 6 | 200 |
| MSLN binding domain 4 | 201 |
| MSLN CAR 7 | 202 |
| MSLN CAR 8 | 203 |
| MSLN binding domain 5 | 204 |
| MSLN CAR 9 | 205 |
| MSLN CAR 10 | 206 |
| MSLN binding domain 6 | 207 |
| MSLN CAR 11 | 208 |
| MSLN CAR 12 | 209 |
| MSLN binding domain 7 | 210 |
| MSLN CAR 13 | 211 |
| MSLN CAR 14 | 212 |
| MSLN binding domain 8 | 213 |
| MSLN CAR 15 | 214 |
| MSLN CAR 16 | 215 |
| MSLN binding domain 9 | 216 |
| MSLN CAR 17 | 217 |
| MSLN CAR 18 | 218 |
| MSLN binding domain 10 | 219 |
| MSLN CAR 19 | 220 |
| MSLN CAR 20 | 221 |
| MSLN binding domain11 | 222 |
| MSLN CAR 21 | 223 |
| MSLN CAR 22 | 224 |
| MSLN binding domain 12 | 225 |
| MSLN CAR 23 | 226 |
| MSLN CAR 24 | 227 |
| MSLN binding domain 13 | 228 |
| MSLN CAR 25 | 229 |
| MSLN CAR 26 | 230 |
| MUC16 Binding domain 2 | 231 |
| MUC16 CAR 3 | 232 |
| MUC16 CAR 4 | 233 |
| MUC16 Binding domain 3 | 234 |
| MUC16 CAR 5 | 235 |
| MUC16 CAR 6 | 236 |
| MUC16 Binding domain 4 | 237 |
| MUC16 CAR 7 | 238 |
| MUC16 CAR 8 | 239 |
| MUC16 Binding domain 5 | 240 |
| MUC16 CAR 9 | 241 |
| MUC16 CAR 10 | 242 |
| MUC17 Antigen | 243 |
| MUC17 Binding domain 1 | 244 |
| MUC17 CAR 1 | 245 |
| MUC17 CAR 2 | 246 |
| MUC17 Binding domain 2 | 247 |
| MUC17 CAR 3 | 248 |
| MUC17 CAR 4 | 249 |
| MUC17 Binding domain 3 | 250 |
| MUC17 CAR 5 | 251 |
| MUC17 CAR 6 | 252 |
| MUC17 Binding domain 4 | 253 |
| MUC17 CAR 7 | 254 |
| MUC17 CAR 8 | 255 |
| ROR1 binding domain 2 | 256 |
| ROR1 CAR 3 | 257 |
| ROR1 CAR 4 | 258 |
| ROR1 binding domain3 | 259 |
| ROR1 CAR 5 | 260 |
| ROR1 CAR 6 | 261 |
| ROR1 binding domain 4 | 262 |
| ROR1 CAR 7 | 263 |
| ROR1 CAR 8 | 264 |
| ROR1 binding domain5 | 265 |
| ROR1 CAR9 | 266 |
| ROR1 CAR 10 | 267 |
| ROR1 binding domain6 | 268 |
| ROR1 CAR 11 | 269 |
| ROR1 CAR 12 | 270 |

Table 4 lists various sequence identifiers and their sequences for establishing various anti-antigen CAR T cells.

| | |
|---|---|
| CD70 binding domain 10 | 271 |
| CD70 CAR 19 | 272 |
| CD70 CAR 20 | 273 |
| CD70 binding domain 11 | 274 |
| CD70 CAR 21 | 275 |
| CD70 CAR 22 | 276 |
| CD70 binding domain 12 | 277 |
| CD70 CAR 23 | 278 |
| CD70 CAR 24 | 279 |
| CD70 binding domain 13 | 280 |
| CD70 CAR 25 | 281 |
| CD70 CAR 26 | 282 |
| CD70 binding domain 14 | 283 |
| CD70 CAR 27 | 284 |
| CD70 CAR 28 | 285 |
| CD70 binding domain 15 | 286 |
| CD70 CAR 29 | 287 |
| CD70 CAR 30 | 288 |
| CD70 binding domain 16 | 289 |
| CD70 CAR 31 | 290 |
| CD70 CAR 32 | 291 |
| CD70 binding domain 17 | 292 |
| CD70 CAR 33 | 293 |
| CD70 CAR 34 | 294 |
| CD70 binding domain 18 | 295 |
| CD70 CAR 35 | 296 |
| CD70 CAR 36 | 297 |
| CLDN18.2 binding domain 2 | 298 |
| CLDN18.2 CAR 3 | 299 |
| CLDN18.2 CAR 4 | 300 |
| CLDN18.2 binding domain 3 | 301 |
| CLDN18.2 CAR 5 | 302 |
| CLDN18.2 CAR 6 | 303 |
| CLDN18.2 binding domain 4 | 304 |
| CLDN18.2 CAR 7 | 305 |
| CLDN18.2 CAR 8 | 306 |
| CLDN18.2 binding domain 5 | 307 |
| CLDN18.2 CAR 9 | 308 |
| CLDN18.2 CAR 10 | 309 |
| CLDN18.2 binding domain6 | 310 |
| CLDN18.2 CAR 11 | 311 |
| CLDN18.2 CAR 12 | 312 |
| CLDN18.2 binding domain7 | 313 |
| CLDN18.2 CAR 13 | 314 |
| CLDN18.2 CAR 14 | 315 |
| CLDN18.2 binding domain8 | 316 |
| CLDN18.2 CAR 15 | 317 |
| CLDN18.2 CAR 16 | 318 |
| FAP binding domain 3 | 319 |
| FAP CAR 5 | 320 |
| FAP CAR 6 | 321 |
| FAP binding domain 4 | 322 |
| FAP CAR 5 | 323 |
| FAP CAR 6 | 324 |
| FAP binding domain 5 | 325 |
| FAP CAR 5 | 326 |
| FAP CAR 6 | 327 |
| FAP binding domain 6 | 328 |
| FAP CAR 5 | 329 |
| FAP CAR 6 | 330 |
| FAP binding domain 7 | 331 |
| FAP CAR 5 | 332 |
| FAP CAR 6 | 333 |
| FAP binding domain 8 | 334 |
| FAP CAR 5 | 335 |
| FAP CAR 6 | 336 |
| FCRL1 antigen | 337 |

### Example 2. In Vitro and In Vivo Assay: ALPP CAR T Cells

Alkaline phosphatase (placental type, ALPP for short) is a single transmembrane glycoprotein with 535 amino acids, which can catalyze the hydrolysis of phosphate monoesters. ALPP is mainly expressed in the placenta. In normal tissues, it is only expressed in the endometrium at a low level. However, ALPP is highly expressed in a variety of cancers including melanoma, seminoma, mesothelioma, gastric cancer, pancreatic cancer, ovarian cancer and endometrial cancer.

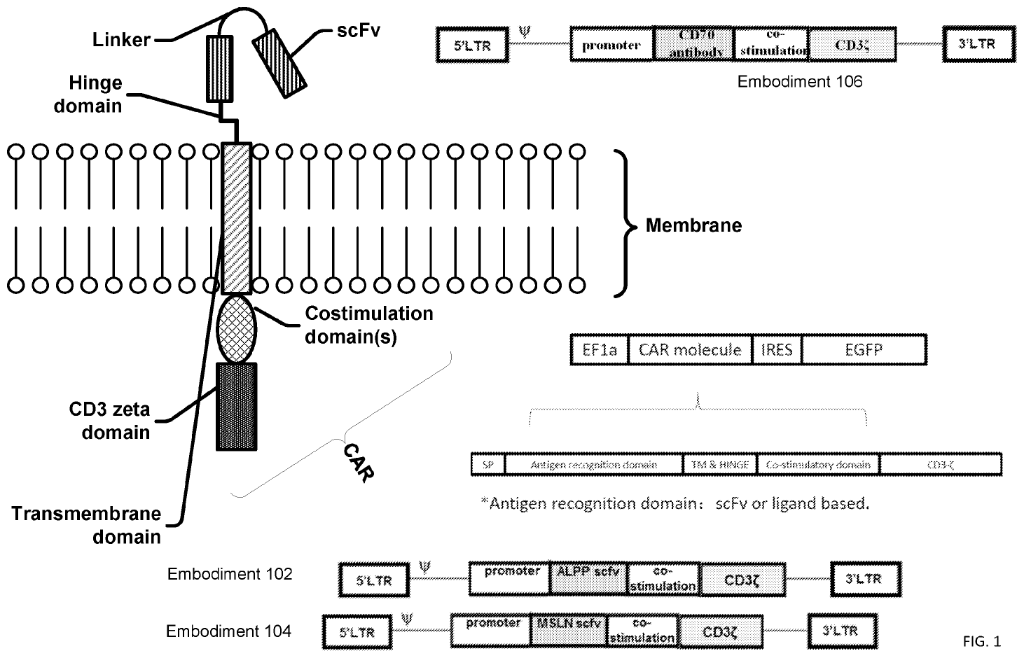

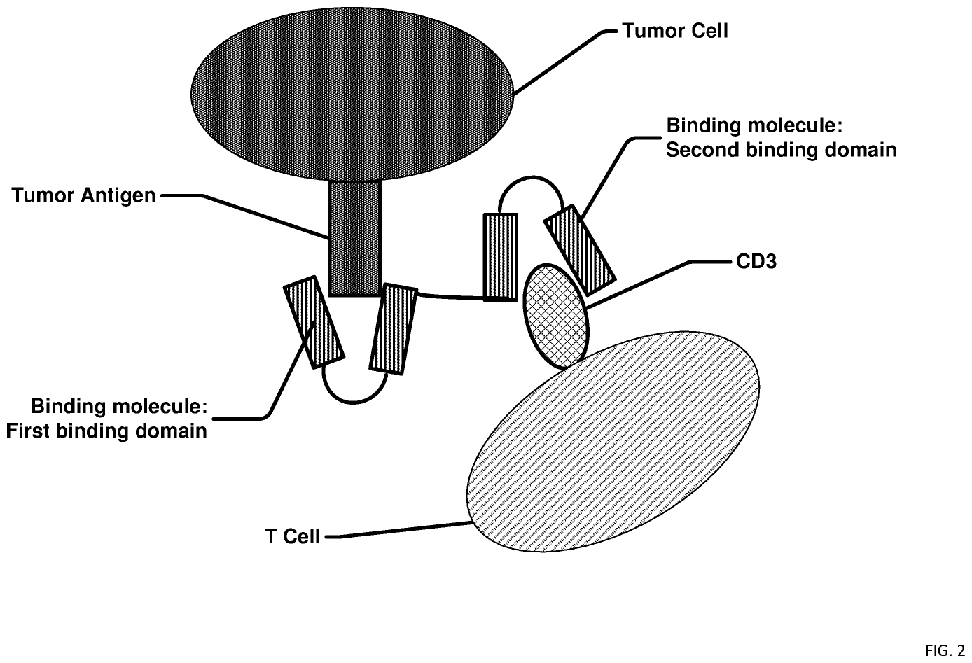

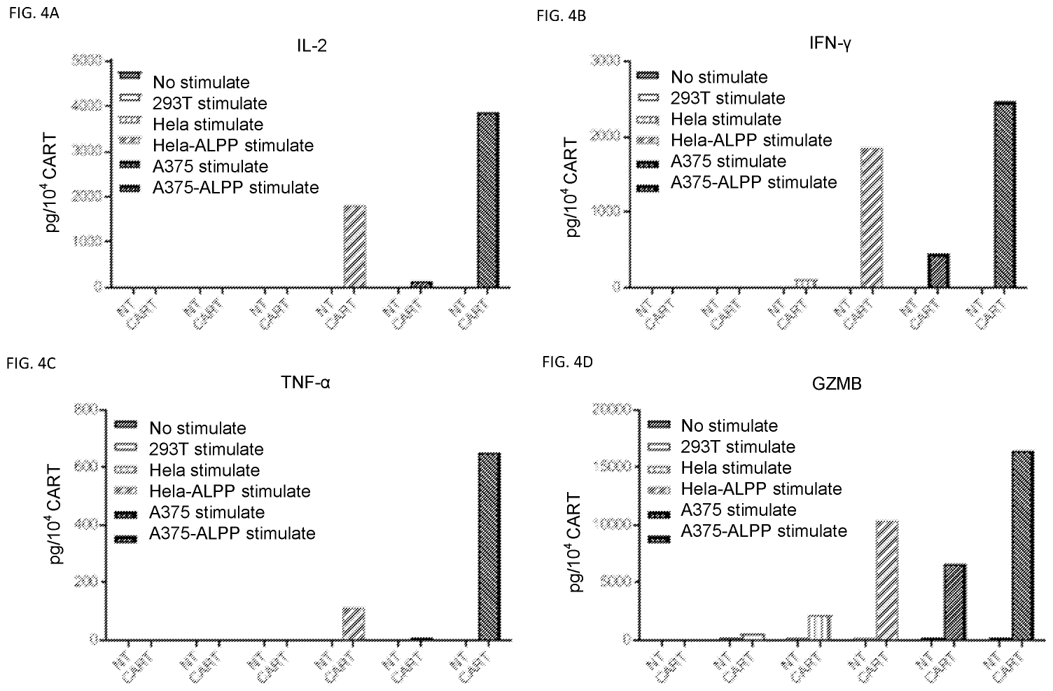

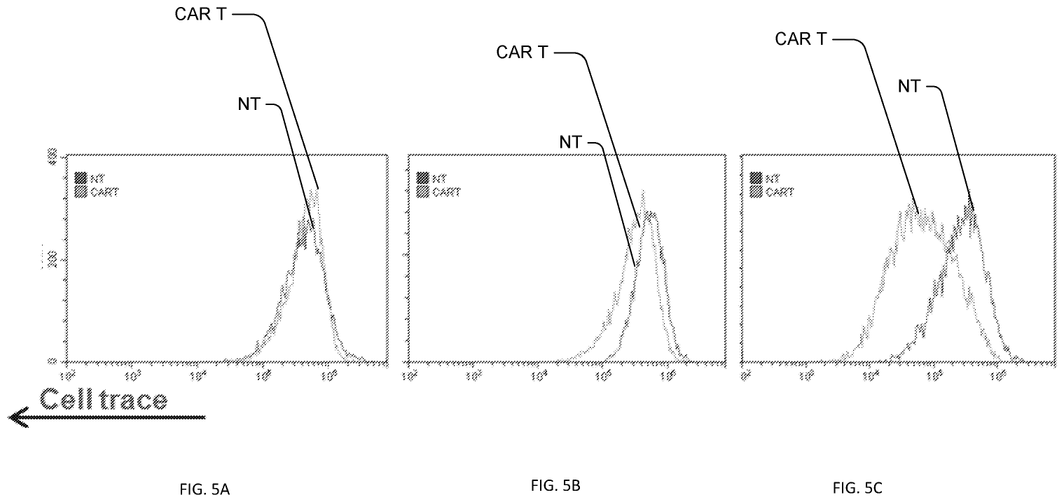

FIGS. 4A-4D, and 5A-5C show ALPP CAR T cells' anti-tumor activities in vitro experiments. Embodiment 102 of FIG. 1 shows a schematic diagram of the vector which includes the ALPP scFv sequence (SEQ ID NO: 42), transmembrane region, co-stimulatory domain, and CD3ζ. FIG. 4 shows cytokine release of ALPP CAR T cells cultured with various substrate cells. Cytometric Bead Array (CBA) was performed to detect IL2, TNF-α, IFN-γ, and Gramzyme B in the supernatant of the media. FIG. 5 shows cell proliferation of ALPP CAR T cells cultured with various substrate cells. Cell tracing assay (CellTracing™) was performed to observe the proliferation of the cells.

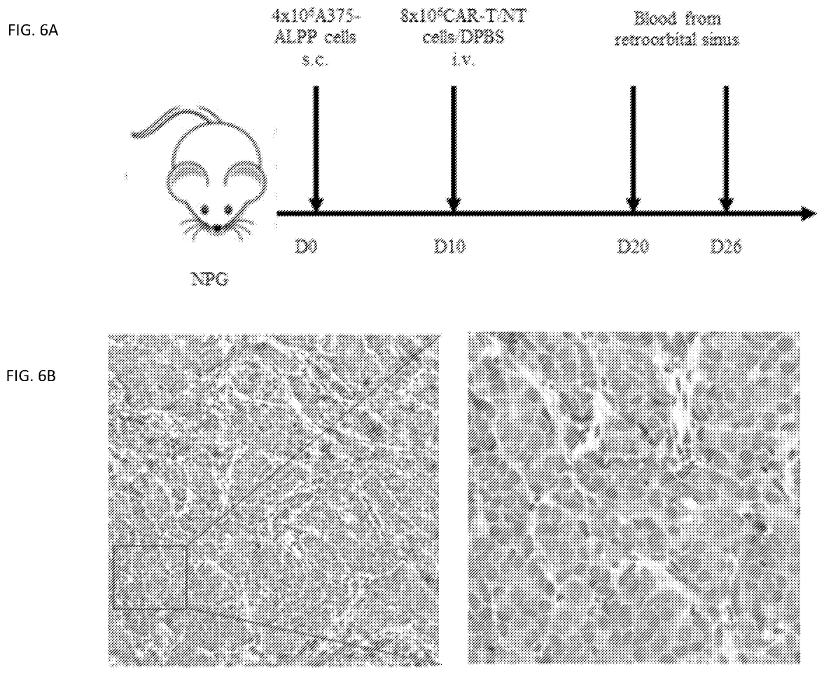

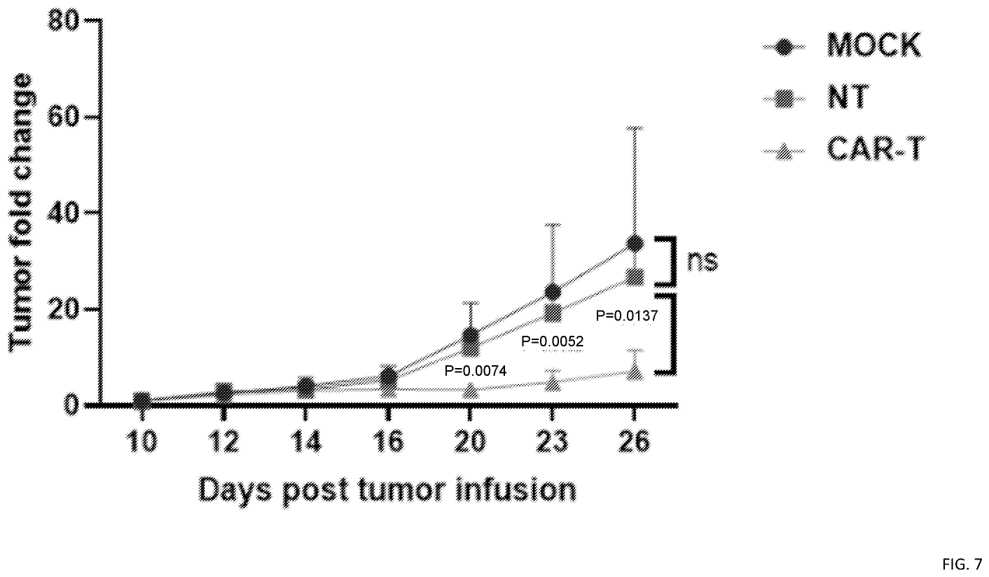

FIGS. 6 and 7 show ALPP CAR T cells have anti-tumor activities in in vivo experiments. Subcutaneous tumor model was constructed by subcutaneously injecting 4.0×10⁶ A375-ALPP cells into mice on day 1. On day 10, the mice inoculated with tumor cells were randomly divided into Mock group infused with no T cells, NT group infused with non-transduced T cells, and CAR T group infused with ALPP CAR T cells (SEQ ID NO: 43). These three groups of mice were given an equal volume of DPBS, 8.0×10⁶ NT cells, and 8.0×10⁶ ALPP CAR T cells through the tail vein, respectively. The body weight of the mice was recorded daily; the size of the tumor was measured; and blood was collected from the marginal venous plexus every week for flow cytometry to detect the proliferation of T cells in the peripheral blood.

FIG. 6A shows the scheme for in vivo experiments. FIG. 6B shows the use of immunohistochemical staining (Immunohistochemical staining, IHC) to detect the expression of ALPP in mice with subcutaneous tumors. (Antibody: R&D, Inc. Human Alkaline Phosphatase/ALPP Antibody Monoclonal Mouse IgG1 Clone #696128). FIG. 7 shows the changes in tumor volume of mice in each group over time. These results show that ALPP CAR T cells exhibited anti-tumor activities. After cell infusion, blood was taken from the marginal venous plexus of the mice for flow cytometry assay. The flow cytometry assay results showed that ALPP CAR T cells in the peripheral blood of the CAR T group were significantly proliferated. At day 26, continuous growth of subcutaneous tumors in the mock group and NT group was observed, while the growth of subcutaneous tumors in the CAR T group was inhibited. In sum, ALPP CAR T cells expanded in tumor-bearing mice and effectively inhibited the growth of tumors, showing strong anti-tumor activities with little toxicity.

### Example 3. In Vitro and In Vivo Assay: MSLN CAR T Cells

Mesothelin (MSLN) is a cell surface protein fixed by glycosylphosphatidylinositol and mainly plays a role of adhesion. MSLN is expressed in normal mesothelial cells and a wide range of high expression in solid tumors including mesothelioma, breast cancer, pancreatic cancer, and lung cancer.

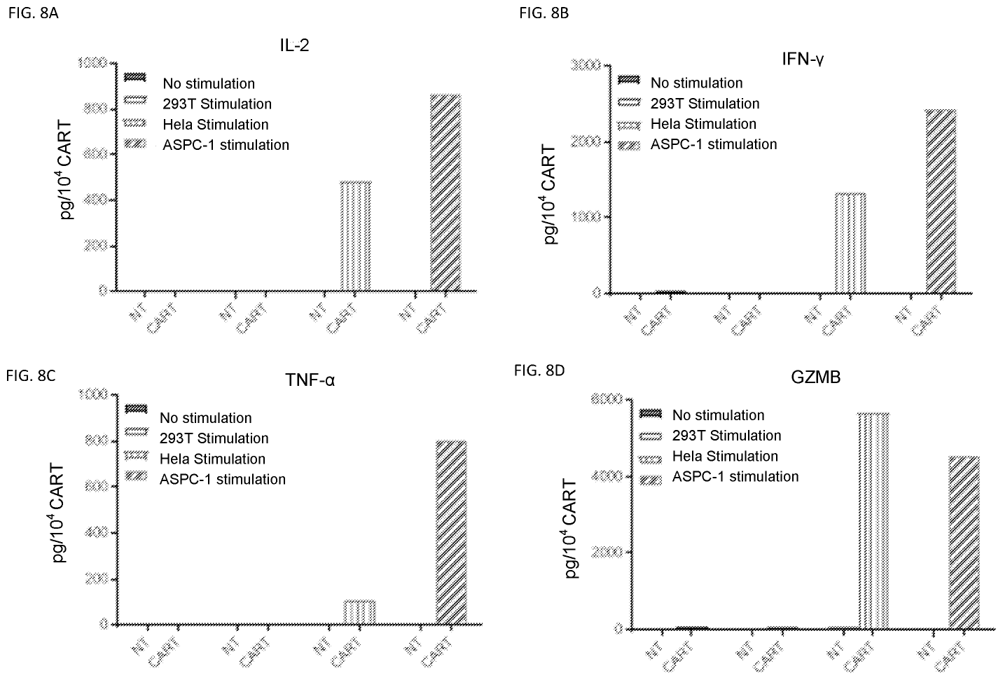

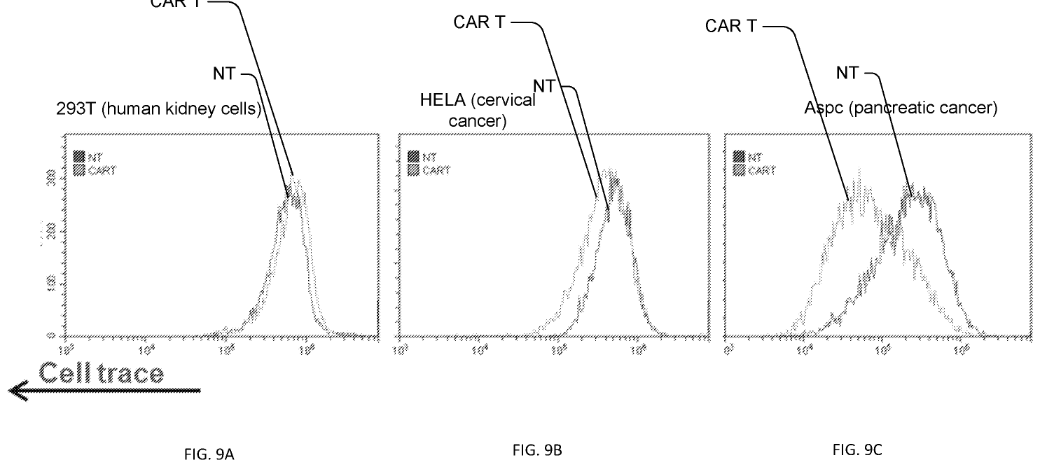

FIGS. 8A-8D, and 9A-9C show MSLN CAR T cells' anti-tumor activities in in vitro experiments. Embodiment 104 of FIG. 1 shows a schematic diagram of the vector which includes the MSLN scFv sequence (SEQ ID NO: 23), transmembrane region, co-stimulatory domain, and CD3. FIGS. 8A-8D show cytokine release of MSLN CAR T cells cultured with various substrate cells. Cytometric Bead Array (CBA) was performed to detect IL2, TNF-α, IFN-γ, and Gramzyme B in the supernatant of the media. FIGS. 9A-9C show cell proliferation of MSLN CAR T cells cultured with various substrate cells. Cell tracing assay (CellTracing™) was performed to observe the proliferation of cells.

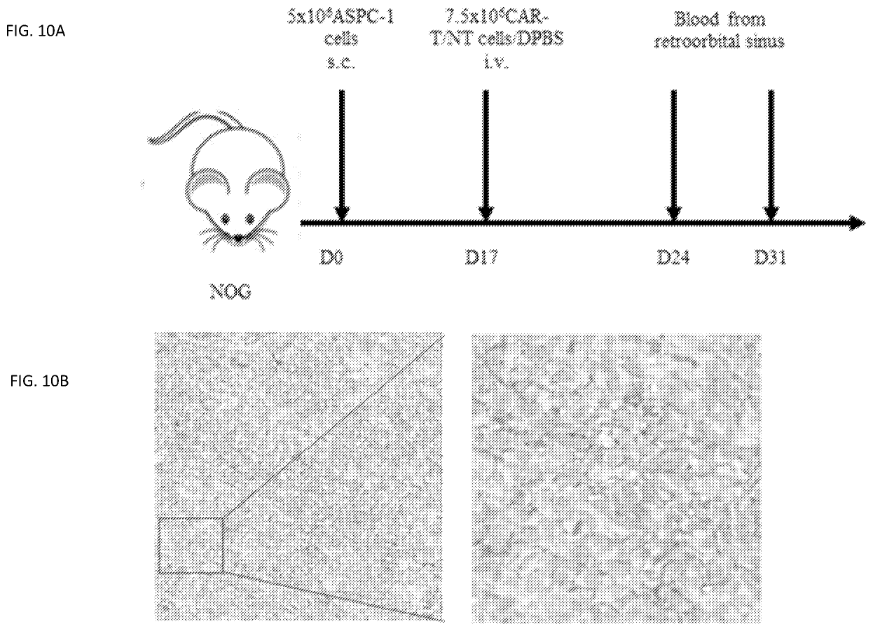

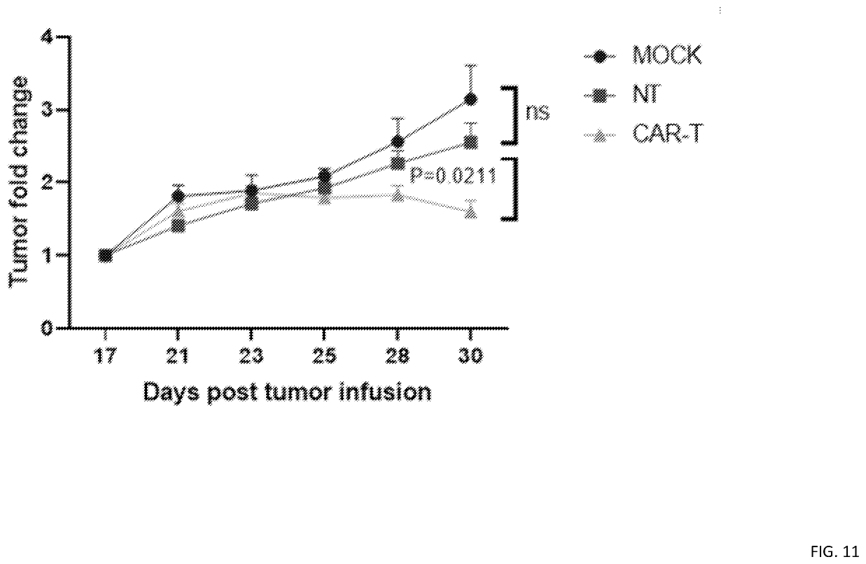

FIGS. 10A-10B, and 11 show MSLN CAR T cells have anti-tumor activities in in vivo experiments. Subcutaneous tumor model was constructed by subcutaneously injecting 4.0×10⁶ ASPC-1 cells into mice on day 1. On day 17, the mice inoculated with tumor cells were randomly divided into Mock group infused with no T cells, NT group infused with non-transduced T cells, and CAR T group infused with MSLN CAR T cells (SEQ ID NO: 24). These three groups of mice were given an equal volume of DPBS, 7.5×10⁶ NT cells, and 7.5×10⁶ MSLN CAR T cells through the tail vein, respectively. The body weight of the mice was recorded daily; the size of the tumor was measured; and blood was collected from the marginal venous plexus every week for flow cytometry to detect the proliferation of T cells in the peripheral blood.

FIG. 10A shows the scheme for in vivo experiments. FIG. 10B shows the use of immunohistochemical staining (Immunohistochemical staining, IHC) to detect the expression of MSLN in mouse with subcutaneous tumors. (Antibody: Abcam, Inc. Recombinant Anti-Mesothelin Monoclonal Rabbit IgG Clone #EPR19025-42). FIG. 11 shows the changes in tumor volume of mice in each group over time. These results show that MSLN CAR T cells exhibited anti-tumor activities. After cell infusion, blood was taken from the marginal venous plexus of the mice for flow cytometry assay. The flow cytometry assay results showed that MSLN CAR T cells in the peripheral blood of the CAR T group were significantly expanded. On day 30, continuous growth of subcutaneous tumors in the mock group and NT group was observed, while the growth of subcutaneous tumors in the CAR T group was inhibited. In sum, MSLN CAR T cells expanded in tumor-bearing mice and effectively inhibited the growth of tumors, showing strong anti-tumor activities with little toxicity.

### Example 4. In Vitro and In Vivo Assay: CD70 CAR T Cells

CD70 (Tumor necrosis factor ligand superfamily member 7, TNFSF7), a member of the tumor necrosis factor (TNF) superfamily, is also a ligand for CD27. CD70 is expressed at low levels in some B cells and dendritic cells and is not expressed or expressed at very low levels in some important organs. Because of its high expression in tumors such as kidney cancer, pancreatic cancer, lung cancer and melanoma, it has become a target of tumor immunotherapy.

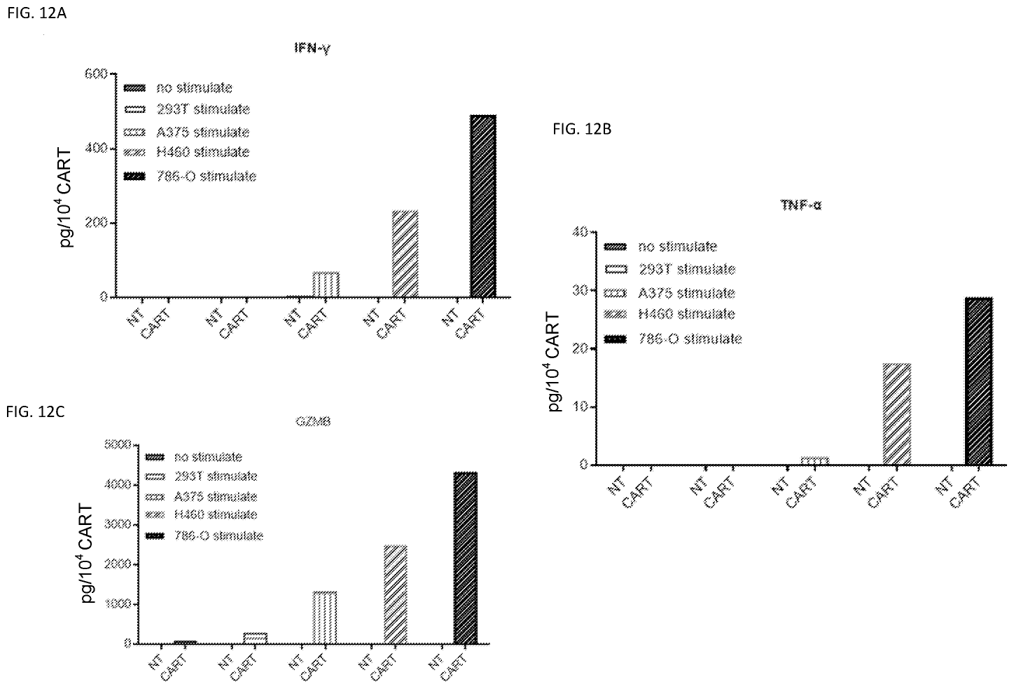

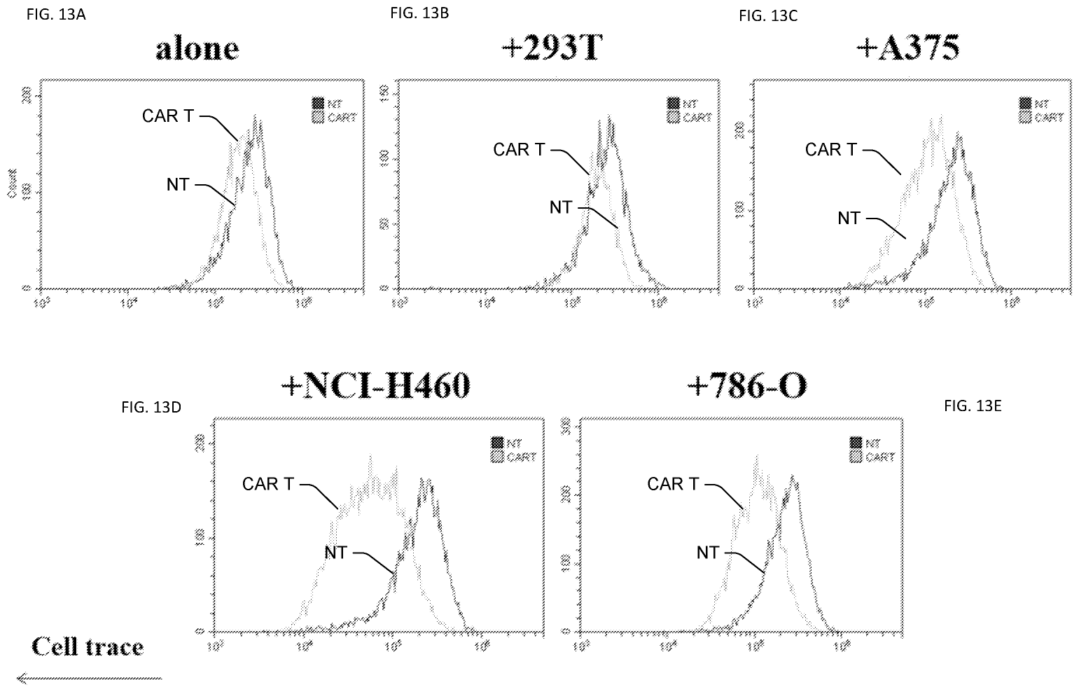

FIGS. 12A-12C, and 13A-13E show MSLN CAR T cells' anti-tumor activities in vitro experiments. Embodiment 106 of FIG. 1 shows a schematic diagram of the vector which includes the CD70 scFv sequence (SEQ ID NO: 2), transmembrane region, co-stimulatory domain, and CD3. FIGS. 12A-12C show cytokine release by CD70 CAR T cells cultured with various substrate cells. Cytometric Bead Array (CBA) was performed to detect TNF-α, IFN-γ, and Gramzyme B in the supernatant of the media. FIGS. 13A-13E show cell proliferation of CD70 CAR T cells cultured with various substrate cells. Cell tracing assay (CellTracing™) was performed to observe the proliferation of cells.

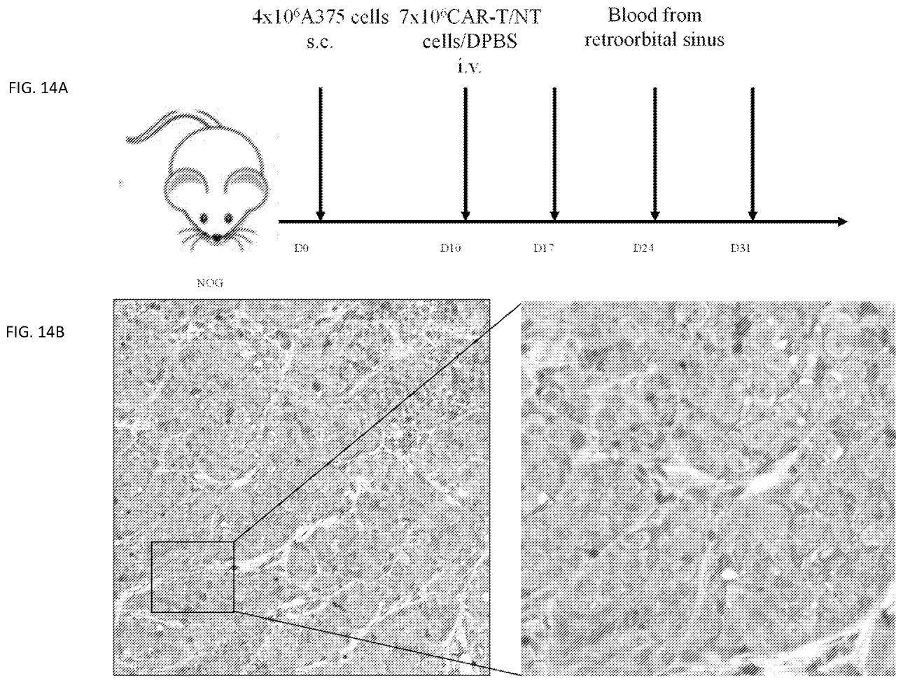

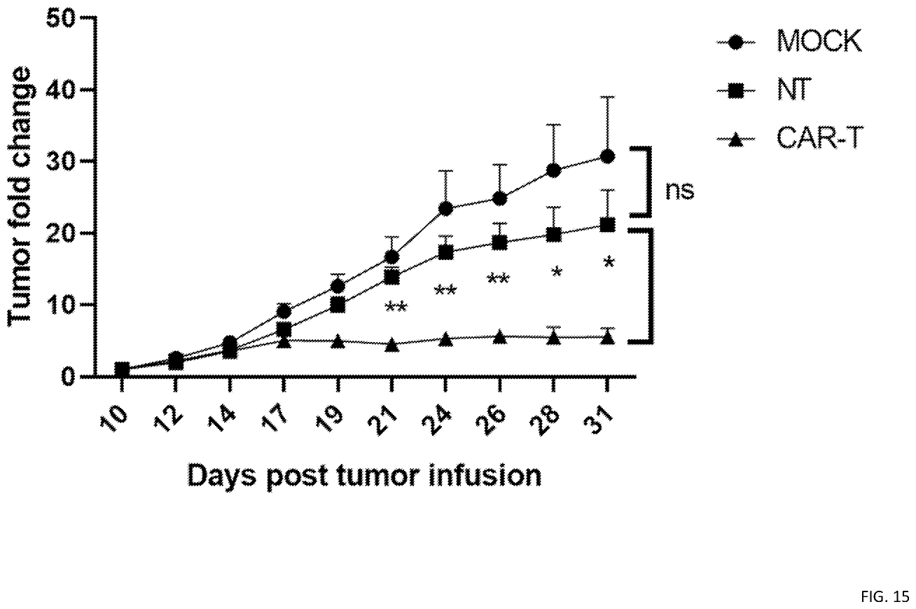

FIGS. 14A-14B, and 15 show CD70 CAR T cells had anti-tumor activities in in vivo experiments. Subcutaneous tumor model was constructed by subcutaneously injecting 4.0×10⁶ A375 cells into mice on day 1. On day 17, the mice inoculated with tumor cells were randomly divided into Mock group infused with no T cells, NT group infused with non-transduced T cells, and CAR T group infused with CD70 CAR T cells (SEQ ID NO: 3). These three groups of mice were given an equal volume of DPBS, 7.5×10⁶ NT cells, and 7.5×10⁶ CD70 CAR T cells through the tail vein, respectively. The body weight of the mice was recorded daily; the size of the tumor was measured; and blood was collected from the marginal venous plexus every week for flow cytometry to detect the proliferation of T cells in the peripheral blood.

FIG. 14A shows the scheme for in vivo experiments. FIG. 14B shows the use of immunohistochemical staining (Immunohistochemical staining, IHC) to detect the expression of CD70 in mice with subcutaneous tumors. (R&D, Inc. Human CD27 Ligand/TNFSF7 Antibody Monoclonal Mouse IgG2B Clone #301731). FIG. 15 shows the changes in tumor volume of mice in each group over time. These results show that CD70 CAR T cells exhibited anti-tumor activities. After cell infusion, blood was taken from the marginal venous plexus of the mice for flow cytometry assay. The flow cytometry assay results showed that CD70 CAR T cells in the peripheral blood of the CAR T group were significantly expanded. On day 30, continuous growth of subcutaneous tumors in the mock group and NT group was observed, while the growth of subcutaneous tumors in the CAR T group was inhibited. In sum, CD70 CAR T cells expanded in tumor-bearing mice and effectively inhibited the growth of tumors, showing strong anti-tumor activities with little toxicity.

### Example 5. In Vitro and In Vivo Assay: ACPP CAR T Cells

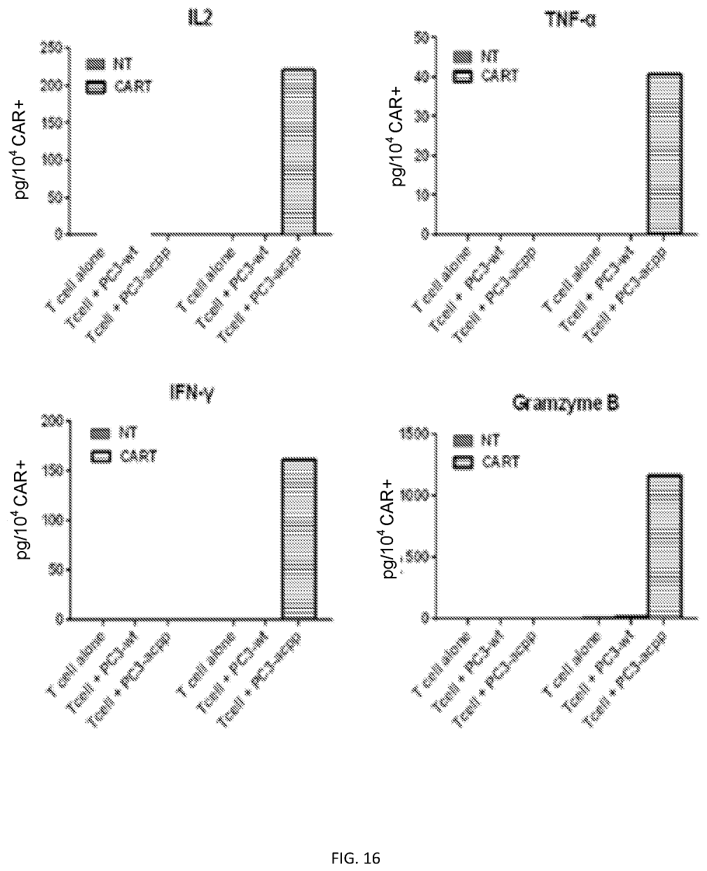

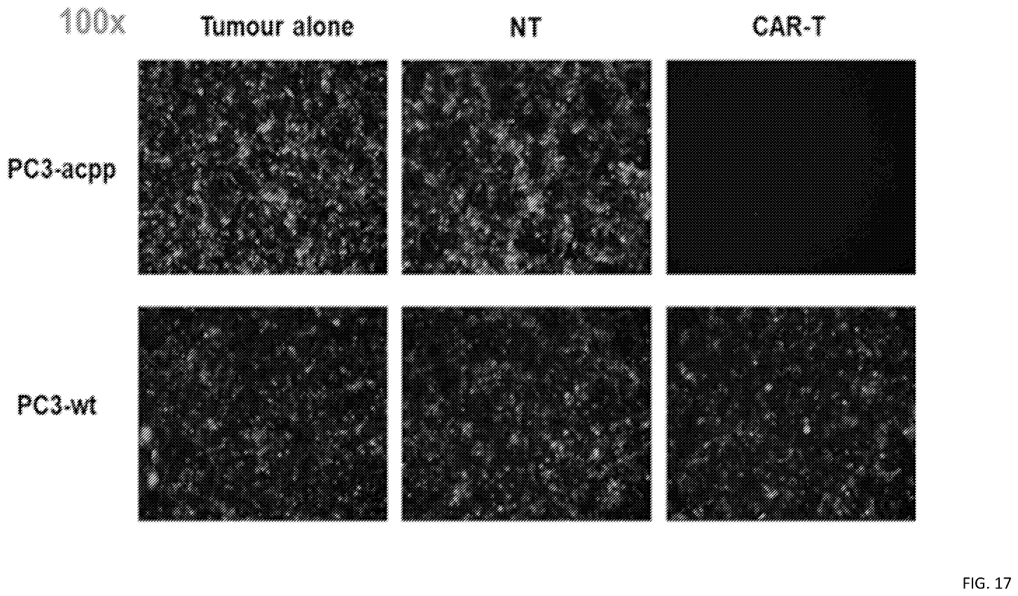

FIGS. 16 and 17 show ACPP CAR T cells exhibited good anti-tumor activity in vitro. The expression of ACPP on the surface of tumor cells PC3-WT and PC3-ACPP was determined using flow cytometry. (The expression of CD137, T cell activation marker, was detected 24 hours after co-culturing CAR T cells or NT cells with PC3-ACPP using flow cytometry. Determination of IL-2, TNF-α, IFN-γ, and Granzyme B in the supernatant of the cultures 24 hours after co-culturing CAR T cells or NT cells with PC3-wt or PC3-ACPP using Cytometric Bead Array (CBA). Fluorescence imaging results of tumor cells alone or five days after co-culture with CAR T cells or NT cells (100×). The results of the in vitro studies show that ACPP CAR T cells (SEQ ID NOs: 49 and 50) can be effectively activated and can release multiple cytokines after contact with ACPP-positive tumor cells, to specifically kill tumor cells. When ACPP CAR T cells were co-cultured with PC3-ACPP cells, ACPP CAR T cells were specifically activated compared to NT cells, showing increased CD137 expression. High levels of IL-2, TNF-α, IFN-γ and Granzyme B were detected in the supernatant of the cultures by flow cytometry after 24-hour. In addition, PC3-ACPP and PC3-wt were both labeled with green fluorescent protein, and the cytotoxicity effect of CAR T cells on tumor cells was observed under fluorescence microscope. It was found that CAR T cells can effectively and specifically kill PC3-ACPP cells, but had no observable cytotoxic effect on PC3-wt.

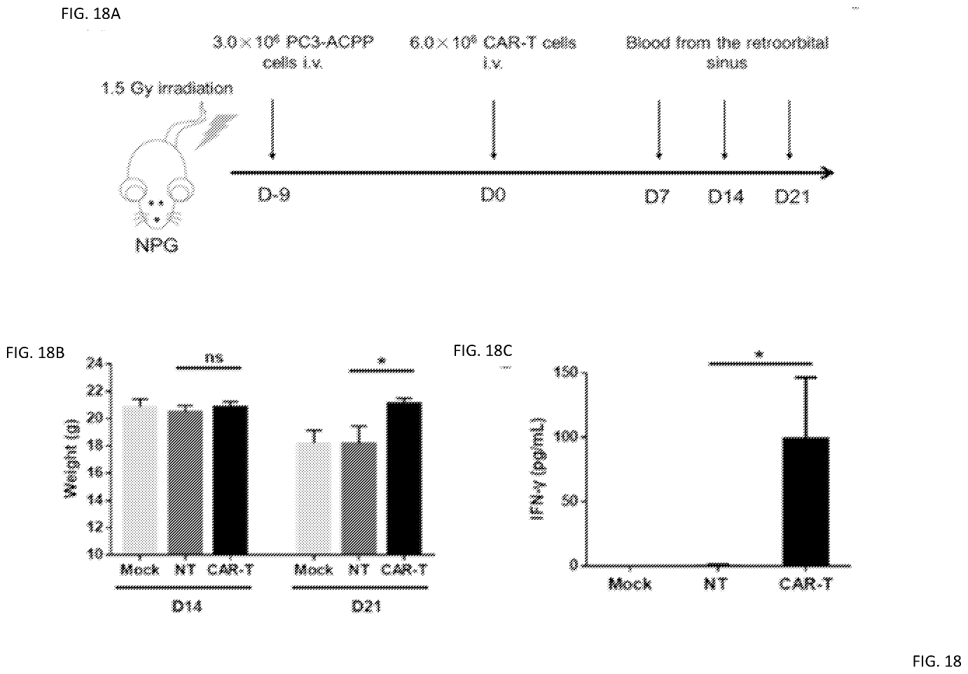

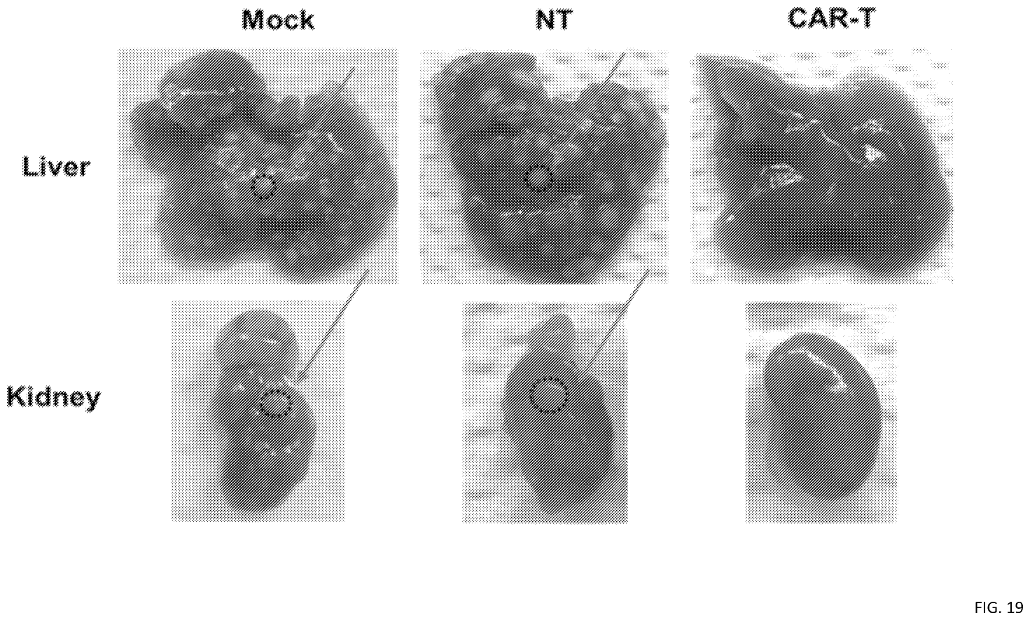

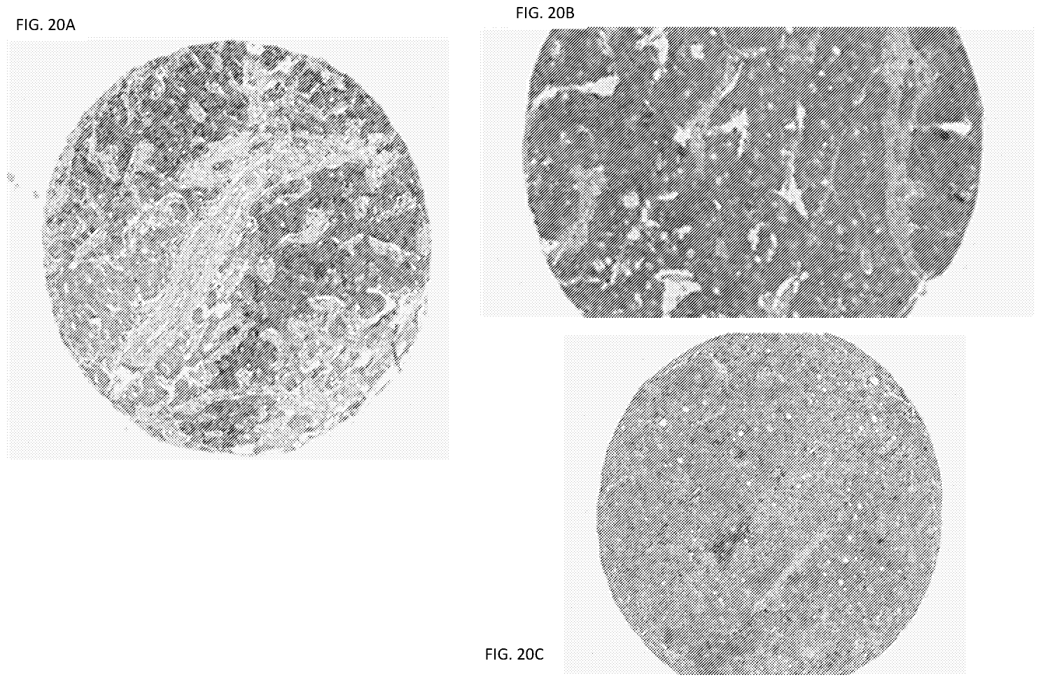

FIGS. 18A-18C and 19 show that ACPP CAR T cells exhibited good anti-tumor effect in mice. Scheme for in vivo experiments: Prostate cancer metastatic model was established by injecting PC3-ACPP tumor cells through the caudal vein of mice on day −9 (D-9), followed by injecting CAR T cells or NT cells 9 days later. Peripheral blood was collected at D7, D14 and D21 for analysis. Changes in body weight of mice in each group at D14 and D21. The level of IFN-γ in the peripheral blood of mice at D7. The absolute number of human T cells and the ratio of human T cells to mouse white blood cells in the peripheral blood at D7, D14 and D21. Tumor metastasis in liver and kidney of each group. *: P<0.05, **: P<0.01, ***: P<0.001. In vivo studies show that ACPP CAR T cells exhibited good anti-tumor effect. Body weight monitoring revealed significant weight loss at D21 in mice in both MOCK and NT groups. Peripheral blood was collected every week after infusion. Significant human T cell proliferation along with IFN-γ release was observed in the peripheral blood of mice in the CAR T group. By D21, the mice in both MOCK and NT group were observed to have dark and rough hair, asthenia, paralysis, hunchback and even death, while the mice in the CAR T group were all in healthy condition. The mice were sacrificed on D21, and their main organs were collected and analyzed for tumor metastasis. It was found that the ACPP CAR T cells significantly inhibited liver and kidney metastasis of PC3-ACPP tumor cells. In conclusion, ACPP CAR T cells can significantly proliferate in vivo, effectively inhibit tumor metastasis, and show strong anti-tumor activity. Additionally, and no significant damage was observed in the major organs of the mice in the CAR T group, indicating a specific cytotoxicity to ACPP tumor cells.

### Example 6. In Vitro and In Vivo Assay: CLDN6 CAR T Cells

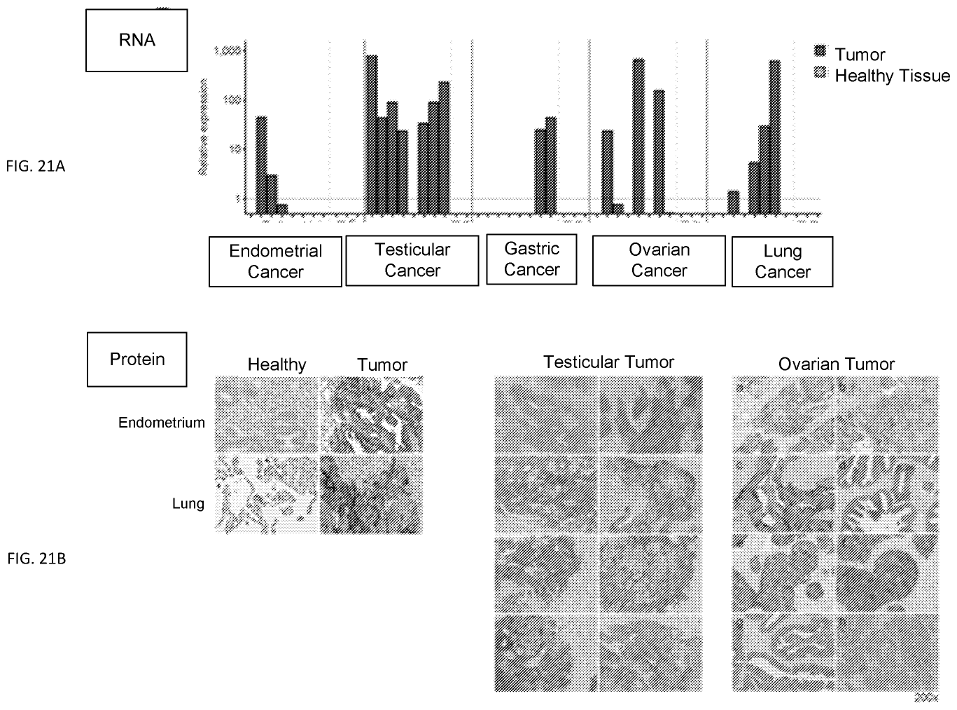

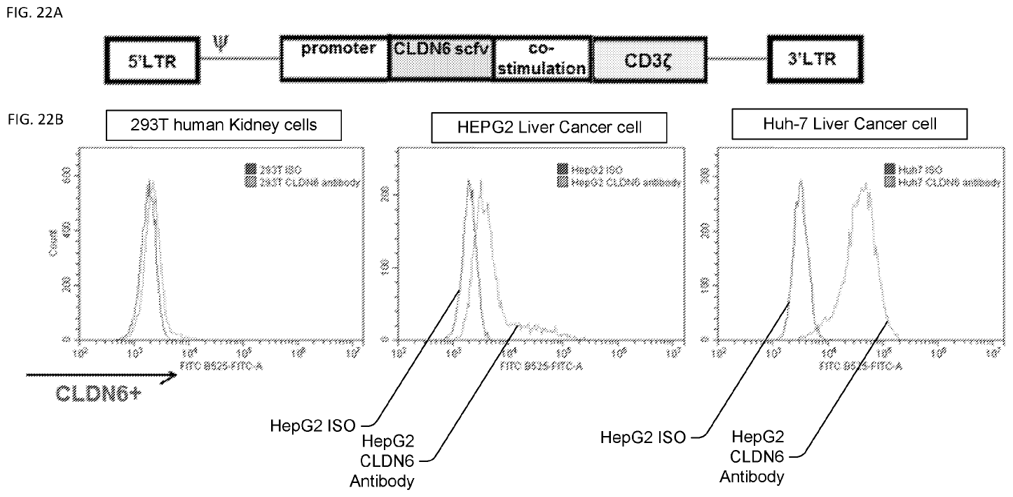

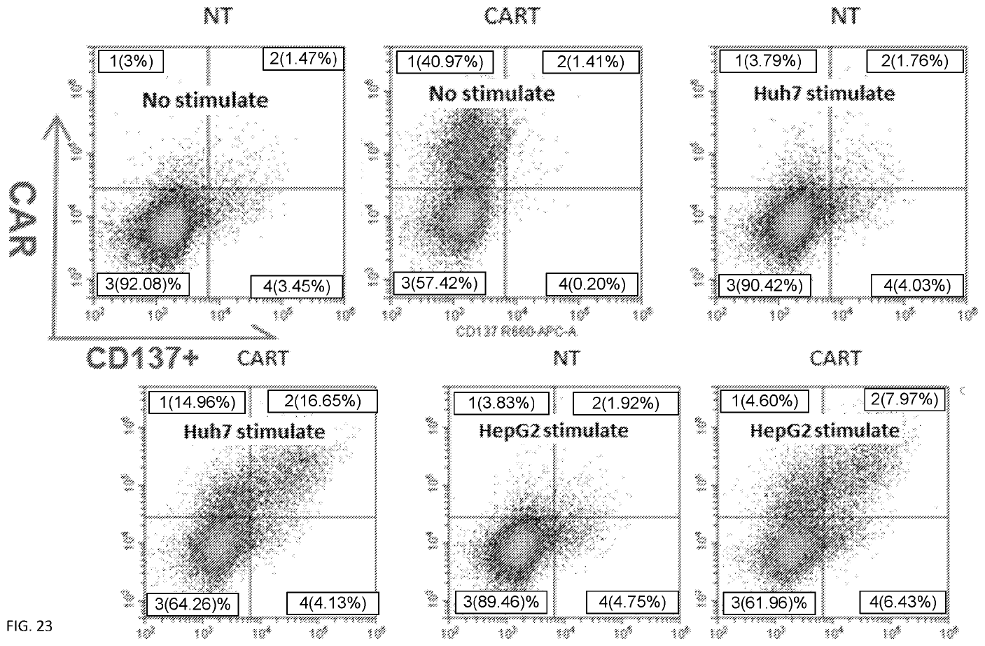

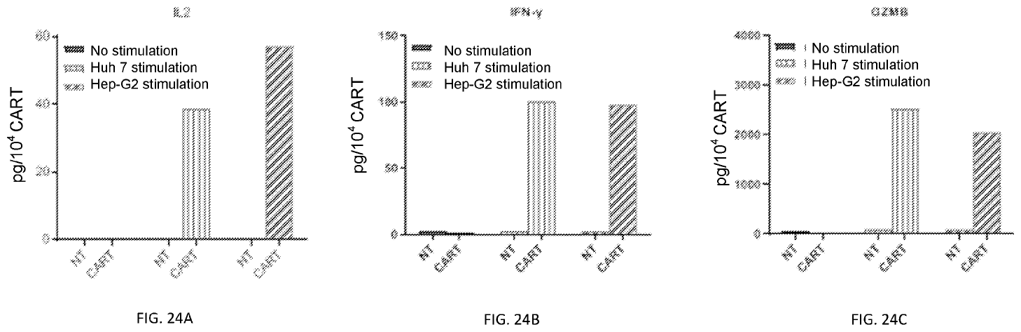

FIGS. 21A-21B show RNA and protein levels of CLDN6 expression in tumor tissues. FIGS. 22A-22B, 23, and 24A-24C show CLDN6 CAR T cells exhibited good anti-tumor activity in vitro. FIG. 22A shows the construct of the vector encoding CLDN6 CAR (scFv of CLDN6 CAR: SEQ ID NO: 12; CLDN6 CAR: SEQ ID NO: 14). FIG. 22B shows the expression of CLDN6 on the surface of HEPG2, and Huh-7, and CLDN6 expression was determined using CLDN6 antibodies. FIG. 23 shows the expression of CD137, T cell activation marker, was detected 24 hours after co-culturing CAR T cells or NT cells with HepG2 or Huh7 using flow cytometry. FIGS. 24A-24C show determination of IL-2, IFN-γ, and Granzyme B in the supernature of the cultures 24 hours after co-culturing CAR T cells or NT cells with PC3-wt or PC3-CLDN6 using Cytometric Bead Array (CBA). The results of the in vitro studies show that CLDN6 CAR T cells can be effectively activated and can release multiple cytokines after contact with CLDN6-positive tumor cells, to specifically kill tumor cells.

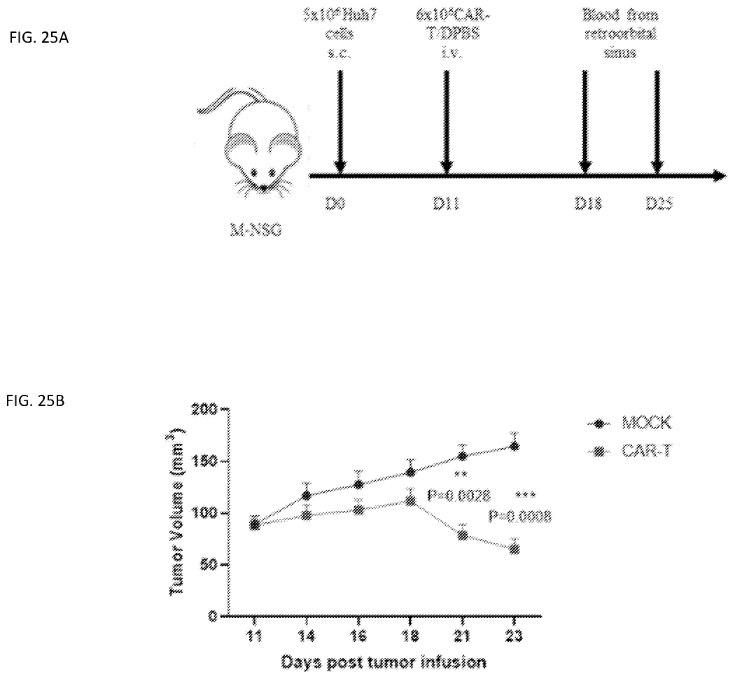

FIGS. 25A-25B show that CLDN6 CAR T cells exhibited good anti-tumor effect in mice. FIG. 25A shows the scheme for in vivo experiments: Liver cancer metastatic model was established by injecting Huh-7 tumor cells through the caudal vein, followed by injecting CAR T cells or NT cells 9 days later. Peripheral blood was collected on D11, D18 and D25 for analysis. FIG. 25B shows changes in tumor volume of mice infused with Mock T cells and mice infused with CLDN6 CAR T cells. The results of the in vivo studies show that CLDN6 CAR T cells exhibited good anti-tumor effect.

### Example 7. In Vitro and In Vivo Assay: GPC-3 CAR T Cells

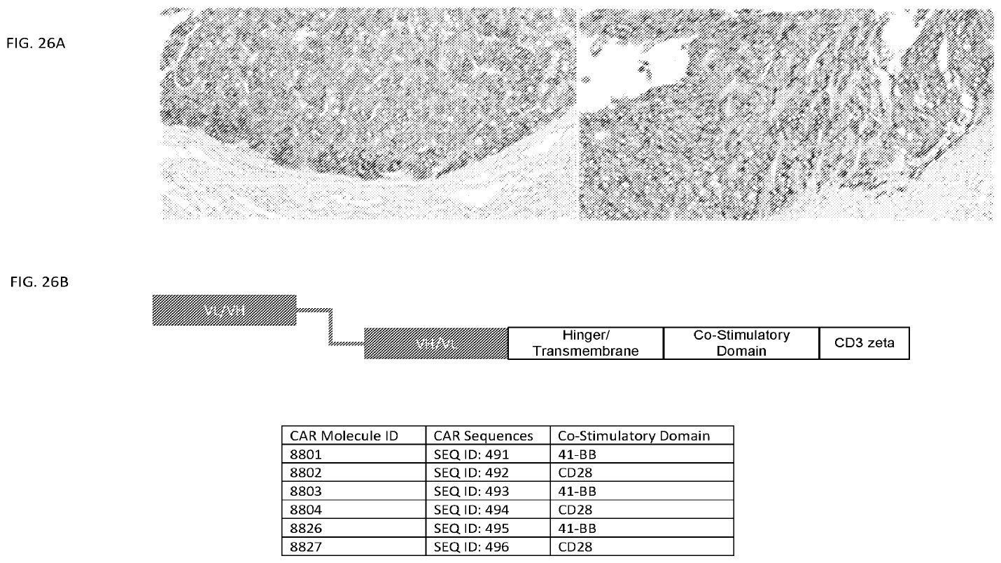

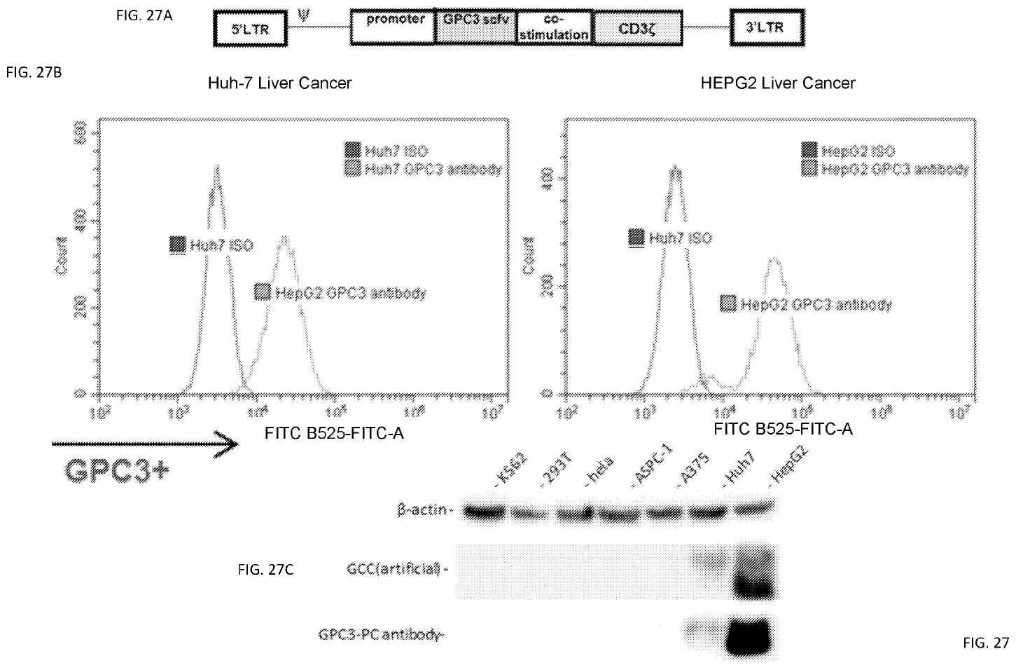

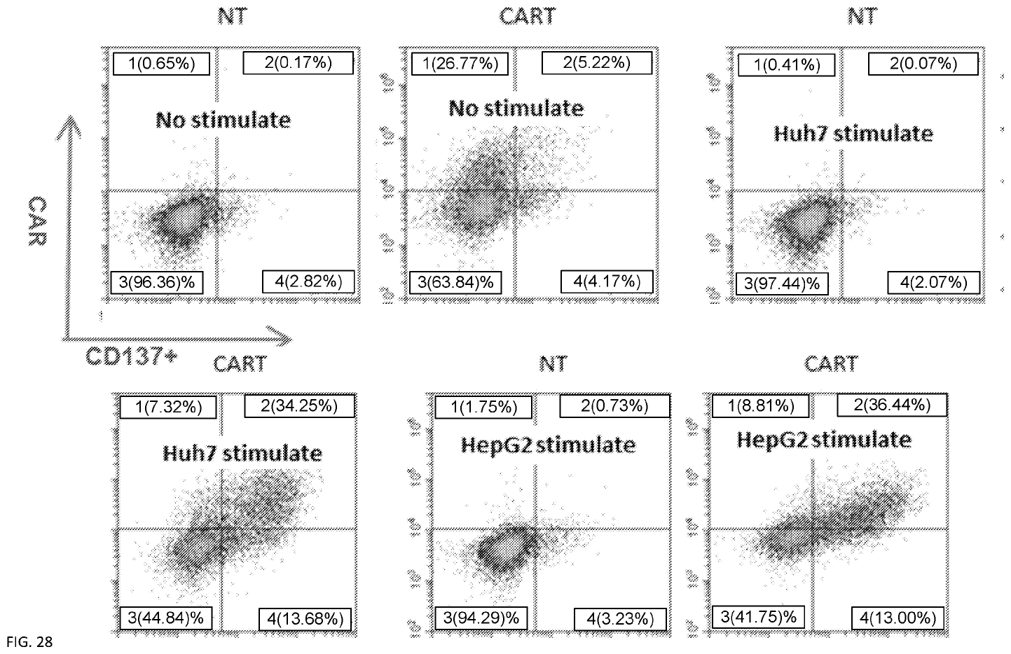

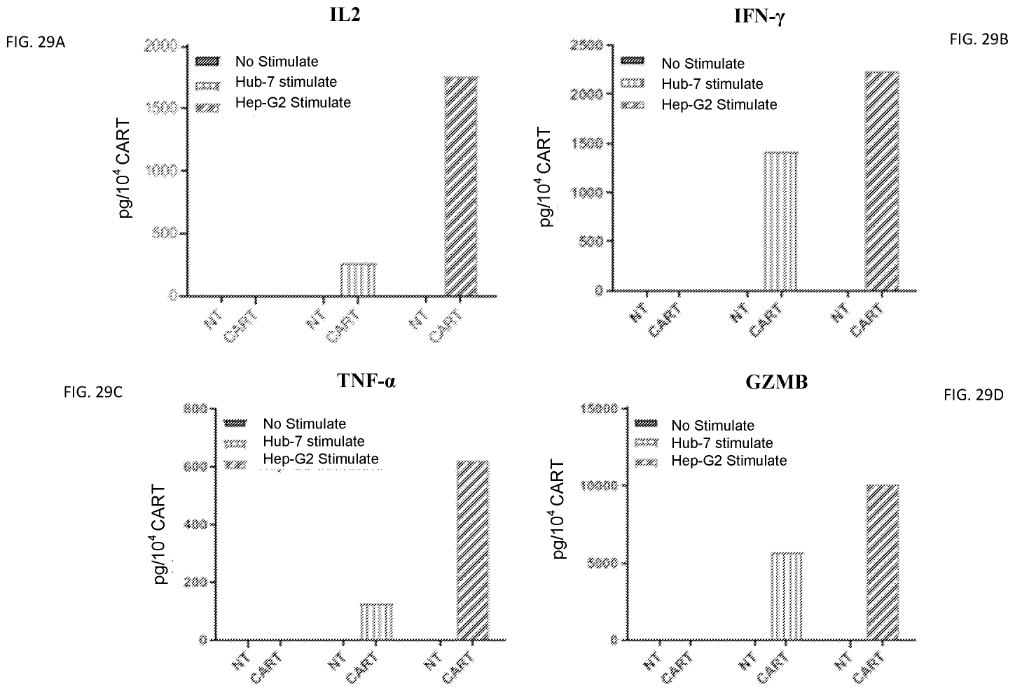

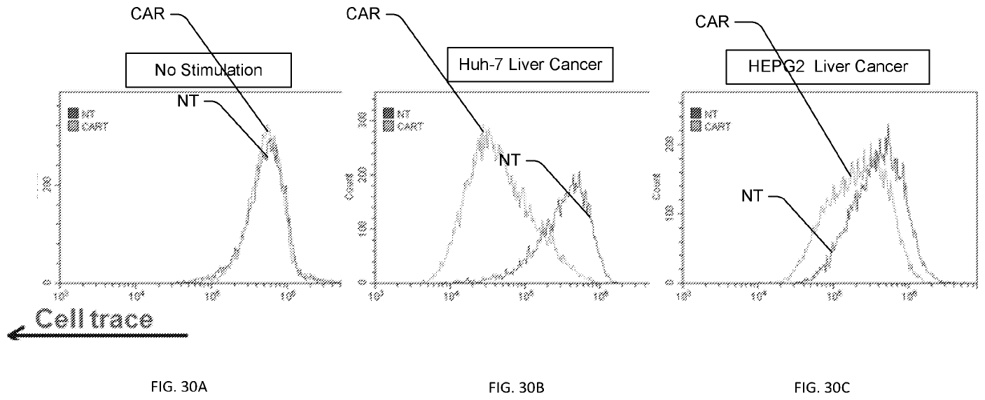

FIG. 26A shows GPC-3 expression in tumor tissues. FIG. 26B shows various structures of CAR molecules used for targeting GPC-3. Vectors encoding each of these CARs were transferred into T cells, to obtain various GPC-3 CAR T cells, and anti-tumor activities were observed when the CAR T cells were co-cultured with target cells expressing GPC-3. For example, FIGS. 27A-27C, 28, 29A-29D, and 30A-30C show GPC-3 CAR T cells (CAR Molecule ID 8803, scFv of GPC-3 CAR: SEQ ID NO: 16) exhibited good anti-tumor activity in vitro. FIG. 27A shows that the vector encoding GPC-3 CAR. FIG. 27B shows the expression of GPC-3 on the surface of HEPG2 and Huh-7, and GPC-3 was determined using GPC-3 antibodies. FIG. 27C shows western blot results of GPC-3 expression in various cells. FIG. 28 shows the expression of CD137, a T cell activation marker, was detected 24 hours after co-culturing CAR T cells or NT cells with or without HepG2 or with or without Huh7 using flow cytometry. FIGS. 29A-29D shows determination of IL-2, TNF-α, IFN-γ, and Granzyme B in the supernatant of the cultures 24 hours after co-culturing CAR T cells or NT cells with substrate cells. FIGS. 30A-30C show results of CellTrace™ assay showing proliferation of CAR T cells co-cultured with substrate cells. The results of the in vitro studies show that GPC-3 CAR T cells can be effectively activated and can release multiple cytokines after contact with GPC-3-positive tumor cells to specifically kill tumor cells.

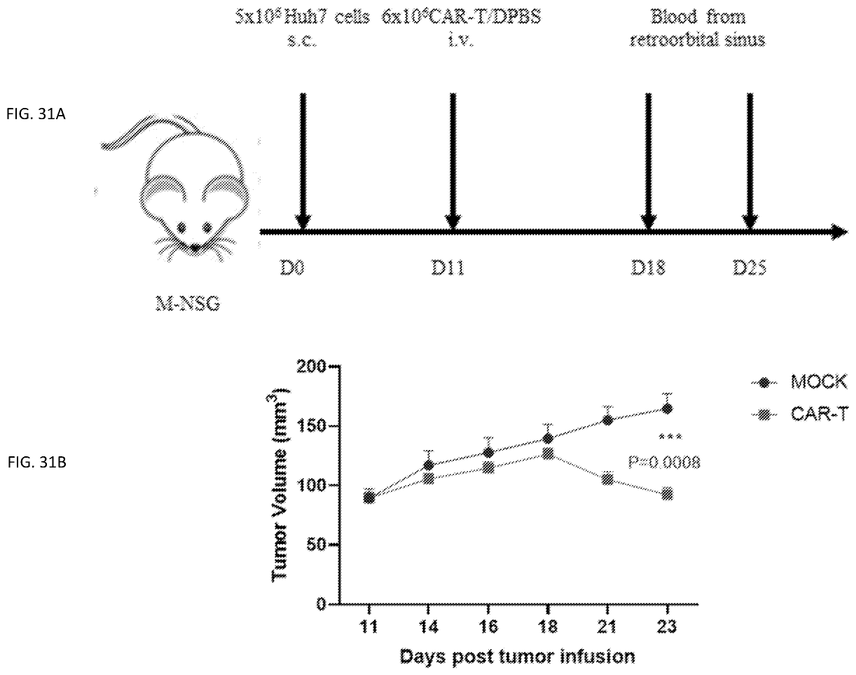

FIGS. 31A-31B show that GPC-3 CAR T cells (CAR Molecule ID 8803, GPC-3 CAR: SEQ ID NO: 17) exhibited good anti-tumor effect in mice. FIG. 31A shows the scheme for in vivo experiments. Metastatic liver cancer model was established by injecting Huh-7 tumor cells through the caudal vein of mice, followed by injecting CAR T cells or NT cells 9 days later. Peripheral blood was collected at D11, D18 and D25 for analysis. FIG. 31B shows changes in tumor volume of mice infused with Mock T cells and mice infused with GPC-3 CAR T cells. The results of the in vivo studies show that GPC-3 CAR T cells exhibited good anti-tumor effect.

### Example 8. In Vivo and In Vitro Data

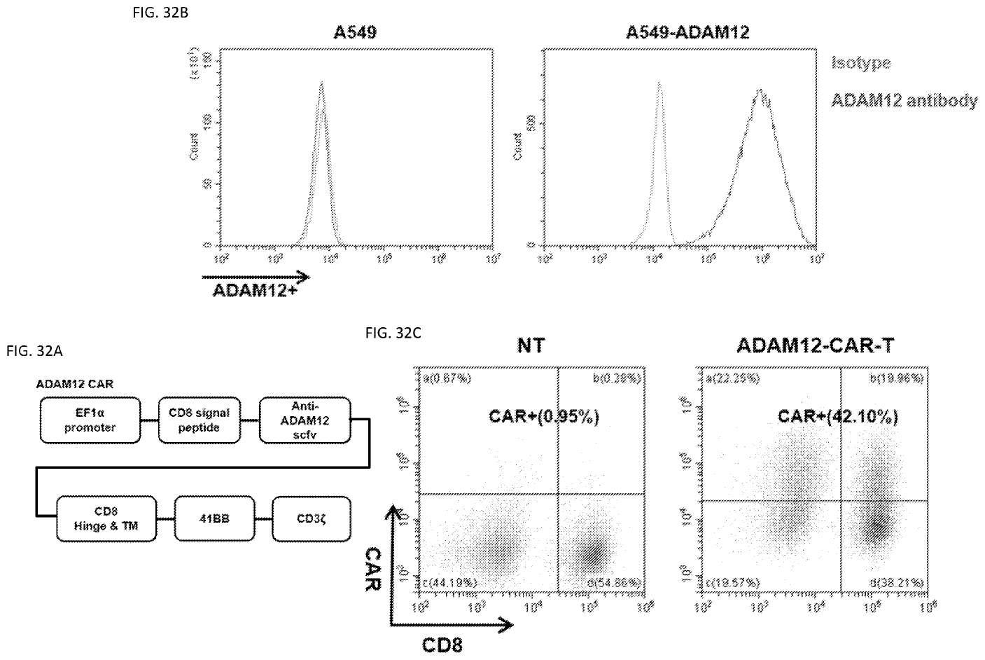

FIGS. 32A-32C. ADAM12 CAR T has good antitumor effect in vitro. (FIG. 32A) The figure shows the structure of the vector, including CD8 signal peptide, ADAM12 scFv sequence, transmembrane region, co-stimulatory domain and CD3ζ; (FIG. 32B) The expression of cell lines A549 and A549-ADAM12 was detected by flow cytometry; (FIG. 32C) The expression of NT and ADAM12-CAR T was detected by flow cytometry. Sequences of ADAM12 CAR and Anti-ADAM12 scFv are SED ID NOs: 56 and 55, respectively.

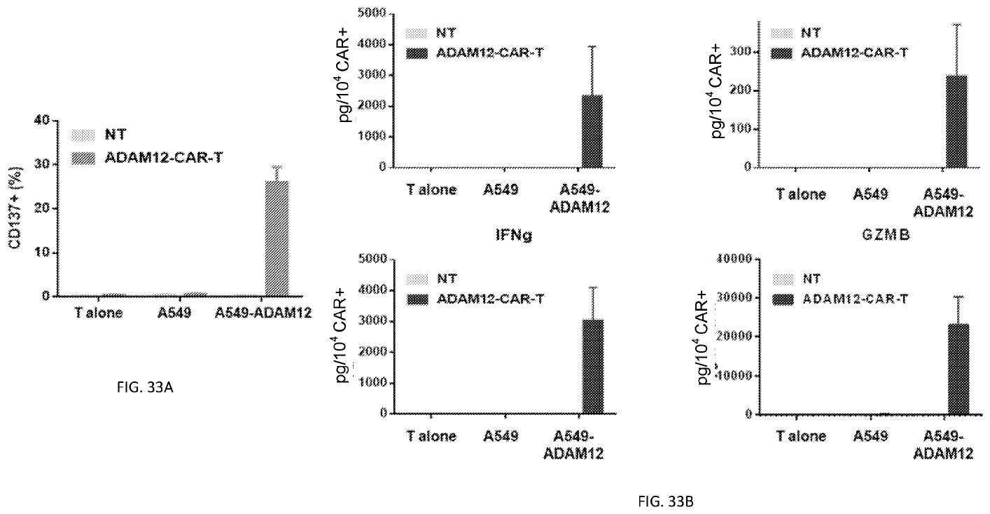

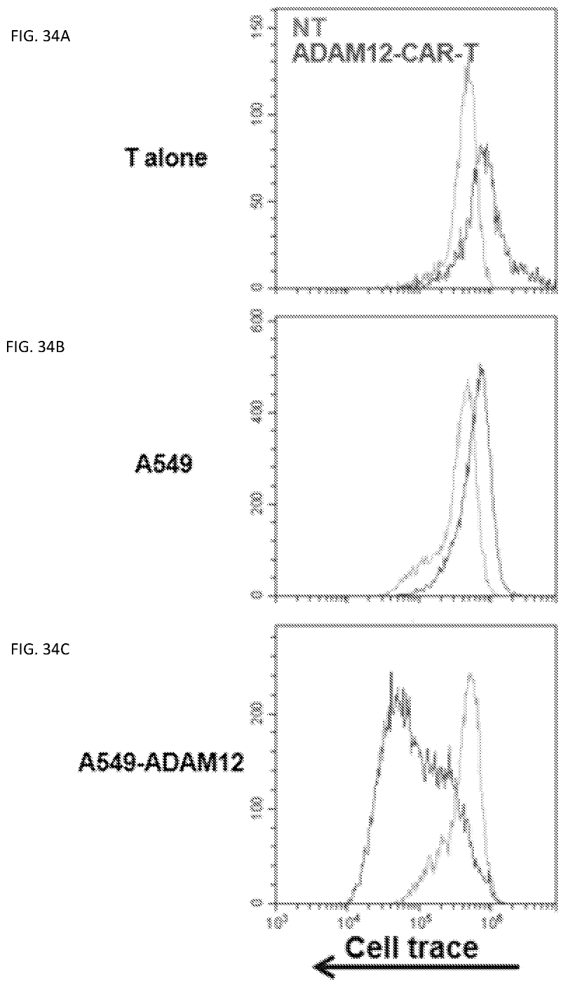

After tumor cell lines were co-cultured with CAR T cells for 24 hours, the expression of T cell activation marker CD137 was detected by flow cytometry (FIG. 33A). Cytometric Bear Array (CBA) was used to detect IL2, —TNF-α, IFN-γ, and Granzyme B in the supernatant of the cultures. The value is normalized and presented as pg per 10⁴ CAR T cells (FIG. 33B). After CAR T cells and NT cells were co-cultured with the above positive tumor cells, the proliferation of each group of cells was observed by CellTrace™ Cell Proliferation Kit (FIGS. 34A-34C).

The results of the in vitro experiments show that CAR T cells targeting ADAM12 can be effectively activated, and when cultured with positive tumor cells, these CAR T cells can release a variety of cytokines to specifically kill tumor cells and proliferate.

ADAM12 CAR T cells or control cells (non-transduced T cells) or PBS solution (mock) were injected (D0) into NOG mice with established tumors from lung cancer cell line A549-ADAM12 expressing ADAM12. Orbital sinus blood was then collected at D6, D13, and D21 post-injection to quantitate CAR T cells (by flow cytometry). Tumor volume was also measured at D-16, D0, and at 2-4 day intervals over 19 days. ADAM12 CAR T cells expanded in vivo up to 10⁵ cells/mL and with significant tumor elimination. The results show that ADAM12 CAR T cells can efficiently eliminate ADAM12-expressing tumor cells in vivo.

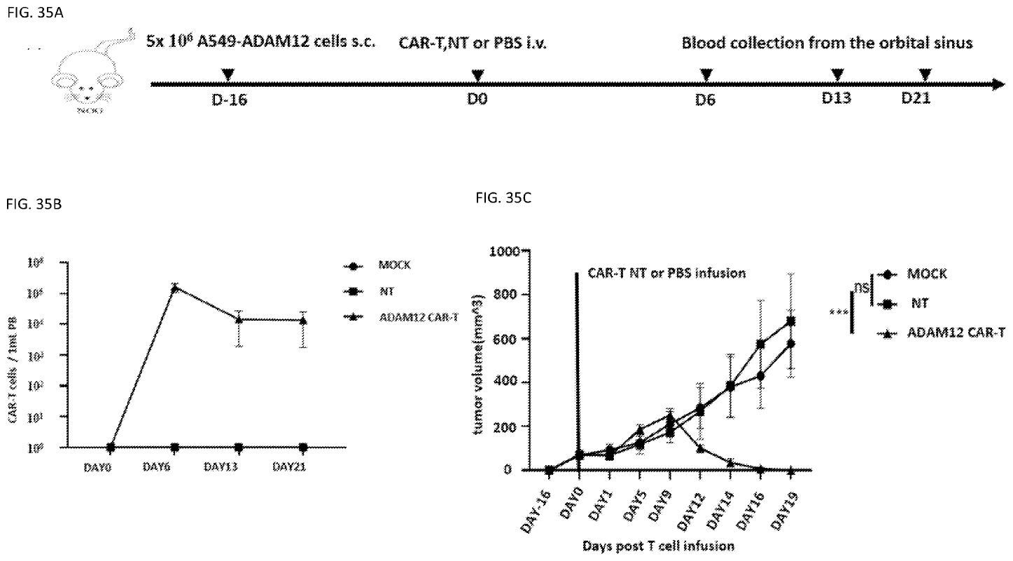

FIGS. 35A-35C: (FIG. 35A) Schematic illustration of the in vivo studies using ADAM12 CAR T cells. The tumor xenograft model was established by the subcutaneous injection of 5×10⁶ A549-ADAM12 cells into mice at D-16. Sixteen days later (D0), the inoculated mice were randomly divided into the MOCK group, NT group, and ADAM12 CAR T group and infused with 4.0×10⁶ non-transduced T cells or 4.0×10⁶ ADAM12 CAR T cells via their tail vein. The tumor volumes of the mice were measured every few days until Day 19. Peripheral blood was collected on D6, D13, and D21 for the analysis of T cell proliferation. (FIG. 35B) The proliferation of CAR T cells in the peripheral blood of mice in the ADAM12 CAR T group, NT group, and mock groups on D6, D13, and D21 was assessed by flow cytometry. (FIG. 35C) Tumor volumes of the mice over time. P value<0.05=*, <0.01=**, <0.001=***, <0.0001=****; P 0.05=no significance=NS.

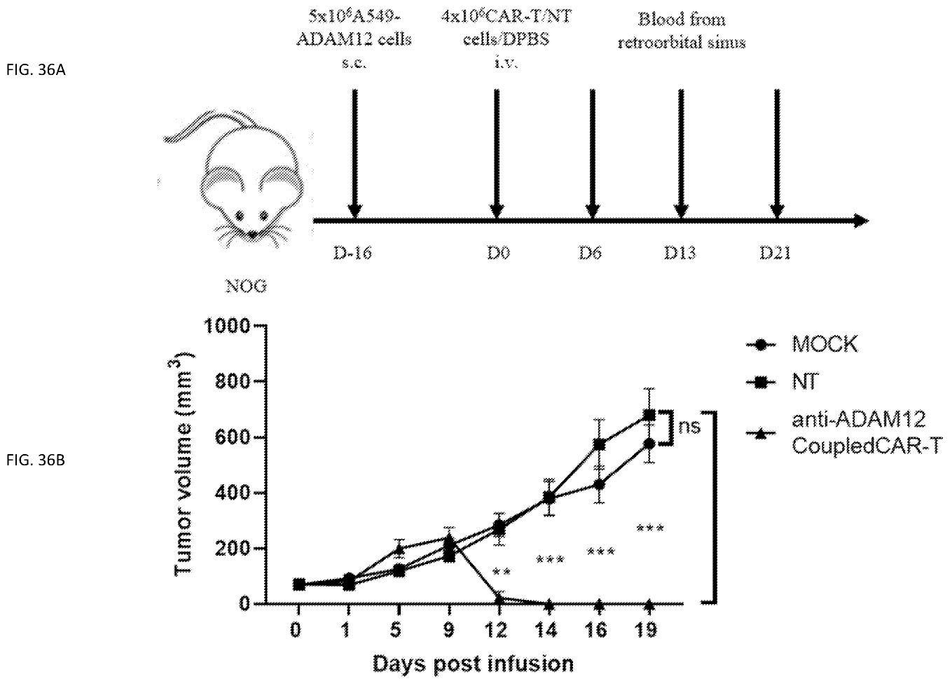

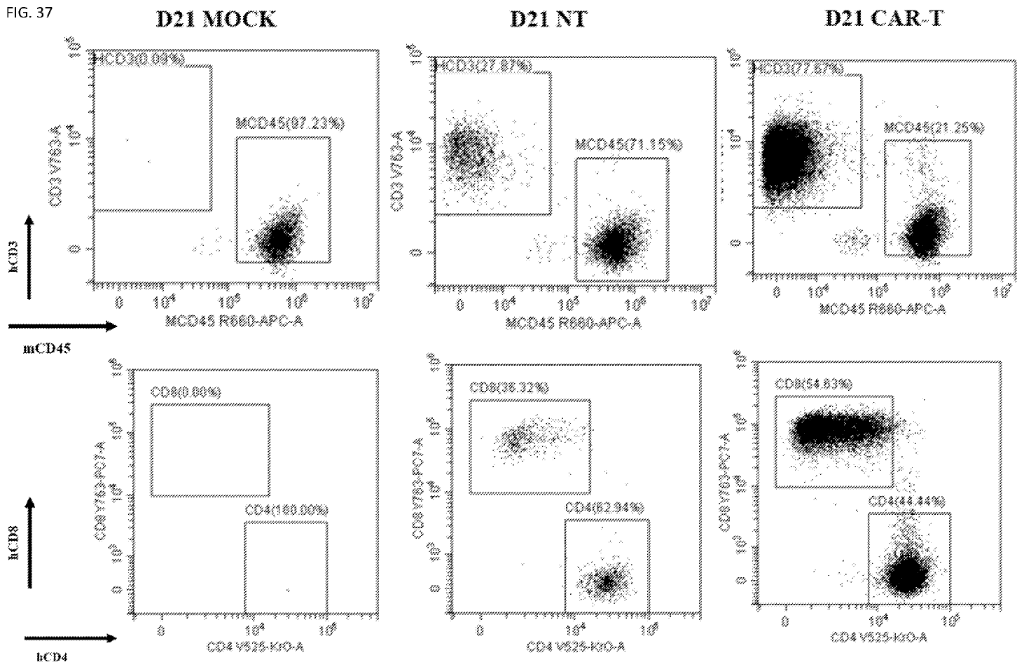

FIGS. 36A-36B shows mixed CAR T cells including ADAM12 CAR and CD19 CAR T (CoupledCAR®) cells exhibited good anti-tumor effect in mice. (FIG. 36A) Scheme for in vivo experiments: A subcutaneous tumor model was established by subcutaneous injection of 5.0×10⁶ A549-ADAM12 tumor cells into mice on D-16. After 16 days (D0), an equal volume of DPBS and 4.0×10⁶ CAR T cells were injected into the tail vein for treatment. Blood was taken from the marginal venous plexus on D6, D13 and D21 for flow cytometry analysis. (FIG. 36B) Changes in tumor volume of mice in each group over time, *P<0.05, **P<0.01, ***P<0.001, ns means no significant difference. FIG. 37 shows the ratio of CD4+ and CD8+ cells in the peripheral blood after blood was taken from the marginal venous plexus on D21.

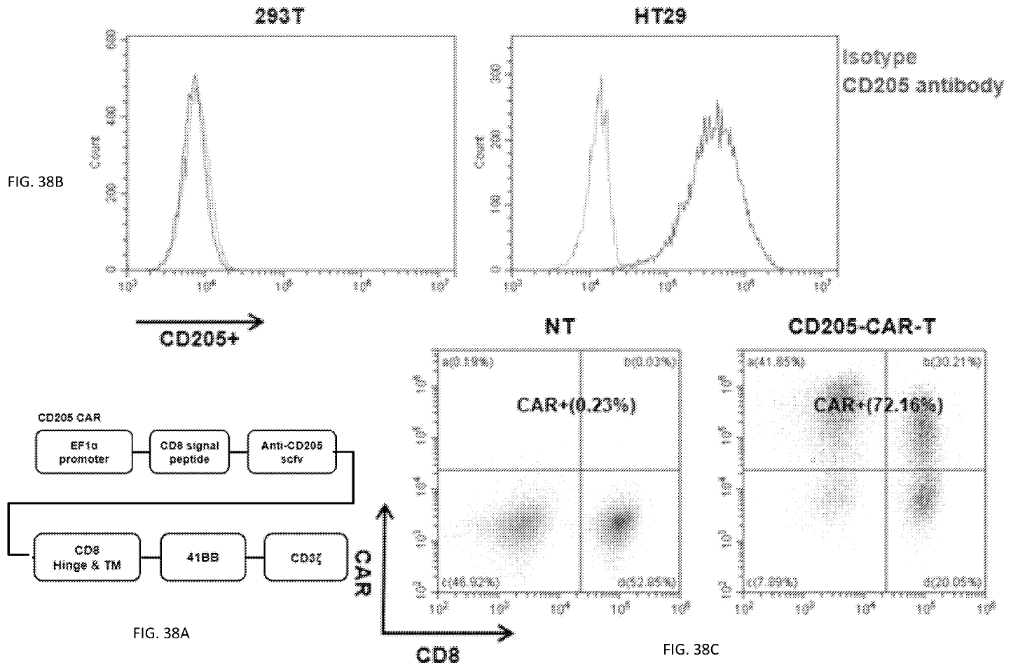

Further results of the in vitro experiments show that CAR T cells targeting CD205 (scFv: SEQ ID NO: 8; CAR: SEQ ID NO: 339) can be effectively activated, and when cultured with corresponding tumor cells, these CAR T cells can release a variety of cytokines to specifically kill tumor cells and proliferate. FIGS. 38A-38C shows CD205 CAR T exhibited good antitumor effect in vitro. (FIG. 38A) The figure shows the vector which includes CD205 scFv sequence, CD8 signal peptide, transmembrane region, co-stimulatory domain, and CD3ζ. (FIG. 38B) The expression of cell lines 293T and HT29 was detected by flow cytometry. (FIG. 38C) The expression of NT and CD205 CAR T was detected by flow cytometry.

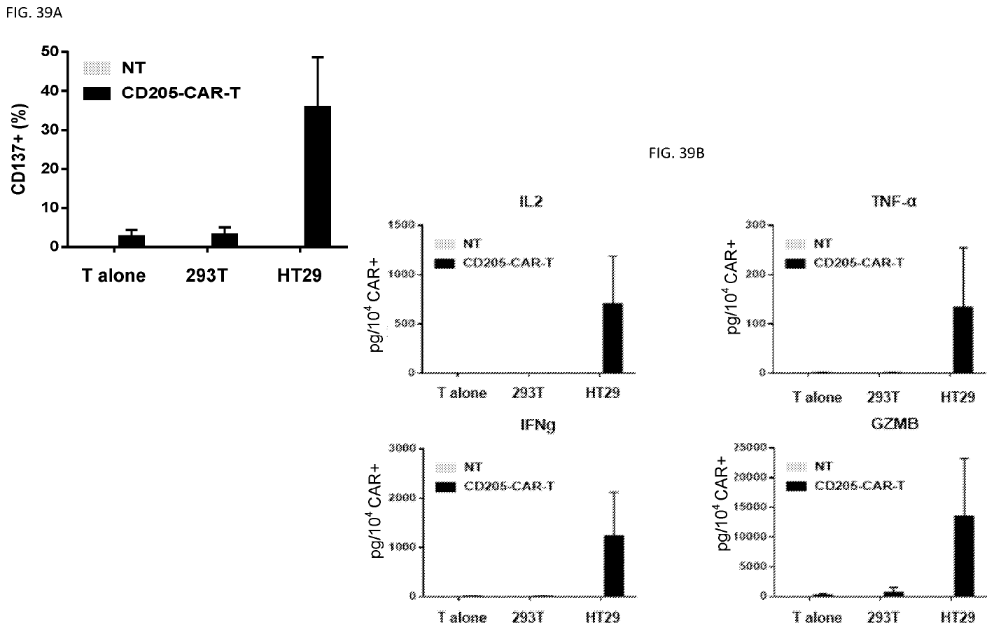

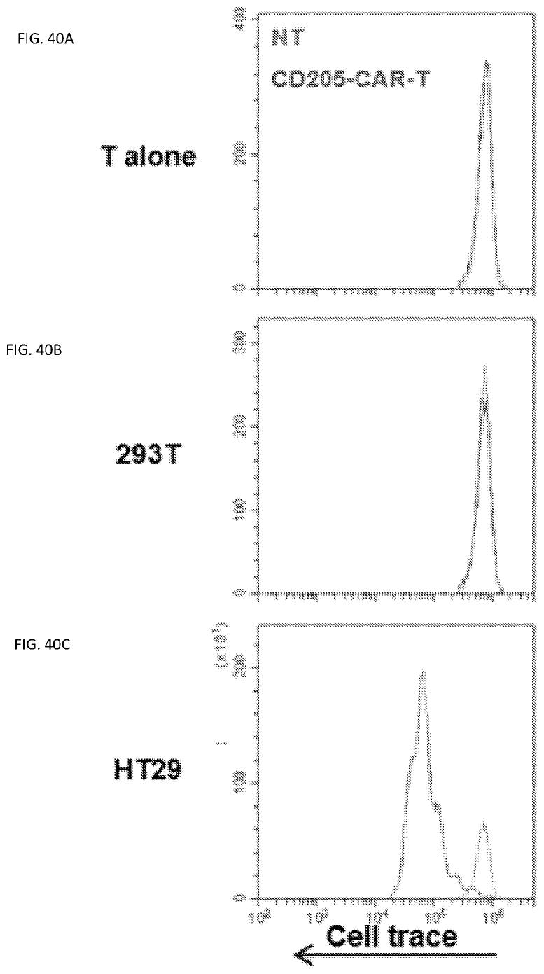

FIGS. 39A-39B show CD205 CAR T exhibited good antitumor effect in vitro. (FIG. 39A) After a variety of tumor cell lines were co-cultured with CAR T cells for 24 hours, the expression of T cell activation marker CD137 was detected by flow cytometry. (FIG. 39B) Cytometric Bear Array (CBA) was used to detect IL2, —TNF-α, IFN-γ, and Gramzyme B content in the supernatant of the cultures. The value is normalized and presented as pg per 10⁴ CAR T cells. FIGS. 40A-40C show the proliferation of each group of cells using CellTrace™ Cell Proliferation Kit, after CAR T cells and NT cells were co-cultured with the above positive tumor cells.

CD205 CAR T cells or control cells (non-transduced T cells) or PBS solution (Mock) were injected on D0 into NOG mice with established tumors from colon cancer cell line HT29-CD205 expressing CD205. Orbital sinus blood was then collected on D7, D14, and D21 post-injection to quantitate CAR T cells (by flow cytometry). Tumor volume was also measured on DO and at 2-4 day intervals for over 29 days. CD205 CAR T cells expanded in vivo up to 10⁶ cells/mL and were associated with limited tumor growth compared to controls. The results shows that CD205 CAR T cells can inhibit CD205-expressing HT29-CD205 tumor growth in vivo.

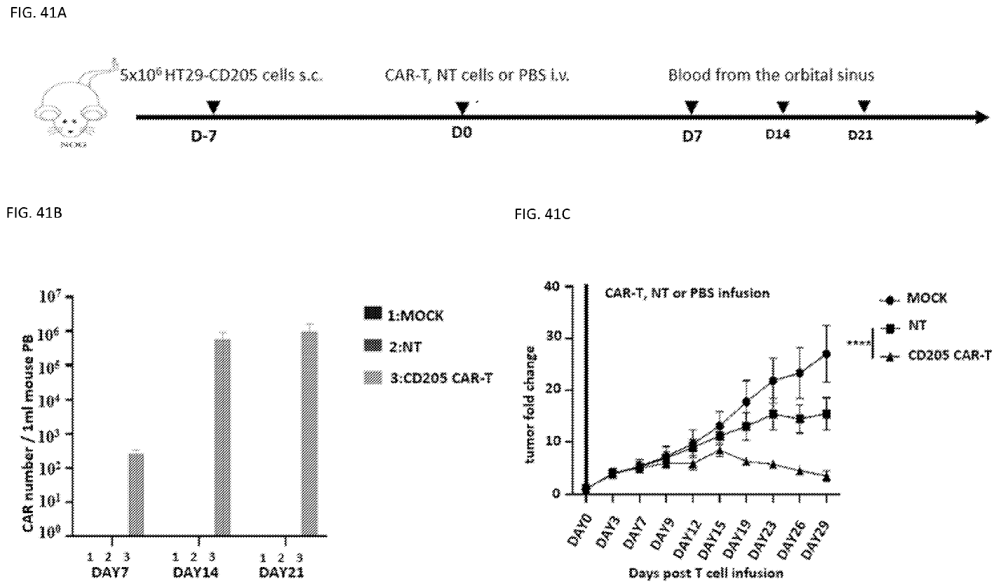

FIGS. 41A-41C: (FIG. 41A) Schematic illustration of the in vivo studies. The tumor xenograft model was established by the subcutaneous injection of 5.0×10⁶ HT29-CD205 cells (D-7). Seven days later (D0), the inoculated mice were randomly divided into the MOCK group and non-transduced T cells group and CAR T cell group, 1.0×10⁶ NT cells and 1.0×10⁶ CD205 CAR T cells via their tail vein. The tumor volumes of the mice were measured every several days until Day 29. Peripheral blood was collected on days 7, 14 and 21 for the analysis of T cell proliferation. (FIG. 41B) The proliferation of CAR T cells in the peripheral blood of mice in all of the CD205 CAR T, NT and MOCK groups on days 7, 14 and 21 was assessed by flow cytometry. (FIG. 41C) Tumor volumes change in the mice over time. P value<0.05=*, <0.01=**, <0.001=***, <0.0001=****; P 0.05=no significance=NS.

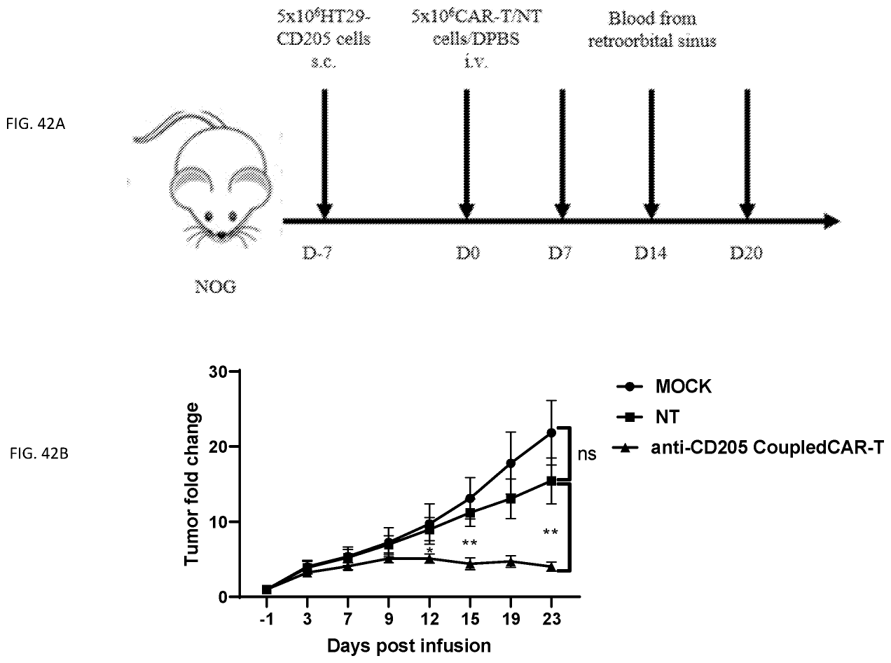

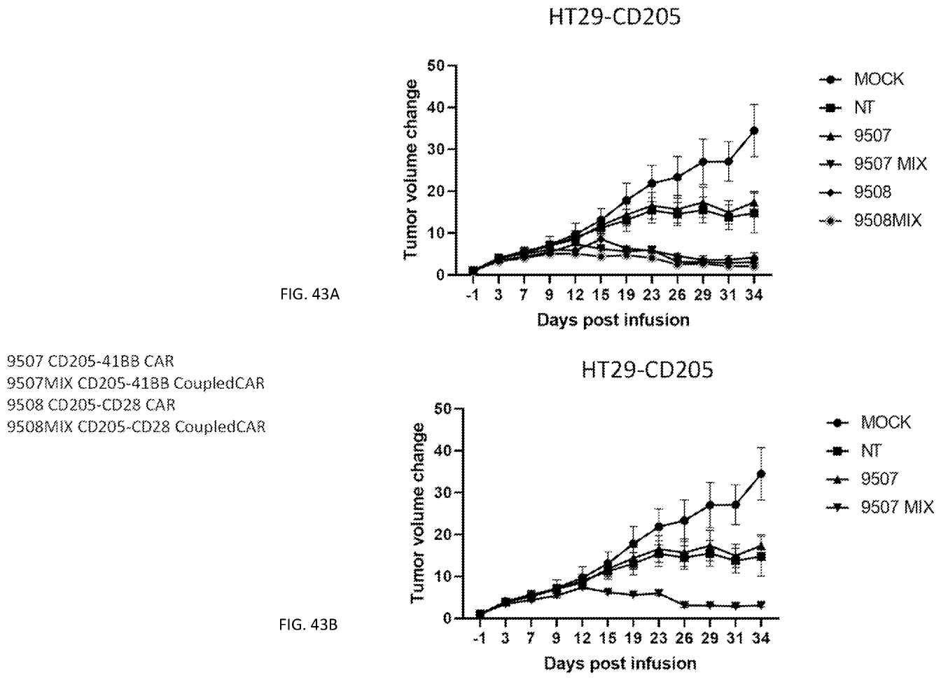

FIGS. 42A-42B show CD205 CAR T exhibited good anti-tumor effect in mice. (Blood was taken from the marginal venous plexus on D7, D14, and D20 days for flow cytometry analysis. Immunohistochemical staining (IHC) was used to detect the expression of CD205 in mouse subcutaneous tumors. On D20, blood from the marginal venous plexus was collected to detect the ratio of CD4+ and CD8+ cells in the peripheral blood. (FIG. 42B) Changes in tumor volume of mice in each group over time, *P<0.05, **P<0.01, ***P<0.001, ns means no significant difference. FIGS. 43A-43B show comparisons of anti-tumor activity of different infused cells. The experimental procedure was the same of that of FIGS. 42A-42B. As shown in FIGS. 43A-43B, CoupledCAR® T cells showed greater anti-tumor activity than single CAR T cells.

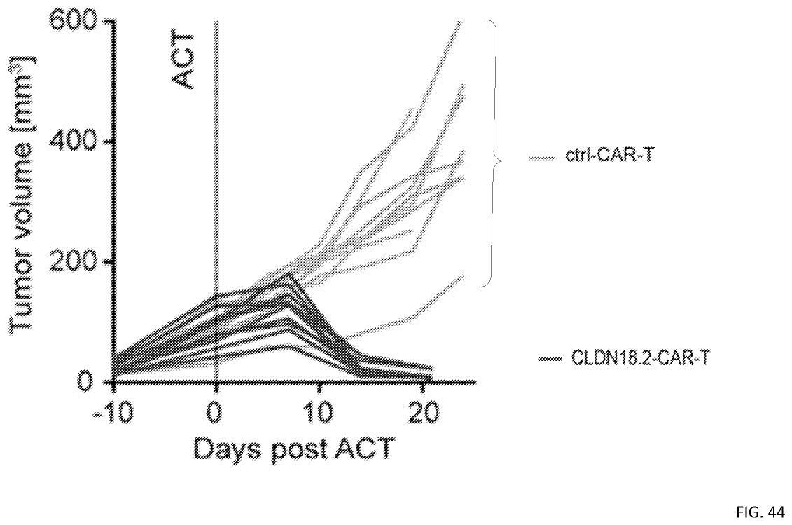

FIG. 44 shows CAR T cells targeting CLDN18.2 showed significant anti-tumor ability in mouse CLDN18.2 positive tumor model. The experiment procedure was similar to that described above and shown in FIG. 41. FIG. 44 shows that CAR T cells targeting CLDN18.2 (scFv: SEQ ID NO: 46; CAR: SEQ ID NO: 47) can significantly expand in tumor-bearing mice, and effectively kill tumors, which indicates strong anti-tumor activity. No obvious toxicity to the mice was observed.

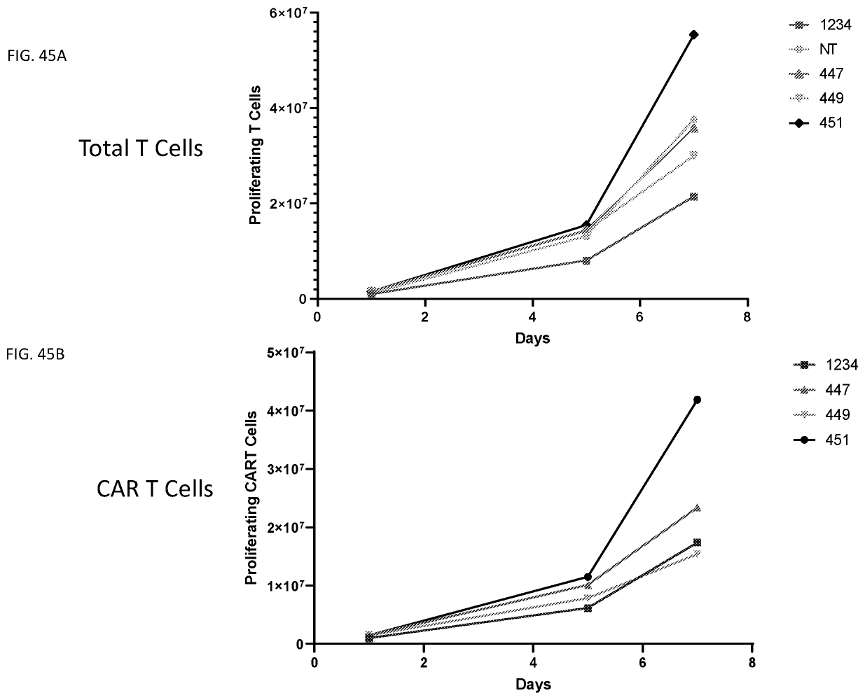

FIGS. 45A-45B show in vitro proliferation of ROR1 CAR T cells. Sequences and identifiers are provided in the table below. FIGS. 45A-45B show obvious advantages in culture proliferation in terms of the number of CAR T and the number of total T cells.

TABLE 5

| Cell Identifiers | Binding domain sequences | CAR Sequences |
|---|---|---|
| 447 | SEQ ID No: 34 | SEQ ID No: 35 |
| 449 | SEQ ID No: 256 | SEQ ID No: 257 |
| 451 | SEQ ID No: 259 | SEQ ID No: 260 |

### Example 9. Human In Vivo and In Vitro Data

Clinical studies were designed to assess the safety and efficacy of infusing autologous T cells modified to express several solid tumor markers specific for CAR/4-1BB/CD3-ζ into patients. Patients received CAR T cells directed to CD19 and a solid tumor antigen (MSLN or GPC3). T cells of the patients were obtained, modified, and infused into the patients. T cell responses of patients from the first and second arms were measured and compared using the following protocols, which were approved by the hospitals where the trials were conducted. All patients were provided with written informed consent. Information regarding these patients are provide below in Table 6 (SD: stable disease; PD: progressive disease; PR: partial remission; CR: complete remission; NR: no response).

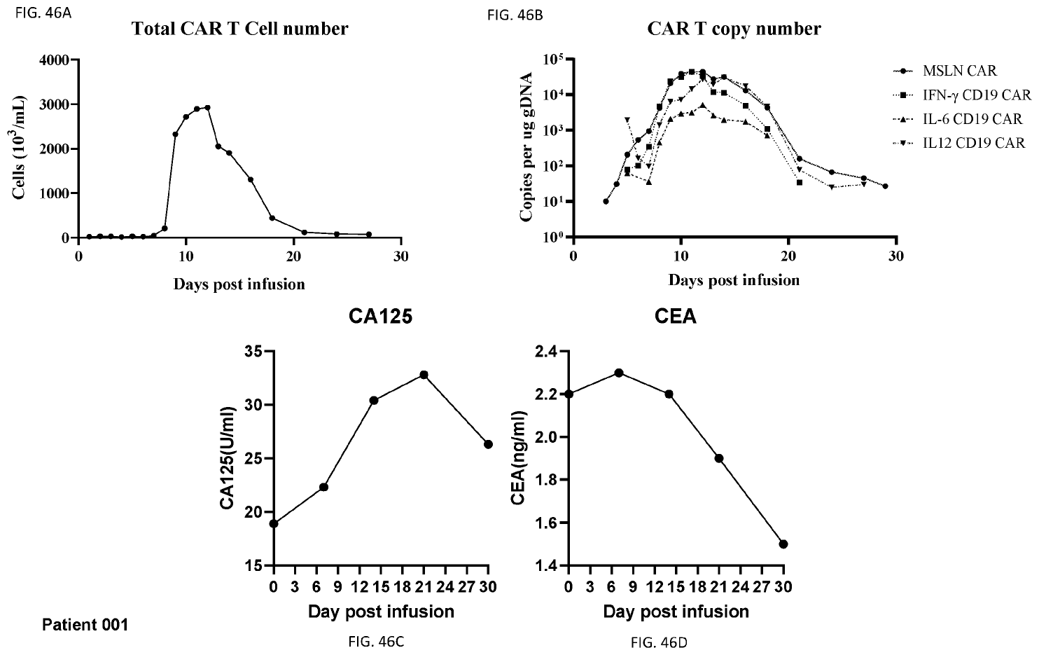

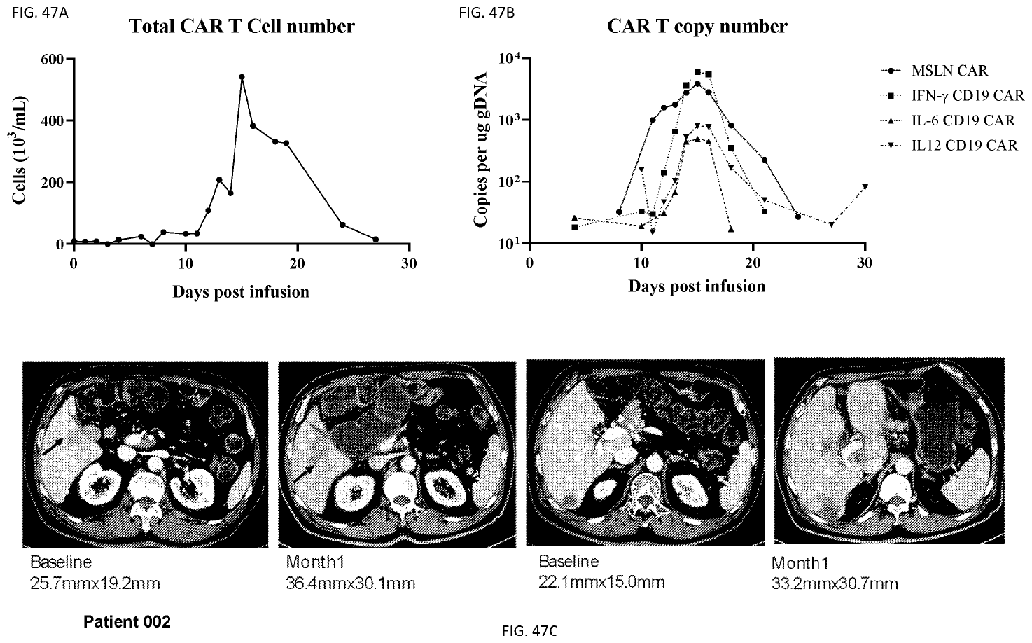

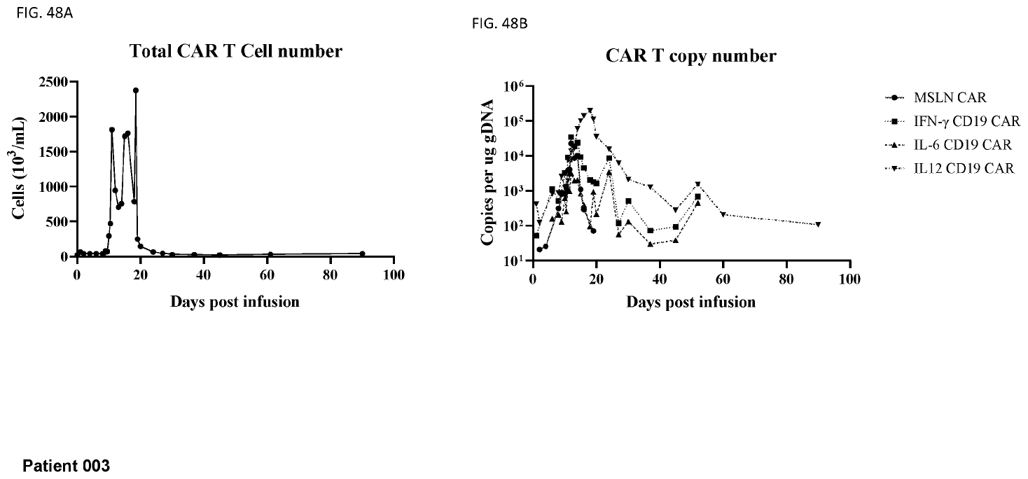

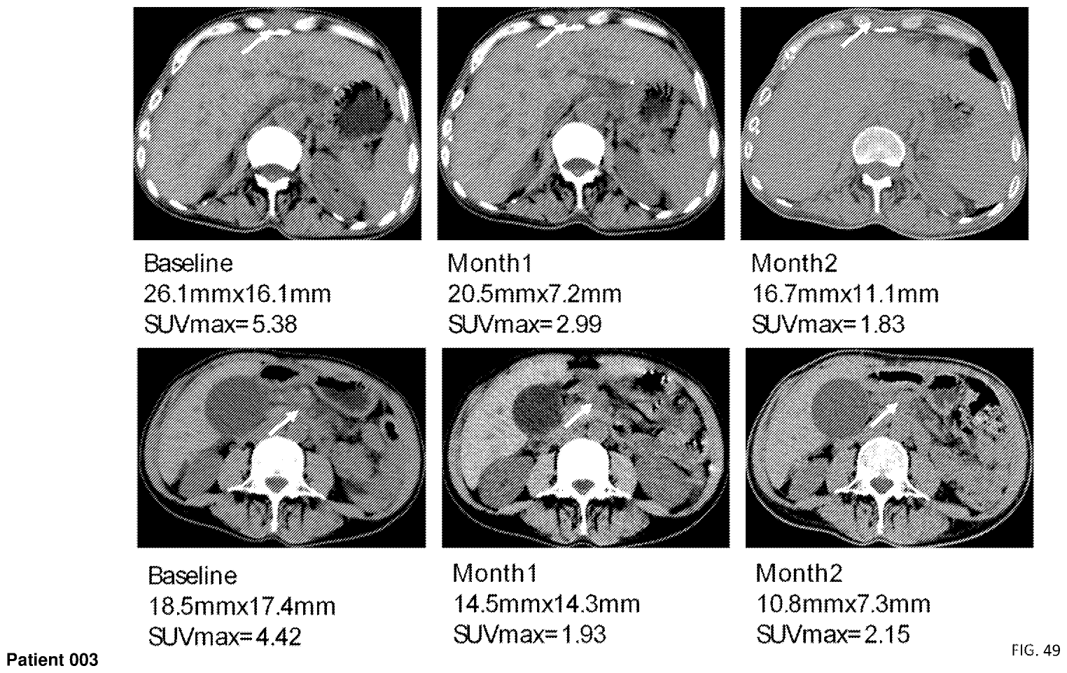

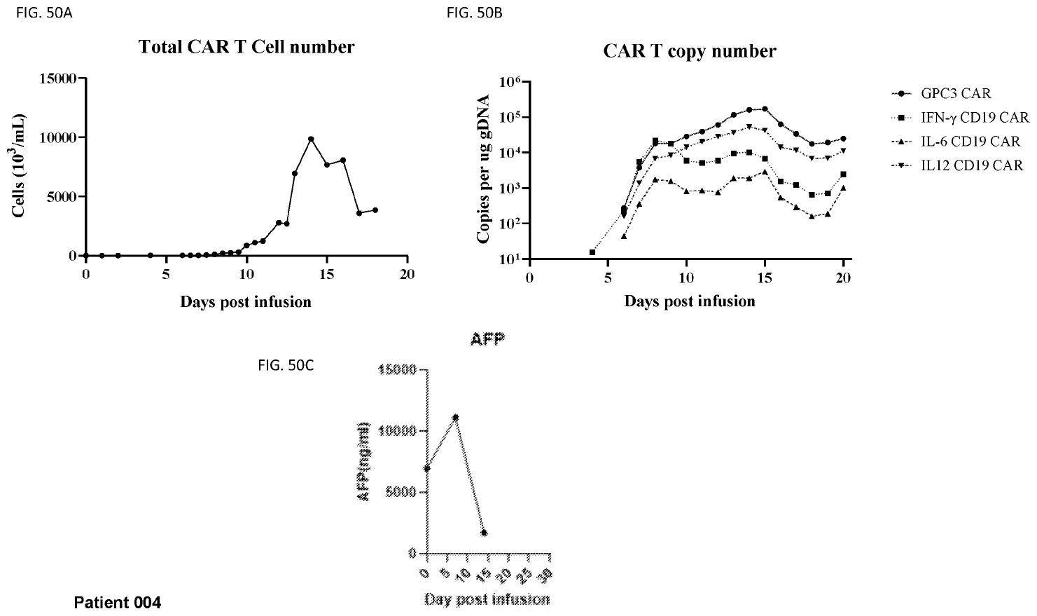

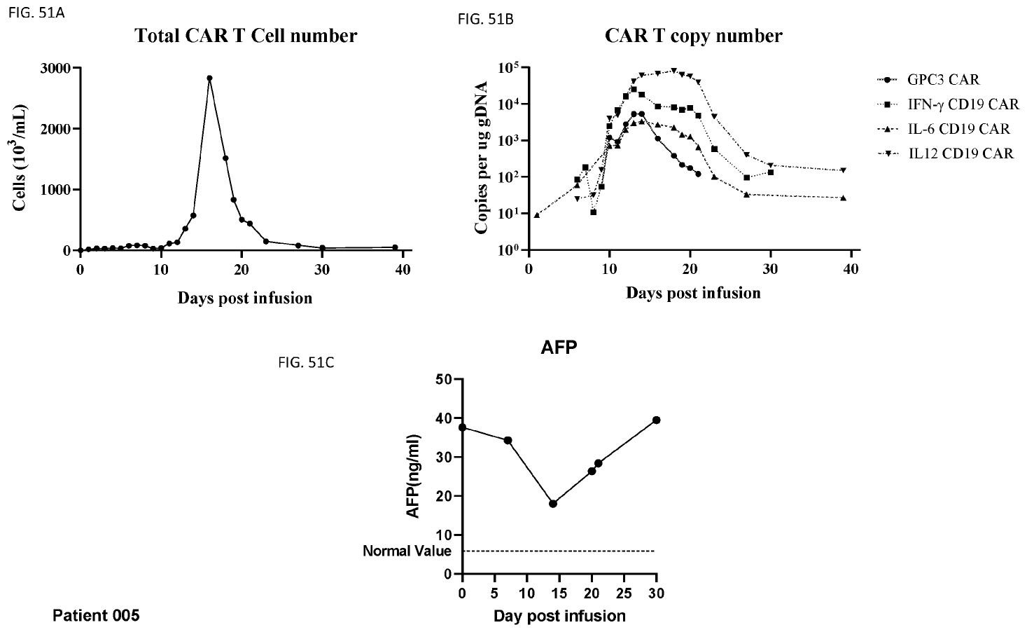

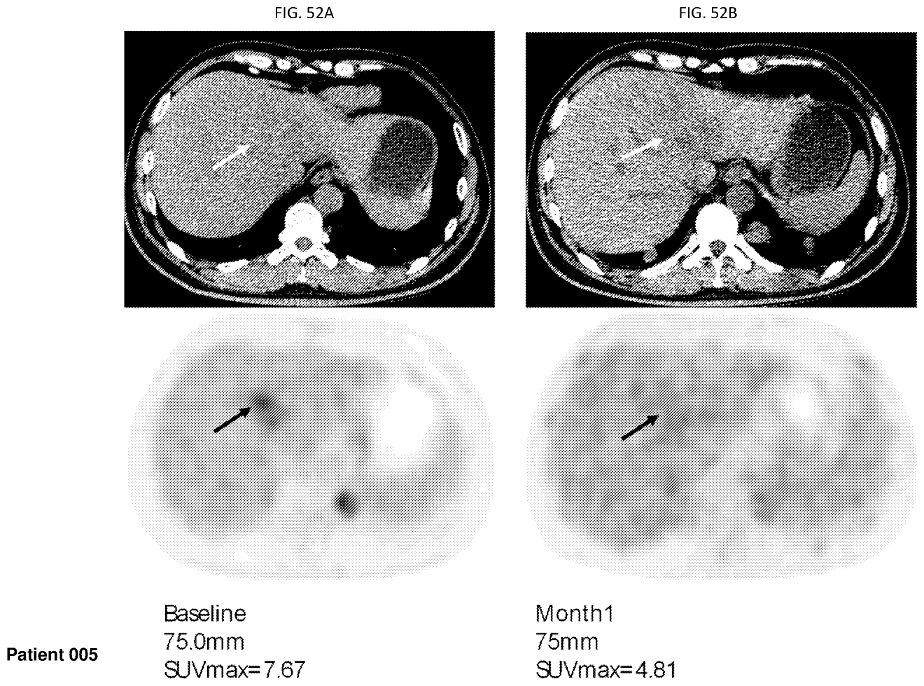

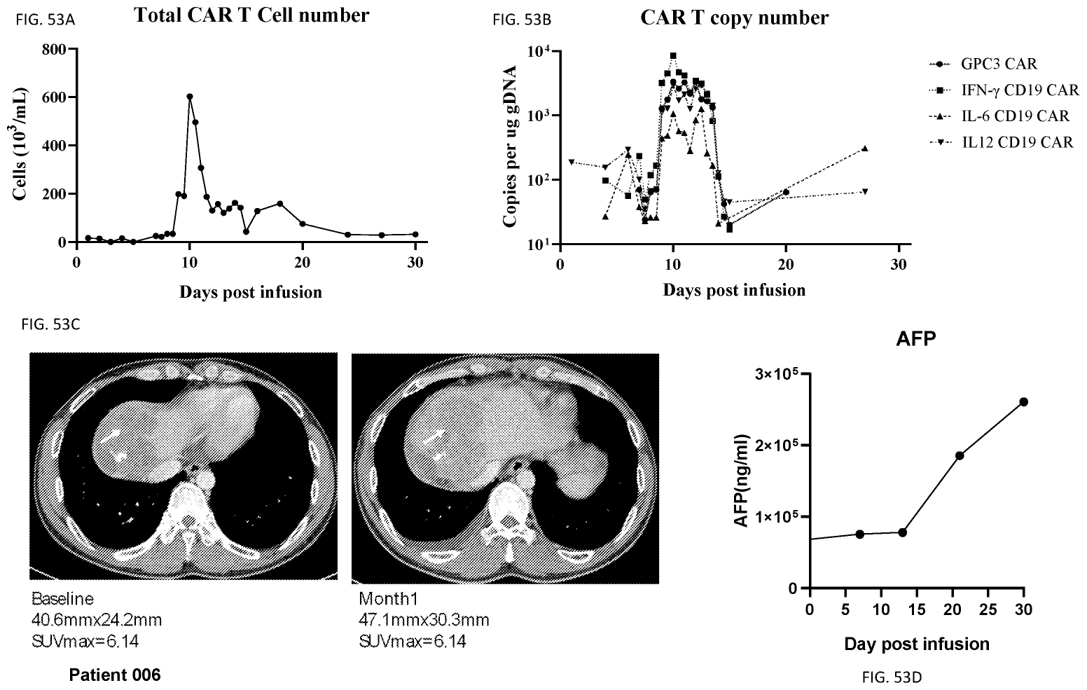

PBMCs were obtained from patients. Various lentiviral vectors encoding CAR molecules and/or cytokines were generated, transfected into the T cells, and further cultured for several days before performing the co-cultivation assay. More information can be found in Table 6 below. Techniques related to cell cultures, construction of cytotoxic T-lymphocyte assay can be found in “Control of large, established tumor xenografts with genetically retargeted human T cells containing CD28 and CD137 domains,” PNAS, Mar. 3, 2009, vol. 106 no. 9, 3360-3365, which is incorporated herein by reference in its entirety. T cells were mixed with vectors including a vector encoding CD19 CAR and IL-6, a vector encoding CD19 CAR and IL12, a vector encoding CD19 CAR and IFNγ, and a vector encoding a solid tumor CAR (MSLN or GPC3). Detailed methods can be found at PCT Publication No: WO2020146743, which is incorporated by its entirety. Patients' response to the infused cells are provided in FIGS. 46A-46D, 47A-47C, 48A-48B, 49, 50A-50C, 51A-51C, 52A-52B, and 53A-53D and Table 6 below. FIGS. 46A-46D show the proliferation of T cells and the reduction of tumor markers in Patient 001 after CAR T cell infusion. FIGS. 47A-47C show the proliferation of T cells and CT scanning results for Patient 002 after CAR T cell infusion. FIGS. 48A-48B show the proliferation of T cells in Patient 003 after CAR T cell infusion. FIG. 49 shows CT scanning results in Patient 003 after CAR T cell infusion. FIGS. 50A-50C show the proliferation of T cells and the reduction of a tumor marker in Patient 004 after CAR T cells infusion. FIGS. 51A-51C show the proliferation of T cells and the reduction of a tumor marker in Patient 005 after CAR T cell infusion. FIGS. 52A-52B show CT scanning results of Patient 005 after CAR T cells infusion. FIGS. 53A-53D show the proliferation of T cells, the reduction of a solid tumor marker, and CT scanning results of Patient 006 after CAR T cells infusion.

TABLE 6

| | | | | Binding | | |
|---|---|---|---|---|---|---|
| | | | Dose | Domain scFv | CAR | |
| Patient | Cancer | Target | (CART/KG) | sequences | sequences | Response |
| 001 | Bile Duct | MSLN | 1.09 × 10⁶ | SEQ ID NO: | SEQ ID NO: | M1: Tumor |
| | Cancer | | | 26 | 27 | markers |
| | | | | | | such as |
| | | | | | | CA125 and |
| | | | | | | CEA |
| | | | | | | decreased |
| | | | | | | at one |
| | | | | | | month post |
| | | | | | | infusion |
| 002 | Bile Duct | MSLN | 2.84 × 10⁵ | SEQ ID NO: | SEQ ID NO: | M1: PD |
| | Cancer | | | 26 | 27 | |
| 003 | Pancreatic | MSLN | 1.17 × 10⁵ | SEQ ID NO: | SEQ ID NO: | M1: Target |
| | Cancer | | | 26 | 27 | lesion |
| | | | | | | Shrinkage: −22% |
| 004 | Liver | GPC3 | 2.83 × 10⁵ | SEQ ID NO: | SEQ ID NO: | M1: Tumor |
| | Cancer | | | 16 | 17 | markers |
| | | | | | | such as |
| | | | | | | AFP |
| | | | | | | decreased |
| | | | | | | at one |
| | | | | | | month post |
| | | | | | | infusion |
| 005 | Liver | GPC3 | 3.27 × 10⁵ | SEQ ID NO: | SEQ ID NO: | M1: Partial |
| | Cancer | | | 16 | 17 | Metabolic |
| | | | | | | Response |
| | | | | | | (Percist1.0) |
| | | | | | | Target |
| | | | | | | lesion |
| | | | | | | Shrinkage: −32% |
| 006 | Liver | GPC3 | 1.14 × 10⁶ | SEQ ID NO: | SEQ ID NO: | M1: SD |
| | Cancer | | | 16 | 16 | |

TABLE 7

| | SEQ | | SEQ | | SEQ | | SEQ |
|---|---|---|---|---|---|---|---|
| ID | ID | ID | ID | ID | ID | ID | NO |
| CD70 antigen | 1 | CD70 binding | 85 | GPC3 | 169 | MUC17 Binding | 253 |
| | | domain 2 | | binding | | domain 4 | |
| | | | | domain 9 | | | |
| CD70 binding | 2 | CD70 CAR 3 | 86 | GPC3 CAR | 170 | MUC17 CAR 7 | 254 |
| domain | | | | 17 | | | |
| CD70 CAR | 3 | CD70 CAR 4 | 87 | GPC3 CAR | 171 | MUC17 CAR 8 | 255 |
| | | | | 18 | | | |
| CD205 | 4 | CD70 binding | 88 | GPC3 | 172 | ROR1 binding | 256 |
| antigen | | domain 3 | | binding | | domain 2 | |
| | | | | domain 10 | | | |
| CD205 | 5 | CD70 CAR 5 | 89 | GPC3 CAR | 173 | ROR1 CAR 3 | 257 |
| binding | | | | 19 | | | |
| domain 1 | | | | | | | |
| CD205 CAR | 6 | CD70 CAR 6 | 90 | GPC3 CAR | 174 | ROR1 CAR 4 | 258 |
| 1 | | | | 20 | | | |
| CD205 CAR | 7 | CD70 binding | 91 | GPC3 | 175 | ROR1 binding | 259 |
| 2 | | domain 4 | | binding | | domain3 | |
| | | | | domain 11 | | | |
| CD205 | 8 | CD70 CAR 7 | 92 | GPC3 CAR | 176 | ROR1 CAR 5 | 260 |
| binding | | | | 21 | | | |
| domain 2 | | | | | | | |
| CD205 CAR | 9 | CD70 CAR 8 | 93 | GPC3 CAR | 177 | ROR1 CAR 6 | 261 |
| 3 | | | | 22 | | | |
| CD205 CAR | 10 | CD70 binding | 94 | HAVCR1 | 178 | ROR1 binding | 262 |
| 4 | | domain 5 | | antigen | | domain 4 | |
| CLDN6 | 11 | CD70 CAR 9 | 95 | HAVCR1 | 179 | ROR1 CAR 7 | 263 |
| antigen | | | | binding | | | |
| | | | | domain | | | |
| CLDN6 | 12 | CD70 CAR 10 | 96 | HAVCR1 | 180 | ROR1 CAR 8 | 264 |
| binding | | | | CAR 1 | | | |
| domain | | | | | | | |
| CLDN6 | 13 | CD70 binding | 97 | HAVCR1 | 181 | ROR1 binding | 265 |
| binding | | domain 6 | | CAR 2 | | domain5 | |
| domain 2 | | | | | | | |
| CLDN6 CAR | 14 | CD70 CAR 11 | 98 | HAVCR1 | 182 | ROR1 CAR9 | 266 |
| 1 | | | | binding | | | |
| | | | | domain 2 | | | |
| GPC3 | 15 | CD70 CAR 12 | 99 | HAVCR1 | 183 | ROR1 CAR 10 | 267 |
| antigen | | | | CAR 3 | | | |
| GPC3 | 16 | CD70 binding | 100 | HAVCR1 | 184 | ROR1 binding | 268 |
| binding | | domain 7 | | CAR 4 | | domain6 | |
| domain 1 | | | | | | | |
| GPC3 CAR 1 | 17 | CD70 CAR 13 | 101 | HAVCR1 | 185 | ROR1 CAR 11 | 269 |
| | | | | binding | | | |
| | | | | domain 3 | | | |
| GPC3 CAR 2 | 18 | CD70 CAR 14 | 102 | HAVCR1 | 186 | ROR1 CAR 12 | 270 |
| | | | | CAR 5 | | | |
| GPC3 | 19 | CD70 binding | 103 | HAVCR1 | 187 | CD70 binding | 271 |
| binding | | domain 8 | | CAR 6 | | domain 10 | |
| domain 2 | | | | | | | |
| GPC3 CAR 3 | 20 | CD70 CAR 15 | 104 | LRRC15 | 188 | CD70 CAR 19 | 272 |
| | | | | antigen | | | |
| GPC3 CAR 4 | 21 | CD70 CAR 16 | 105 | LRRC15 | 189 | CD70 CAR 20 | 273 |
| | | | | binding | | | |
| | | | | domain 1 | | | |
| MSLN | 22 | CD70 binding | 106 | LRRC15 | 190 | CD70 binding | 274 |
| antigen | | domain 9 | | CAR 1 | | domain 11 | |
| MSLN | 23 | CD70 CAR 17 | 107 | LRRC15 | 191 | CD70 CAR 21 | 275 |
| binding | | | | CAR 2 | | | |
| domain 1 | | | | | | | |
| MSLN CAR 1 | 24 | CD70 CAR 18 | 108 | LRRC15 | 192 | CD70 CAR 22 | 276 |
| | | | | binding | | | |
| | | | | domain 2 | | | |
| MSLN CAR 2 | 25 | CD205 | 109 | LRRC15 | 193 | CD70 binding | 277 |
| | | antigen 3 | | CAR 3 | | domain 12 | |
| MSLN | 26 | CD205 CAR 5 | 110 | LRRC15 | 194 | CD70 CAR 23 | 278 |
| binding | | | | CAR 4 | | | |
| domain 2 | | | | | | | |
| MSLN CAR 3 | 27 | CD205 CAR 6 | 111 | LRRC15 | 195 | CD70 CAR 24 | 279 |
| | | | | binding | | | |
| | | | | domain 3 | | | |
| MSLN CAR 4 | 28 | CD205 | 112 | LRRC15 | 196 | CD70 binding | 280 |
| | | antigen 3 | | CAR 5 | | domain 13 | |
| MUC16 | 29 | CD205 CAR 5 | 113 | LRRC15 | 197 | CD70 CAR 25 | 281 |
| antigen | | | | CAR 6 | | | |
| MUC16 | 30 | CD205 CAR 6 | 114 | MSLN | 198 | CD70 CAR 26 | 282 |
| binding | | | | binding | | | |
| domain | | | | domain 3 | | | |
| MUC16 CAR | 31 | CLDN6 CAR 2 | 115 | MSLN CAR | 199 | CD70 binding | 283 |
| 1 | | | | 5 | | domain 14 | |
| MUC16 CAR | 32 | CLDN6 CAR 3 | 116 | MSLN CAR | 200 | CD70 CAR 27 | 284 |
| 2 | | | | 6 | | | |
| ROR1 | 33 | CLDN6 CAR 4 | 117 | MSLN | 201 | CD70 CAR 28 | 285 |
| antigen | | | | binding | | | |
| | | | | domain 4 | | | |
| ROR1 | 34 | DLL3 antigen | 118 | MSLN CAR | 202 | CD70 binding | 286 |
| binding | | | | 7 | | domain 15 | |
| domain | | | | | | | |
| ROR1 CAR 1 | 35 | DLL3 binding | 119 | MSLN CAR | 203 | CD70 CAR 29 | 287 |
| | | domain | | 8 | | | |
| ROR1 CAR 2 | 36 | DLL3 CAR 1 | 120 | MSLN | 204 | CD70 CAR 30 | 288 |
| | | | | binding | | | |
| | | | | domain 5 | | | |
| ADAM12 | 37 | DLL3 CAR 2 | 121 | MSLN CAR | 205 | CD70 binding | 289 |
| antigen | | | | 9 | | domain 16 | |
| ADAM12 | 38 | DLL3 binding | 122 | MSLN CAR | 206 | CD70 CAR 31 | 290 |
| binding | | domain 2 | | 10 | | | |
| domain | | | | | | | |
| ADAM12 | 39 | DLL3 CAR 3 | 123 | MSLN | 207 | CD70 CAR 32 | 291 |
| CAR 1 | | | | binding | | | |
| | | | | domain 6 | | | |
| ADAM12 | 40 | DLL3 CAR 4 | 124 | MSLN CAR | 208 | CD70 binding | 292 |
| CAR 2 | | | | 11 | | domain 17 | |
| ALPP antigen | 41 | DLL3 binding | 125 | MSLN CAR | 209 | CD70 CAR 33 | 293 |
| | | domain 3 | | 12 | | | |
| ALPP binding | 42 | DLL3 CAR 5 | 126 | MSLN | 210 | CD70 CAR 34 | 294 |
| domain | | | | binding | | | |
| | | | | domain 7 | | | |
| ALPP CAR 1 | 43 | DLL3 CAR 6 | 127 | MSLN CAR | 211 | CD70 binding | 295 |
| | | | | 13 | | domain 18 | |
| ALPP CAR 1 | 44 | DLL3 binding | 128 | MSLN CAR | 212 | CD70 CAR 35 | 296 |
| | | domain 4 | | 14 | | | |
| CLDN18.2 | 45 | DLL3 CAR 7 | 129 | MSLN | 213 | CD70 CAR 36 | 297 |
| antigen | | | | binding | | | |
| | | | | domain 8 | | | |
| CLDN18.2 | 46 | DLL3 CAR 8 | 130 | MSLN CAR | 214 | CLDN18.2 | 298 |
| binding | | | | 15 | | binding domain | |
| domain | | | | | | 2 | |
| CLDN18.2 | 47 | DLL3 binding | 131 | MSLN CAR | 215 | CLDN18.2 CAR | 299 |
| CAR 1 | | domain 5 | | 16 | | 3 | |
| CLDN18.2 | 48 | DLL3 CAR 9 | 132 | MSLN | 216 | CLDN18.2 CAR | 300 |
| CAR 2 | | | | binding | | 4 | |
| | | | | domain 9 | | | |
| ACPP | 49 | DLL3 CAR 10 | 133 | MSLN CAR | 217 | CLDN18.2 | 301 |
| binding | | | | 17 | | binding domain | |
| domain | | | | | | 3 | |
| ACPP CAR 1 | 50 | DLL3 binding | 134 | MSLN CAR | 218 | CLDN18.2 CAR | 302 |
| | | domain 6 | | 18 | | 5 | |
| ACPP CAR 2 | 51 | DLL3 CAR 11 | 135 | MSLN | 219 | CLDN18.2 CAR | 303 |
| | | | | binding | | 6 | |
| | | | | domain 10 | | | |
| ADAM12 | 52 | DLL3 CAR 12 | 136 | MSLN CAR | 220 | CLDN18.2 | 304 |
| binding | | | | 19 | | binding domain | |
| domain 2 | | | | | | 4 | |
| ADAM12 | 53 | FAP Antigen | 137 | MSLN CAR | 221 | CLDN18.2 CAR | 305 |
| CAR 3 | | | | 20 | | 7 | |
| ADAM12 | 54 | FAP binding | 138 | MSLN | 222 | CLDN18.2 CAR | 306 |
| CAR 4 | | domain 1 | | binding | | 8 | |
| | | | | domain11 | | | |
| ADAM12 | 55 | FAP CAR 1 | 139 | MSLN CAR | 223 | CLDN18.2 | 307 |
| binding | | | | 21 | | binding domain | |
| domain 3 | | | | | | 5 | |
| ADAM12 | 56 | FAP CAR 2 | 140 | MSLN CAR | 224 | CLDN18.2 CAR | 308 |
| CAR 5 | | | | 22 | | 9 | |
| ADAM12 | 57 | FAP binding | 141 | MSLN | 225 | CLDN18.2 CAR | 309 |
| CAR 6 | | domain 2 | | binding | | 10 | |
| | | | | domain 12 | | | |
| AFP antigen | 58 | FAP CAR 3 | 142 | MSLN CAR | 226 | CLDN18.2 | 310 |
| | | | | 23 | | binding domain6 | |
| AFP binding | 59 | FAP CAR 4 | 143 | MSLN CAR | 227 | CLDN18.2 CAR | 311 |
| domain | | | | 24 | | 11 | |
| AFP CAR 1 | 60 | GPC2 antigen | 144 | MSLN | 228 | CLDN18.2 CAR | 312 |
| | | | | binding | | 12 | |
| | | | | domain 13 | | | |
| AFP CAR 2 | 61 | GPC2 binding | 145 | MSLN CAR | 229 | CLDN18.2 | 313 |
| | | domain | | 25 | | binding domain7 | |
| AFP binding | 62 | GPC2 CAR 1 | 146 | MSLN CAR | 230 | CLDN18.2 CAR | 314 |
| domain 2 | | | | 26 | | 13 | |
| AFP CAR 3 | 63 | GPC2 CAR 2 | 147 | MUC16 | 231 | CLDN18.2 CAR | 315 |
| | | | | Binding | | 14 | |
| | | | | domain 2 | | | |
| AFP CAR 4 | 64 | GPC3 binding | 148 | MUC16 | 232 | CLDN18.2 | 316 |
| | | domain 2 | | CAR 3 | | binding domain8 | |
| AFP binding | 65 | GPC3 CAR 3 | 149 | MUC16 | 233 | CLDN18.2 CAR | 317 |
| domain 3 | | | | CAR 4 | | 15 | |
| AFP CAR 5 | 66 | GPC3 CAR 4 | 150 | MUC16 | 234 | CLDN18.2 CAR | 318 |
| | | | | Binding | | 16 | |
| | | | | domain 3 | | | |
| AFP CAR 6 | 67 | GPC3 binding | 151 | MUC16 | 235 | FAP binding | 319 |
| | | domain 3 | | CAR 5 | | domain 3 | |
| ALPP binding | 68 | GPC3 CAR 5 | 152 | MUC16 | 236 | FAP CAR 5 | 320 |
| domain 2 | | | | CAR 6 | | | |
| ALPP CAR 3 | 69 | GPC3 CAR 6 | 153 | MUC16 | 237 | FAP CAR 6 | 321 |
| | | | | Binding | | | |
| | | | | domain 4 | | | |
| ALPP CAR 4 | 70 | GPC3 binding | 154 | MUC16 | 238 | FAP binding | 322 |
| | | domain 4 | | CAR 7 | | domain 4 | |
| ALPP binding | 71 | GPC3 CAR 7 | 155 | MUC16 | 239 | FAP CAR 5 | 323 |
| domain 3 | | | | CAR 8 | | | |
| ALPP CAR 5 | 72 | GPC3 CAR 8 | 156 | MUC16 | 240 | FAP CAR 6 | 324 |
| | | | | Binding | | | |
| | | | | domain 5 | | | |
| ALPP CAR 6 | 73 | GPC3 binding | 157 | MUC16 | 241 | FAP binding | 325 |
| | | domain 5 | | CAR 9 | | domain 5 | |
| ALPP binding | 74 | GPC3 CAR 9 | 158 | MUC16 | 242 | FAP CAR 5 | 326 |
| domain 4 | | | | CAR 10 | | | |
| ALPP CAR 7 | 75 | GPC3 CAR 10 | 159 | MUC17 | 243 | FAP CAR 6 | 327 |
| | | | | Antigen | | | |
| ALPP CAR 8 | 76 | GPC3 binding | 160 | MUC17 | 244 | FAP binding | 328 |
| | | domain 6 | | Binding | | domain 6 | |
| | | | | domain 1 | | | |
| ALPP binding | 77 | GPC3 CAR 11 | 161 | MUC17 | 245 | FAP CAR 5 | 329 |
| domain 5 | | | | CAR 1 | | | |
| ALPP CAR 9 | 78 | GPC3 CAR 12 | 162 | MUC17 | 246 | FAP CAR 6 | 330 |
| | | | | CAR 2 | | | |
| ALPP CAR | 79 | GPC3 binding | 163 | MUC17 | 247 | FAP binding | 331 |
| 10 | | domain 7 | | Binding | | domain 7 | |
| | | | | domain 2 | | | |
| ALPP binding | 80 | GPC3 CAR 13 | 164 | MUC17 | 248 | FAP CAR 5 | 332 |
| domain 6 | | | | CAR 3 | | | |
| ALPP CAR | 81 | GPC3 CAR 14 | 165 | MUC17 | 249 | FAP CAR 6 | 333 |
| 11 | | | | CAR 4 | | | |
| ALPP CAR | 82 | GPC3 binding | 166 | MUC17 | 250 | FAP binding | 334 |
| 12 | | domain 8 | | Binding | | domain 8 | |
| | | | | domain 3 | | | |
| ALPP CAR | 83 | GPC3 CAR 15 | 167 | MUC17 | 251 | FAP CAR 5 | 335 |
| 13 | | | | CAR 5 | | | |
| ALPP CAR | 84 | GPC3 CAR 16 | 168 | MUC17 | 252 | FAP CAR 6 | 336 |
| 14 | | | | CAR 6 | | | |
| | | | | | | FCRL1 antigen | 337 |
| | | | | | | CD205 CAR 9 | 339 |

Related sequences are provided in this Application and Innovative Cellular Therapeutics' PCT Patent Applications Nos: PCT/CN2016/075061, PCT/CN2018/08891, and PCT/US19/13068, which are incorporated by reference in their entirety.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. An isolated nucleic acid encoding a chimeric antigen receptor (CAR), wherein the CAR comprises an extracellular domain, a transmembrane domain, and an intracellular domain, wherein the extracellular domain binds an antigen of a solid tumor and wherein the antigen comprises FCR1, MSLN, GPC-3, ALPP, CD70, CLDN6, ROR1, CD205, ACPP, ADAM12, or CLDN18.2, wherein the CAR comprises an amino acid sequence selected from the group consisting of SEQ ID NOs: 34, 35, 256, 257, 259, 260, 42, 43, 23, 24, 2, 3, 49, 50, 14, 12, 16, 17, 55, 56, 8, 339, 46, and 47.

2-13. (canceled)

14. The isolated nucleic acid of claim 1, wherein the intracellular domain comprises a co-stimulatory signaling region comprising an intracellular signaling domain of CD27, CD28, 4-1BB, OX40, CD30, CD40, PD-1, ICOS, lymphocyte function-associated antigen-1 (LFA-1), CD2, CD7, LIGHT, NKG2C, B7-H3, or any combination thereof.

15. The isolated nucleic acid of claim 1, wherein the intracellular domain comprises a CD3 zeta signaling domain.

16. A vector comprising the isolated nucleic acid of claim 1.

17. A CAR encoded by the isolated nucleic acid or vector of claim 1.

18. A modified cell comprising the isolated nucleic acid of claim 1, wherein the modified cell comprises a T cell.

19. The modified cell of claim 30, wherein the modified cell comprises a dominant negative form of a receptor associated with an immune checkpoint inhibitor, and optionally wherein the immune checkpoint inhibitor is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRD, natural killer cell receptor 2B4 (2B4), and CD 160.

20. The modified cell of claim 19, wherein the immune checkpoint inhibitor is modified PD-1, and optionally wherein the modified PD-1 lacks a functional PD-1 intracellular domain for PD-1 signal transduction; interferes with a pathway between PD-1 of a human T cell of the human cells and PD-L1 of a certain cell; comprises a PD-1 extracellular domain or a PD-1 transmembrane domain, or a combination thereof; comprises an intracellular domain comprising a substitution or deletion as compared to a wild-type PD-1 intracellular domain; or comprises a soluble receptor comprising a PD-1 extracellular domain that binds PD-L1 of a certain cell.

21. The modified cell of claim 30, wherein the modified cell is engineered to express and secrete a therapeutic agent, and optionally wherein the therapeutic agent is a cytokine, a small protein, or an agent regulated by Hif1a, NFAT, FOXP3, and/or NFkB.

22. The modified cell of claim 30, wherein the modified cell is a T cell derived from a healthy donor or a subject having cancer,

23. A composition comprising the modified cells of claim 30, wherein the modified cells comprise T cells.

24. (canceled)

25. A method of eliciting and/or enhancing T cell response in a subject having a solid tumor or treating a solid tumor in a subject, the method comprising administering an effective amount of T cell comprising the modified cell of claim 30.

26. The method of claim 25, wherein the tumor is associated with colorectal cancer, breast cancer, ovarian cancer, pancreatic cancer, lung cancer, liver cancer, endometrial cancer, kidney cancer, bladder cancer, or prostate cancer.

27-28. (canceled)

29. A modified cell comprising the vector of claim 16, wherein the modified cell comprises a T cell.

30. A modified cell comprising the CAR of claim 17, wherein the modified cell comprises a T cell.
